(19) United States
(12) Patent Application Publication
(10) Pub. No.: US 2025/0290123 A1
(43) Pub. Date: Sep. 18, 2025

(54) MULTIDOMAIN LIGASE ENZYMES WITH RNA-DNA HYBRID DUPLEX BINDING DOMAINS

(71) Applicant: 10x Genomics, Inc., Pleasanton, CA (US)

(72) Inventors: Ruijie Zhang, Milpitas, CA (US); Katherine Nakama, Dublin, CA (US); Mckenzi Toh, Pinole, CA (US); Aarushi Grover, Fremont, CA (US); Jessica Östlin, Stocksund (SE); Paulius Mielinis, Stockholm (SE)

(73) Assignee: 10x Genomics, Inc., Pleasanton, CA (US)

(21) Appl. No.: 19/076,775

(22) Filed: Mar. 11, 2025

Related U.S. Application Data
(60) Provisional application No. 63/564,213, filed on Mar. 12, 2024.

(51) Int. Cl. C12Q 1/6855; C12N 9/00; C12N 9/22; C12Q 1/6841

(52) U.S. Cl. CPC C12Q 1/6855; C12N 9/22; C12N 9/93; C12Q 1/6841; C12Y 605/01001

(57) ABSTRACT

The present disclosure provides chimeric ligase polypeptides comprising a ligase polypeptide domain operably linked to a catalytically inactive ribonuclease H (RNAseH) polypeptide domain; compositions, isolated nucleic acid, and vectors comprising the chimeric ligase polypeptides; and multiplexed methods for detecting nuclei acids in a biological sample using hybridization-based in situ sequencing assays.

### CROSS-REFERENCE TO RELATED APPLICATIONS

This application claims the benefit of priority to U.S. Provisional Application No. 63/564,213, filed Mar. 12, 2024, the content of which is hereby incorporated by reference in its entirety.

### FIELD OF DISCLOSURE

The present disclosure relates generally to the field of nucleic acid detection and amplification for profiling biological targets in biological samples. More specifically, the present disclosure relates to chimeric ligase polypeptides for the detection and amplification of nuclei acids based on target-specific ligation of oligonucleotides.

### BACKGROUND

Tools for profiling biological targets in a sample, such as genomics, transcriptomics, or proteomics profiling of cells, are essential for many purposes. For example, these tools can be used for identifying nucleic acids in a biological sample, understanding the molecular bases of cell identity, and/or for developing treatment for diseases. Many of these tools rely on ligation-based methods. These methods are essential for identifying sequences in a target nucleic acid and/or distinguishing between sequences of interest in a target nucleic acid.

The ligation-based methods take advantage of the unique properties of ligases, which are enzymes that catalyze the joining of two nucleic acid molecules by forming a new chemical bond. To perform this task, ligases must accurately discriminate between highly homologous nucleotide sequences, such as nucleotide sequences that differ in some instances only at the terminal nucleotide position. Ligation-based methods include, for example DNA-templated DNA ligation and RNA-templated DNA ligation. In the DNA-templated DNA ligation methods, the target nucleic acid and the oligonucleotide probe to be joined togethers are both deoxyribonucleotide (DNA) molecules (e.g., polymers); and the ligase is a DNA ligase. For the RNA-templated DNA ligation, the target nucleic acid is a ribonucleic acid (RNA) molecule, the oligonucleotide probe is a DNA molecule; and the ligase is either an RNA ligase or a DNA ligase.

Recently, efficient ligation of DNA probes to an RNA template has become the cornerstone of many applications in the field of nucleic acid analysis and engineering. However, current RNA-templated DNA ligation reactions suffer from lack of efficiency and specificity for multiple reasons, including but not limited to the properties of the ligase enzymes used. This is because ligases that can optimally hybridize DNA to an RNA (e.g., DN/RNA ligases) have yet to be found in nature. As such, DNA ligases that are able to weakly ligate DNA probes to an RNA target nucleic acid are used in RNA-templated DNA ligation reactions.

Accordingly, there is a need for efficient and specific DNA/RNA ligases for the detection and amplification of ribonucleic acids in biological samples using RNA-templated DNA ligation. The present disclosures addresses this need.

### SUMMARY OF THE DISCLOSURE

One aspect of the present disclosure provides a chimeric ligase polypeptide comprising a ligase polypeptide domain; and a catalytically inactive ribonuclease H (RNAseH) polypeptide domain.

In some embodiments, the catalytically inactive RNAseH polypeptide domain is located at the N-terminus of the ligase polypeptide domain. In some embodiments, the catalytically inactive RNAseH polypeptide domain is located at the C-terminus of the ligase polypeptide domain.

In some embodiments of the chimeric ligase polypeptide described herein, the ligase polypeptide domain comprises a T3 DNA ligase, a T4 DNA ligase, a T7 DNA ligase, a HiFi Taq DNA ligase, a 9° N™ DNA Ligase, an E. coli DNA ligase, a T4 RNA ligase, a Chlorella virus DNA ligase, a Paramecium bursaria Chlorella virus ligase, a Syngen Nebraska virus 5 ligase, or an Acanthocyslis turfacea chlorella virus ligase. In some embodiments, the ligase polypeptide domain comprises an Acanthocyslis turfacea chlorella virus ligase selected from the group consisting of ATCVMO0605SPH_239L, ATCV1_Z187L, ATCVWI0606_243L, ATCVNEJV2_239L, ATCVMN08101_251L, ATCVTN60342_234L, ATCVGM07011_240L, and ATCV-1 (LigAT). In some embodiments, the ligase polypeptide domain comprises a Paramecium bursaria Chlorella virus ligase selected from the group consisting of PBCV-1, KS1B, AN69C, CvsA1, NE-JV-4, CviKI, MA1D, NY2B, NYs1, PBCV-AR158, IL-5-2s1, MAlE, PBCV-IL3A, PBCV-NY2A, and AR158_C658R. In some embodiments, the ligase polypeptide domain comprises ATCV-1. In some embodiments, the ligase polypeptide domain comprises a T4 ligase (e.g., T4 DNA ligase or T4 RNA ligase).

In some embodiments of the chimeric ligase polypeptide described herein, the catalytically inactive RNAseH polypeptide domain is selected from the group consisting of Pyrococcus furiosus RNAseH, Pyrococcus horikoshi RNAseH, Pyrococcus woosii RNAseH, Bacillus sterothermophilus RNAse, Bacillus caldophilus RNAse, Thermococcus litoralis RNAseH II, Thermotoga neapolitana RNAse, Sulfolobus acidocaldarius RNAse, Thermoplasma acidophilum RNAseH, Thermus thermophilus RNAseH, Thermus thermophilus RNAseH I, Thermus thermophilus RNAseH II, Thermus thermophilus RNAseH III, Sulfobolus tokodaii RNAseH, Escherichia coli RNAseH, and a viral reverse transcriptase RNAse domain. In some embodiments, the catalytically inactive RNAseH polypeptide domain comprises a catalytically inactive E. coli RNAseH polypeptide.

In some embodiments, the catalytically inactive RNAseH polypeptide domain comprises a catalytically inactive Sulfobolus tokodaii RNAseH polypeptide. In some embodiments, the catalytically inactive RNAseH polypeptide domain comprises a catalytically inactive viral reverse transcriptase RNase domain. In some embodiments, the viral reverse transcriptase RNAse domain is a Moloney Murine Leukemia Virus (MMLV) Reverse Transcriptase RNAse domain.

In some embodiments of the chimeric ligase polypeptide described herein, the catalytically inactive RNAseH polypeptide domain comprises an amino acid sequence having at least about 90%, at least about 91%, at least about 92%, at least about 93%, at least about 94%, at least about 95%, at least about 96%, at least about 97%, at least about 98%, or at least about 99% sequence identity to any one of SEQ ID NO: 13, 31, or 33. In some embodiments, the catalytically inactive RNAseH polypeptide domain comprises an amino acid sequence set forth in SEQ ID NO: 13, 31, or 33.

In some embodiments, the catalytically inactive RNAseH polypeptide domain comprises a full-length E. coli RNAseH sequence or SEQ ID NO: 31. In some embodiments, the catalytically inactive RNAseH polypeptide domain comprises a full-length S. tokodaii RNAseH sequence, or SEQ ID NO: 33. In some embodiments, the catalytically inactive RNAseH polypeptide domain comprises the RNAseH domain of MMLV RT or SEQ ID NO: 13.

In some embodiments, the catalytically inactive RNAseH polypeptide domain comprises a mutation in any of one of SEQ ID NO: 13, 31, or 33, optionally, the mutation is selected from a substitution, an insertion, a deletion, or any combination thereof.

In some embodiments, the ligase polypeptide domain comprises an amino acid sequence having at least 90%, at least 91%, at least 92%, at least 93%, at least 94%, at least 95%, at least 96%, at least 97%, at least 98%, or at least 99% identity to SEQ ID NO: 29, 36, 38, or 39. In some embodiments, the ligase polypeptide domain comprises an amino acid sequence having at least 90% identical to SEQ ID NO: 29. In some embodiments, the ligase polypeptide domain comprises an amino acid sequence of SEQ ID NO: 29, 36, 38, or 39.

In some embodiments, the ligase polypeptide domain and the catalytically inactive RNAseH polypeptide domain are operably linked by a linker. In some embodiments, the linker is a peptide linker comprising a consensus sequence of G(n)S(m)G(o)C(p) and (a) n=0, 1, 2, 3, 4, 5, 6, 7, 8, 9, 10 11, 12, 13, 14, 15, 16, 17, 18, 19 or 20; (b) m=0, 1, 2, 3, 4, 5, 6, 7, 8, 9, 10 11, 12, 13, 14, 15, 16, 17, 18, 19 or 20; (c)p=0, 1, 2, 3, 4, 5, 6, 7, 8, 9, 1011, 12, 13, 14, 15, 16, 17, 18, 19 or 20; and (d) n, m, and p are selected independently. In some embodiments, the linker comprises an amino acid sequence having at least about 90%, at least about 91%, at least about 92%, at least about 93%, at least about 94%, at least about 95%, at least about 96%, at least about 97%, at least about 98%, or at least about 99% sequence identity to any one of SEQ ID NO: 17, 18, 19, or 20.

One aspect of the present disclosure provides a chimeric ligase polypeptide comprising: (a) a ligase polypeptide domain comprising the amino acid sequence of SEQ ID NO: 29; and (b) a catalytically inactive RNAseH polypeptide domain comprising the amino acid sequence of SEQ ID NO: 13, 31, or 33.

Another aspect of the present disclosure provides a chimeric ligase polypeptide comprising: (a) a ligase polypeptide domain comprising the amino acid sequence of SEQ ID NO: 36 and (b) a catalytically inactive RNAseH polypeptide domain comprising the amino acid sequence of SEQ ID NO: 13, 31, or 33.

Another aspect of the present disclosure provides a chimeric ligase polypeptide comprising: (a) a ligase polypeptide domain comprising the amino acid sequence of SEQ ID NO: 38 and (b) a catalytically inactive RNAseH polypeptide domain comprising the amino acid sequence of SEQ ID NO: 13, 31, or 33.

Another aspect of the present disclosure provides a chimeric ligase polypeptide comprising: (a) a ligase polypeptide domain comprising the amino acid sequence of SEQ ID NO: 39 and (b) a catalytically inactive RNAseH polypeptide domain comprising the amino acid sequence of SEQ ID NO: 13, 31, or 33.

In some embodiments of the chimeric ligase polypeptide described herein, the chimeric ligase polypeptide comprises an amino acid sequence that is at least 90%, at least 92%, at least 95%, at least 97%, at least 98%, or at least 99% identical to SEQ ID NO: 2, 4, 6, 8, 10, and 12. In some embodiments, the chimeric ligase polypeptide comprises the amino acid sequence of SEQ ID NO: 2, 4, 6, 8, 10, and 12. In some embodiments, the chimeric ligase polypeptide comprises the amino acid sequence of SEQ ID NO: 2.

In some embodiments of the chimeric ligase polypeptide described herein, the chimeric ligase polypeptide further comprises a tag protein selected from the group consisting of an affinity tag, a fluorescent tag, an expression, and solubility enhancement tag.

In some embodiments, the tag is selected from the group consisting of hexahistidine tag (his-tag), small ubiquitin-like modifier tag (SUMO), a VariFlex C-Terminal solubility enhancement tag, a short peptide C-terminal tag, Thioredoxin (Trx) tag, a VariFlex C-Terminal solubility enhancement tag, Solubility-enhancer peptide sequences (SET) tag, IgG domain B1 of Protein G (GB1) tag, IgG repeat domain ZZ of Protein A (ZZ) tag, Solubility enhancing Ubiquitous (SNUT) Tag, Seventeen kilodalton protein (Skp) tag, Phage T7 protein kinase (T7PK) tag, E. coli secreted protein A (EspA) tag, Monomeric bacteriophage T7 0.3 protein (Orc protein; Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen (Fh8) tag, Glutathione-S-transferase (GST) tag, maltose-binding protein (MBP) tag, Flag tag peptide (FLAG), streptavidin binding peptide (Strep-II; strep) tag, calmodulin-binding protein (CBP) tag, mutated dehalogenase (Halo) tag, staphylococcal Protein A (Protein A), intein mediated purification with the chitin-binding domain (IMPACT (CBD)), cellulose-binding module (CBM), dockerin domain of Clostridium josui (Dock) tag, and fungal avidin-like protein (Tamavidin).

In some embodiments, the tag is located at the N-terminus or C-terminus of the ligase polypeptide sequence. In some embodiments, the chimeric ligase polypeptide comprises an hexahistidine tag (his-tag). In some embodiments, the hexahistidine tag comprises an amino acid sequence of SEQ ID NO: 21; or an amino acid sequence having at least 90% sequence identity to the amino acid sequence of SEQ ID NO: 21.

In some embodiments of the chimeric ligase polypeptide described herein, the chimeric ligase polypeptide exhibits increased RNA-templated DNA ligation, increased ligation fidelity, enhanced ligase activity, improved thermostability, enhanced thermoreactivity, decreased ligation junction bias, or any combination thereof when compared to a control ligase not fused to an RNAseH domain.

In some embodiments, the increase in thermoreactivity, or thermostability is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to a wild-type ligase or a control ligase not fused to an RNAseH domain.

In some embodiments, the chimeric ligase polypeptide is active at a temperature of at least 55° C., at least 60° C., or at least 65° C.

In some embodiments, the chimeric ligase polypeptide produces the same ligation product yield at a reaction temperature of at least 55° C. as a ligation product yield produced as at a reaction temperature of 37° C.

In some embodiments, the chimeric ligase polypeptide produces at least about 2-fold to at least about 15-fold more ligation product at 37° C., 43° C., 48° C., 52° C., or 55° C. when compared to a wild-type ligase or a control ligase not fused to an RNAseH domain.

In some embodiments, the catalytically inactive RNAseH polypeptide domain enhances the binding of the chimeric ligase polypeptide to a RNA template, DNA probes, or RNA-templated DNA probes (e.g., RNA:DNA hybrid).

One aspect of the present disclosure provides an isolated nucleic acid molecule encoding any one of the chimeric ligase polypeptides described herein.

In some embodiments, the isolated nucleic acid molecule comprises a nucleotide sequence that is at least 70%, at least 75%, at least 80%, at least 90% identical to SEQ ID NO: 28, 30, 32, 34, 35, or 37.

In some embodiments, the isolated nucleic acid molecule comprises a nucleotide sequence that is at least 70%, at least 75%, at least 80%, at least 90% identical to SEQ ID NO: 1, 3, 5, 7, 9, or 11. In some embodiments, the isolated nucleic acid molecule comprises a sequence selected from SEQ ID NO: 1, 3, 5, 7, 9, 11, 28, 30, 32, 34, 35, or 37.

One aspect of the present disclosure provides an expression vector comprising any one of the isolated nucleic acid molecules described herein.

Another aspect of the present disclosure provides a host cell transfected with any one of the expression vectors described herein or any one of the isolated nucleic acid molecules described herein.

Another aspect of the present disclosure provides a composition comprising any one of the chimeric ligase polypeptides described herein.

Another aspect of the present disclosure provides a composition comprising any one of the isolated nucleic acid molecules described herein.

Another aspect of the present disclosure provides a composition comprising any expression vector described herein.

Another aspect of the present disclosure provides a composition comprising any host cell described herein. In some embodiments of the composition described herein, the composition further comprises a buffer.

Another aspect of the present disclosure provides a method for ligating double-stranded DNA or single-stranded DNA, the method comprising contacting under suitable conditions a sample comprising a nucleic acid template and DNA probes with a composition comprising a chimeric ligase polypeptide described herein.

In some embodiments, the composition further comprises a ligation buffer. In some embodiments, the ligation buffer has a pH in the range of about 8 to about 8.5. In some embodiments, the ligation buffer has a pH of at least about 8.0, 8.1, 8.2, 8.3, 8.4, or 8.5. In some embodiments, a ligation reaction as described herein is performed at a pH of about 8 or about 8.5.

In some embodiments, the sample comprises a cell, a cell bead, a permeabilized cell, a nucleus, a tissue, or a cellular preparation. In some embodiments, the nucleus is optionally permeabilized. In some embodiments, the sample comprises cells in suspension, fresh cells, fixed cells, or cells and tissues immobilized on various solid surfaces.

In some embodiments of the methods described herein, the nucleic acid template is an RNA, a DNA, or a nucleic acid comprising an unnatural nucleotide. In some embodiments, the nucleic acid template is an RNA template.

Another aspect of the present disclosure provides a method for detecting RNA sequences in a biological sample, the method comprising: contacting the biological sample with at least one DNA probe, where the at least one DNA probe comprise a 3′ DNA hybridization region and a 5′ DNA hybridization region that anneal to a first and a second complementary target sequences in the target RNA sequence in the biological sample; ligating the 3′ DNA hybridization region and the 5′ DNA hybridization region using the chimeric ligase polypeptide as provided herein to generate a ligation product; and detecting the ligation product, or an extension product or amplicon thereof, thereby detecting the RNA sequence.

In some embodiments, the DNA probe is a circularizable DNA probe. In some embodiments, the at least one DNA probe comprises a first DNA probe comprising a 3′ hybridization region, and a second DNA probe comprising a 5′ hybridization region. In some embodiments, the first and second complementary target sequences are adjacent in the target RNA sequence. In some embodiments, the first and second complementary target sequences are separated by one or more intervening nucleotides. In some embodiments, the first and second complementary target sequences are separated by a gap of about 1 to 40 nucleotides; about 1 to 60 nucleotides; or about 3 to 40 nucleotides.

In some embodiments, the gap is about 1, about 2, about 3, about 4, about 5, about 6, about 7, about 8, about 9, about 10, about 15, about 20 or more nucleotides. In some embodiments, the 3′ DNA hybridization region and the 5′ DNA hybridization region are ligated without gap filling.

In some embodiments of the methods described herein, the first and second complementary target sequences are separated by a gap and the method further comprises filling the gap prior to ligating the 3′ DNA hybridization region and the 5′ DNA hybridization region. In some embodiments, the gap is filled by extension of the 3′ DNA hybridization region of the DNA probe. In some embodiments, the gap is filled by one or more oligonucleotides which are complementary to the DNA probe, or the target RNA.

In some embodiments of the methods described herein, the biological sample is selected from the group consisting of: cells, nuclei, or cell beads, and the method further comprises partitioning single cells, single nuclei, or single cell beads into discrete partitions after the contacting and prior to the ligating. In some embodiments of the methods described herein, each of the discrete partitions comprises a barcode nucleic acid molecule comprising a partition-specific barcode. In that embodiment, the method further comprises, after the ligating: hybridizing the ligated product to the barcode nucleic acid molecules and extending the ligation product to generate extended ligation products comprising the partition-specific barcodes. In some embodiments, the ligated product comprises a sequence that hybridizes to the barcode nucleic acid molecules.

In some embodiments of the methods described herein, the biological sample comprises cells or tissue immobilized on a surface, and the method further comprises, after the ligating: contacting the biological sample with barcode nucleic acid molecules comprising spatial barcodes, hybridizing the ligated product to the barcode nucleic acid molecules, and extending the ligation product to generate extended ligation products comprising the spatial barcodes. In that embodiment, the ligated product comprises a sequence that hybridizes to the barcode nucleic acid molecules.

In some embodiments of the methods described herein, the method further comprises isolating the extended ligation product, and sequencing the isolated, extended ligation product, or amplicons thereof.

In some embodiments of the methods described herein, the sample comprises cells or tissue immobilized on a surface, and the method further comprises amplifying the ligation product, and the detecting comprises in situ detecting the amplified ligation product.

In some embodiments of the methods described herein, the at least one DNA probe is a circularizable probe, and the amplifying comprises rolling circle amplification. In some embodiments of the methods described herein, the at least one DNA probe comprises a barcode, optionally the barcode comprises a UMI. In some embodiments of the methods described herein, the in situ detecting comprises fluorescent probe hybridization and imaging.

Another aspect of the present disclosure provides a kit comprising: a chimeric ligase polypeptide described herein; or an isolated nucleic acid described herein; or an expression vector described herein; or a host cell described herein; or a composition described herein; and instructions.

### BRIEF DESCRIPTION OF THE DRAWINGS

FIG. 1 shows an AlphaFold model of a three-dimensional (3D) schematic representation of a chimeric ligase enzyme described herein. The chimeric ligase enzyme comprises a full-length ligase enzyme (e.g., LigAT) operably linked via a linker to a nucleic acid binding domain that can bind to RNA:DNA hybrid duplex (e.g., RNAseH from Escherichia coli (E. coli)). In the AlphaFold model, residues near C-terminus of LigAT have low pLDDT and positioning may have low accuracy.

FIG. 2 shows an image of an SDS-Page gel demonstrating the purity of a chimeric ligase enzyme (LigAT_c-RNAseH-E-coli) including LigAT ligase enzyme operably linked to a catalytically inactive RNAseH (c-RNAseH) from Escherichia coli (E. coli) using purification methods disclosed herein.

FIGS. 3A-C show electropherograms illustrating the results of a capillary electrophoresis (CE) RNAseH assay using exemplary chimeric ligase enzymes described herein (e.g., LigAT_c-RNAseH). FIGS. 3B-C show that RNAseH fused ligase enzymes comprising a mutation in the RNAseH domain were catalytically inactive for RNaseH activity (FIG. 3C) when compared to a catalytically active E. coli RNAseH (FIG. 3B). FIG. 3A shows a schematic of the CE RNAseH assay.

FIGS. 4A-C show electropherograms illustrating the results of a CE padlock ligation assay using an exemplary chimeric ligase enzyme described herein (e.g., LigAT_c-RNAseH), and showing that the chimeric ligase enzymes described herein were active for RNA templated DNA ligation. FIG. 4A shows a schematic assay of a CE padlock ligation assay. FIG. 4C shows that LigAT_C-RNAseH-ecoli was as active as a control ligase (LigAT-HU; FIG. 4B) in a control buffer at 37° C. The circular product is the ligation product generated by the chimeric ligase enzyme. app-DNA is a pre-adenylated single-stranded DNA (ssDNA) that was used as a substrate in the ligation reaction.

FIGS. 5A-E show ligation efficiency and percent yield of a ligation reaction intermediate (“app-DMA”), for a control ligase using a control buffer (LigAT fused to a DNA binding domain, HU; FIG. 5A), a ligase fused to E. coli RNAse H (LigAT_C-RNAseH-Ecoli), using the control buffer (FIG. 5B) or buffer 1B (FIG. 5C), and a ligase fused to Sto 7 RNAse H (LigAT_C-RNAseH-Sto7) using the control buffer (FIG. 5D) or buffer 2B (FIG. 5E).

FIGS. 6A-C show exemplary electropherograms illustrating the results of the CE padlock ligation assay of FIGS. 5A-E confirming that the chimeric ligase enzymes described herein showed improved thermostability. FIG. 6A shows an electropherogram of a control ligation reaction (LigAT-HU) in a control Buffer. FIG. 6B shows an electropherogram of a ligation reaction comprising the chimeric ligase enzyme, LigAT_C-RNaseH_Ecoli, in Buffer 1B. FIG. 6C shows an electropherogram of a ligation reaction comprising the chimeric ligase enzyme, LigAT_C-RnaseH_Sto in Buffer 2B.

FIGS. 7A-D show fluorescent microscopy images of a whole tissue using integrated in situ spatial assay performed using a microscope and analysis of optical signals for RNA sequence determination. Specifically, the chimeric ligase enzymes (e.g., catalytically inactive RNAseH fused ligase enzymes) described herein (FIGS. 7C-D) were active in situ and effective with higher temperature ligation reactions when compared to a control ligase (FIGS. 7A-B).

FIGS. 8A-D show the high sensitivity and specificity of the chimeric ligase enzymes disclosed herein in in situ spatial assays using different conditions (buffers, and temperatures) on fresh frozen murine brain (FFmbrain) tissues. FIG. 8A shows a schematic illustrating the calculation and definition of gene tissue density with Q-score ≥20 (in object per μm³).

FIG. 8B shows the in situ spatial assay sensitivity analysis using 3D spatial data analysis. The sensitivity of the in situ assay was defined as gene tissue density with Q-score ≥20 in object (sample) per μm³ (3D analysis) vs conditions (buffers and temperatures) shown on the x-axis. The conditions tested were two different buffers, Buffer 1A and Buffer 3A, compared to a control Buffer and three different temperatures (37° C., 42° C., and 50° C.). All the percentages on the graph represent fold change from LigAT-HU at 37° C. using control conditions. The control conditions were 2 hours at 37° C.

FIG. 8C shows the sensitivity analysis using 2D spatial data analysis. In situ assays comprising a LigAT_C-RNAseH-Ecoli enzyme were 8% more sensitive than control conditions when used at 50° C. in Buffer 1A. In situ assays comprising a LigAT_C-RNAseH-sto enzyme were 12% more sensitive than control conditions when used at 37° C. in Buffer 1A. In situ assays comprising a LigAT_C-RNAseH-RT enzyme were 10% more sensitive than control conditions when used at 37° C. in Buffer 1A.

FIG. 8D shows the high specificity of the chimeric ligase enzymes disclosed herein in in situ spatial assay using an adjusted negative control probe rate. The adjusted negative control probe rate measured both erroneous decoding and off-target binding, relative to the total number of high-quality transcripts detected. High quality transcripts are considered to be those with Q20 or above.

FIGS. 9A-C show that ligation junction bias can be monitored in vitro using orthogonal CE assays. FIG. 9A shows schematics of 8 probes and a template RNA for hybridization and ligation using a capillary electrophoresis (CE) assay for monitoring ligation junction bias. The 8 probes are divided into: 4 upstream oligonucleotides, each comprising a different base (e.g., A, C, G, and T) at the 3′end; and 4 fluorophore labeled downstream oligonucleotides, each including a different phosphorylated base (pA, pC, pG, and pT) at the 5′end. Each upstream oligonucleotide can pair with each fluorophore-labeled downstream oligonucleotide for a total of 16 reaction pairs (A_pA, A_pC, A_pG, A_pT, C_pA, C_pC, C_pG, C_pT, G_pA, G_pC, G_pG, G_pT, T_pA, T_pC, T_pG, T_pT), with each pairing complementary to one of sixteen different RNA templates.

FIG. 9B shows an exemplary electropherogram from a CE assay demonstrating the separation of the substrates (probe pairs) and the 16 expected exemplary ligated products described in FIG. 9A. All 16 products were separable in the CE assay.

FIG. 9C shows a schematic illustrating the nomenclature used for describing the CE ligation assay results. Specifically, for ligation junction “AC,” the ligation substrate comprised an upstream oligonucleotide having an A at the 3′end; and a labeled downstream oligonucleotide comprising pC to form a reaction pair ApC from 5′→3′ which formed a Watson-Crick bond with a complementary TG (3′→5′) on the template RNA. The two bases at the ligation junction on the template RNA are “AC” (reverse complement of the 5′→3).

FIGS. 10A-B show results of a multiplexed ligation fidelity and bias CE profiling assay using a control ligase enzyme (LigAT-HU). FIG. 10A shows an exemplary Table of percent yield of ligation product from a CE ligase fidelity assay. FIG. 10B shows a list ranking the ligation efficiency for each of the substrate combinations for the control ligase enzyme. The ligation reaction was performed using a control buffer with control ligation conditions (e.g., 2 hours at 37° C.).

FIG. 11 shows results of multiplexed ligation fidelity and bias profiling assays using a chimeric ligase enzyme described herein (LigAT_C-RNaseH-Ecoli). FIG. 11 demonstrates that an exemplary chimeric ligase enzyme described herein showed decreased ligation junction bias and improved fidelity when compared to a control (see FIG. 10A).

FIG. 12 shows an alignment demonstrating the relationship between SEQ ID NO: 13, and the RNAseH domain of the wild-type Moloney Murine Leukemia Virus (MMLV) Reverse Transcriptase and the RNAseH domain of the MMLV RT variant.

FIGS. 13A-B show results of in situ spatial assays using an exemplary chimeric ligase enzyme described herein (e.g., LigAT_C-RNaseH-Ecoli enzyme), and illustrating that the chimeric ligase enzyme improved the poor ligation junction sensitivity observed with a ligation reaction comprising a control enzyme (e.g., LigAT-HU) while maintaining other junctions. The in situ spatial assay was performed using formalin-fixed and paraffin embedded (FFPE) human cancer samples (TA, tpRCA) and evaluated with 1600 genes.

FIG. 13A shows a table summarizing the UMI counts and percent yield for each nucleotide pair at the ligation junction and demonstrating that the control enzyme showed poor junction sensitivity at ligation junctions GG, AG, CC, GC, CG, and AC in the in situ assay.

FIG. 13B shows graphs illustrating the relative sensitivity of the exemplary chimeric ligase enzyme (e.g., LigAT_C-RNaseH-Ecoli enzyme) compared to the control enzyme (e.g., LigAT-HU) in in situ assays. In particular, the relative sensitivity of the exemplary chimeric ligase enzyme was significantly improved at the CC junction in all tumor tissues tested (brain, colon, kidney, liver, lung, and pancreas). The relative sensitivity of the exemplary chimeric ligase enzyme was also significantly improved at the AC, AG, CG, GC, and GG junctions (shown in boxes).

FIGS. 14A-H show Uromodulin (UMOD) gene density data with Q-score ≥20 from in situ assays to identify using an exemplary chimeric ligase enzyme described herein (e.g., LigAT_C-RNaseH-Ecoli enzyme) and 4 ligation junction probes (CA, GC, TC, and AT); and demonstrating that an exemplary chimeric ligase enzyme (FIGS. 14E-H) showed less ligation junction bias in an in-situ spatial assay from a FFPE human Kidney sample when compared to a control enzyme (FIGS. 14A-D). The sensitivity of the in situ assay was defined as gene tissue density with Q-score ≥20 in object (sample). The ligation yields were 20-33% (CA), 10-20% (GC), and ≥34% (TC and AT). The sensitivity of the UMOD probe was significantly improved at the ligation junctions ‘GC’ in the modified workflow when LigAT_C-RNaseH-Ecoli was used compared to when LigAT-HU was used. The padlock set targeted 1000-genes and contained negative control probes.

FIGS. 15A-H show Aquaporin 2 (AQP2) gene density data with Q-score ≥20 from in situ assays using an exemplary chimeric ligase enzyme described herein (e.g., LigAT_C-RNaseH-Ecoli enzyme) and 4 ligation junction probes (CC, CT, TC, and TA); and demonstrating that an exemplary chimeric ligase enzyme (FIGS. 15E-H) showed less ligation junction bias in an in-situ spatial assay from a FFPE human Kidney sample when compared to a control enzyme (FIGS. 15A-D). The ligation yields were 10-20% (CC), and ≥34% (CT, TC, and TA). the sensitivity of the AQP2 probe was significantly improved at the ligation junctions ‘CC’ in the modified workflow when LigAT_C-RNaseH-Ecoli was used compared to when LigAT-HU was used. The padlock set targeted 1000-genes and contained negative control probes.

### DETAILED DESCRIPTION

### I. Overview

The present disclosure provides ligase enzymes with improved RNA templated DNA ligation activity.

Current high-throughput genomic applications rely on ligation-based methods for the sensitive and accurate detection of nucleic acid transcripts in biological samples. While the detection of DNA has been relatively straight forward, detecting and identifying RNA sequences in biological samples remain challenging for numerous reasons. For example, the detection of RNA from biological samples generally requires the conversion of RNA to cDNA by reverse transcription. Recently, RNA-templated DNA ligation was developed as a way of directly detecting RNA target using DNA probes and known or engineered ligases. RNA-templated DNA ligation was expected to be a sensitive and accurate detection of RNA transcripts using a ligation-based methods and DNA probes.

In RNA-templated DNA ligation, the target nucleic acid is an RNA molecule, the oligonucleotide probes are DNA molecules; and the ligase is an RNA ligase or a DNA ligase. However, natural ligase enzymes with RNA-templated DNA ligation activity are rare or uncharacterized due to the lack of biological need for RNA-templated DNA ligation. Currently, some DNA or RNA ligases that are able to weakly ligate DNA probes to a RNA target nucleic acid are used in RNA-templated DNA ligation reactions. Some ligases that have been tested include Chlorella virus DNA ligase (PBCV-1 DNA ligase I), T4 DNA ligase, T4 RNA ligase 1 (T4Rnl1), T4 RNA ligase 2 (T4Rnl2), DraRNI ligase, Acanthocystic turfacea chlorella virus 1 (“ATCV-1,” “ATCV1,” or “ATCV”). However, these ligases suffer from utility impacting limitations, including low activity for this non-native function (e.g., T4 DNA ligase), poor thermostability and salt tolerance (e.g., chlorella virus DNA ligase, also called SplintR), and strong ligation junction bias. Thus, current RNA-templated DNA ligation reactions suffer from lack of efficiency, fidelity, and specificity.

A. DNA Ligases are Prematurely Released from DNA-RNA Hybrid Before the Completion of the Ligation Reaction.

The enzyme T4 DNA ligase belongs to the DNA ligase family of enzymes and catalyzes the formation of a covalent phosphodiester bond from a free 3′ hydroxyl group on one DNA molecule and a free 5′ phosphate group of a second and separate DNA molecule. T4 DNA ligase is also able to ligate nicked DNA hybridized to a RNA strand. However, this reaction is not as efficient as a DNA-templated DNA ligation. This inefficiency is mainly caused by the premature release of T4 ligase from the nicked DNA/RNA hybrid molecule prior to the completion of the ligation reaction.

T4 DNA ligase catalyzes DNA ligation in three major steps. In the first step, the ligase binds the 3′ hydroxyl ends of one DNA molecule (i.e., acceptor) with the 5′ phosphate end of another DNA molecule (i.e., donor). Following the reorganization of the DNA fragments within the active site of the ligase, a lysine residue within the activate site of the ligase is adenylated with the addition of AMP through ATP hydrolysis, and a pyrophosphate is released. In the second step, the ligase transfers the AMP to the 5′ phosphate end of the donor (the phosphate at the 5′ end of the nick). This transfer results in the formation of an intermediate product possessing a 5′ to 5′ pyrophosphate bond. In the last step, the 5′ phosphate end of the donor is linked to the 3′ hydroxyl end of the acceptor, which generates the formation of the phosphodiester bond. Specifically, the ligase catalyzes the formation of a phosphodiester bond by attacking the intermediate 5′ to 5′ pyrophosphate bond with the OH group of the 3′ end of the acceptor. The ligase and AMP are released from the DNA duplex following the formation of the covalent phosphodiester bond in the ligated product. Because the ligase does not bind to the 5′ to 5′ pyrophosphate bond intermediate, premature release of the ligase from the DNA duplex after the formation of the intermediate product terminates the ligation reaction.

In the RNA-templated DNA ligation, the T4 DNA ligase usually completes step 1 and step 2. However, the T4 DNA ligase is generally prematurely released from the DNA:RNA hybrid after the formation of the 5′ to 5′ pyrophosphate bond intermediate species. The release of T4 DNA ligase prevents the ligase from completing the final step of the reaction resulting in the accumulation of 5′ to 5′ pyrophosphate bond intermediate product. As such, T4 DNA ligase-mediated reaction terminates before the ligated product is generated (i.e., the end of the reaction). This failure is believed to be the source of the low ligation efficiency in RNA-templated DNA ligation. Thus, there is a need for novel ligases that can selectively and effectively function in RNA-templated DNA ligation reactions.

### B. A Novel Chimeric Ligase Enzyme Design

The present disclosure provides novel engineered ligase enzymes with multiple domains that allow the engineered ligase enzymes to bind strongly to the RNA-DNA hybrid duplex during a RNA templated DNA ligation reaction thereby preventing the premature release of the ligase from the RNA-DNA hybrid duplex and the subsequent premature termination of the ligation reaction. The novel multidomain ligase enzymes were developed using a novel design. In the design, a ligase enzyme (e.g., a DNA ligase) is fused to an heterologous protein or a domain of a protein that can specifically bind RNA-DNA hybrid duplexes. The present disclosure demonstrates that such chimeric ligase enzymes were active for RNA templated DNA ligation and exhibited improved ligation properties compared to the single domain ligase or a control ligase.

For the novel design disclosed herein, the ligase can be any DNA or RNA ligase, including but not limited to ligase that have been reported to have RNA templated DNA ligation activity. The RNA-DNA hybrid duplex binding protein or binding domain can be any protein reported to have binding affinity for RNA-DNA hybrid duplex. These proteins can include, but are not limited to, ribonuclease H (RNAseH) proteins and other regulatory proteins. RNAseH is a protein capable of selectively binding RNA-DNA hybrid duplex despite an excess of double stranded DNA. Some enzymes, which act on RNA-DNA hybrid duplexes (like reverse transcriptase), have an RNAseH domain. In the event the binding domain is an enzyme, the catalytic activity of the binding domain can be inactivated by mutation. The RNA-DNA hybrid duplex binding domain can be fused either to the N- or C-terminus of the ligase enzyme.

The multidomain ligase enzymes (also called chimeric ligase polypeptide) can further comprise a linker and/or one or more tags. The linker can be any length (0 to ≥20 amino acids) and can be comprised of numerous combinations of amino acids. The tags can be used for but not limited to purification and solubility. Exemplary tags can include, but not limited to an N-terminal histidine (N-His) tag, a C-terminal histidine (C-His) tag, or a C-His SET tag as described herein.

The approach disclosed herein generated multidomain ligase enzymes with RNA-templated DNA ligation activity and improved properties including increased thermostability & fidelity and decreased ligation junction bias. These properties have direct benefit to RNA-templated ligation dependent workflows, including increased probe design space and the potential to enable single nucleotide variation (SNV) workflows. The novel chimeric ligase polypeptides or enzymes described herein allow for fast, efficient, and low-sequence bias in RNA-templated DNA ligation during high-throughput genomic applications, such as for example in situ assays, and NGS-based spatial assays and single cell assays.

C. Novel Chimeric Ligase Enzymes with an Enhanced RNA-DNA Hybrid Duplex Binding Domain

Several multidomain ligase enzymes in which a ligase (e.g., DNA ligase) is fused with a RNA-DNA hybrid duplex binding protein were synthesized and characterized.

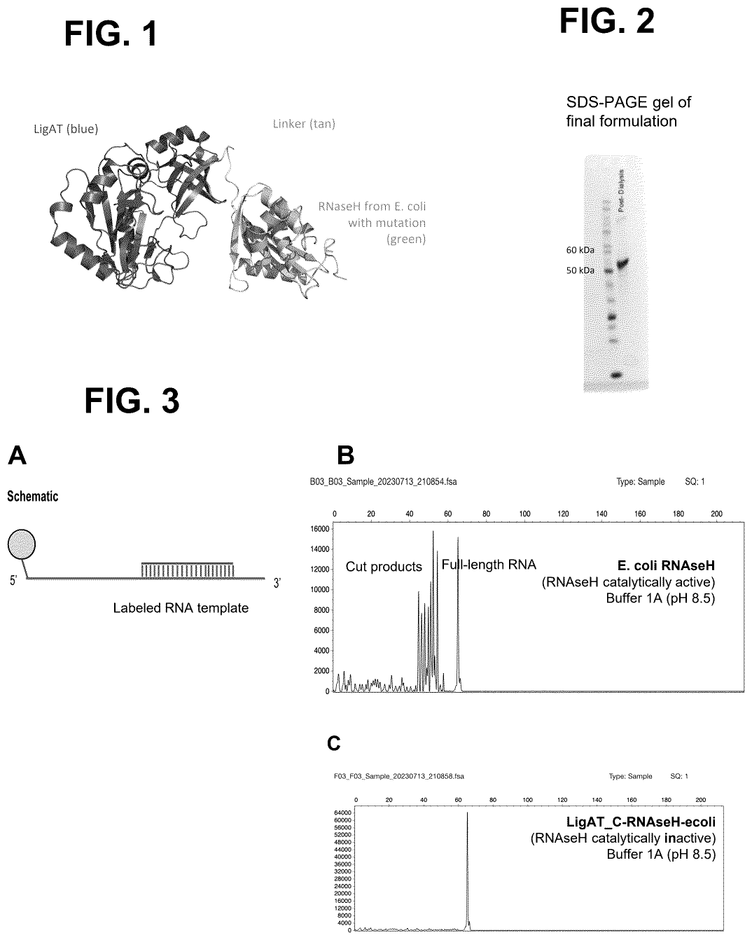

Exemplary chimeric ligases comprise a ligase polypeptide domain operably linked to a catalytically inactive RNAseH polypeptide domain. The RNAseH polypeptide domain is either a full-length RNAseH from any organism or a catalytically inactive RNAseH domain from any molecule containing an RNAseH domain (e.g., a reverse transcriptase). The linker chosen in these constructs, which comprises 6 amino acids, was designed to provide flexibility and solubility. However, other linkers may also be suitable or even offer improved performance. The exemplified ligases were selected from (Acanthocystic turfacea chlorella virus 1 (“ATCV-1,” “ATCV1,” “ATCV,” or “LigAT), T4 DNA ligase, or T4RNA ligase. The exemplified inactive RNAseH polypeptide domains were selected from E. coli RNAseH, Sulfobolus tokodaii RNAseH, or the RNAseH domain of an MMLV RT variant. A schematic structure of one novel chimeric polypeptide comprising LigAT ligase domain fused to E. coli RNAseH (SEQ ID NO: 1 and 2) is shown in FIG. 1.

### 1. Improved Efficiency

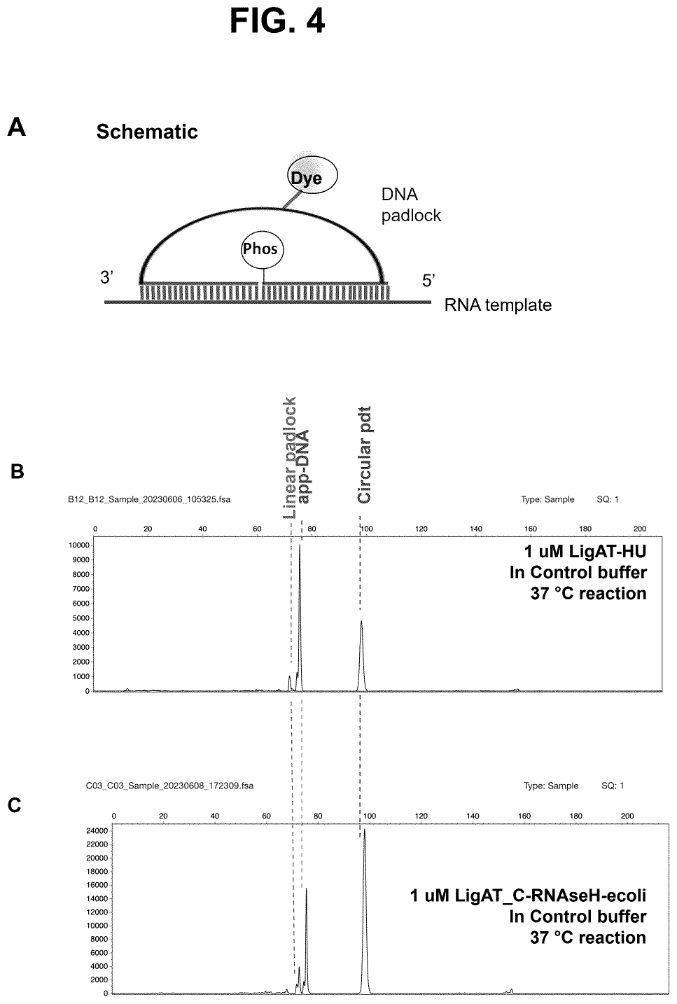

The characterization of the exemplary novel chimeric RNAseH-ligase enzymes disclosed herein showed that RNAseH domain was catalytically inactive (FIGS. 3A-C). The novel chimeric enzymes were very active ligase for RNA-templated DNA ligation (FIGS. 4-7). Moreover, the chimeric enzymes showed improved efficiencies when compared to wild-type LigAT or a recombinant ligase comprising a regular DNA binding domain (LigAT-HU). LigAT-HU, which is also a fusion of a ligase with a second protein was used as control. LigAT-HU is a fusion of LigAT ligase to histone-like HU protein, which is a double stranded DNA binding protein.

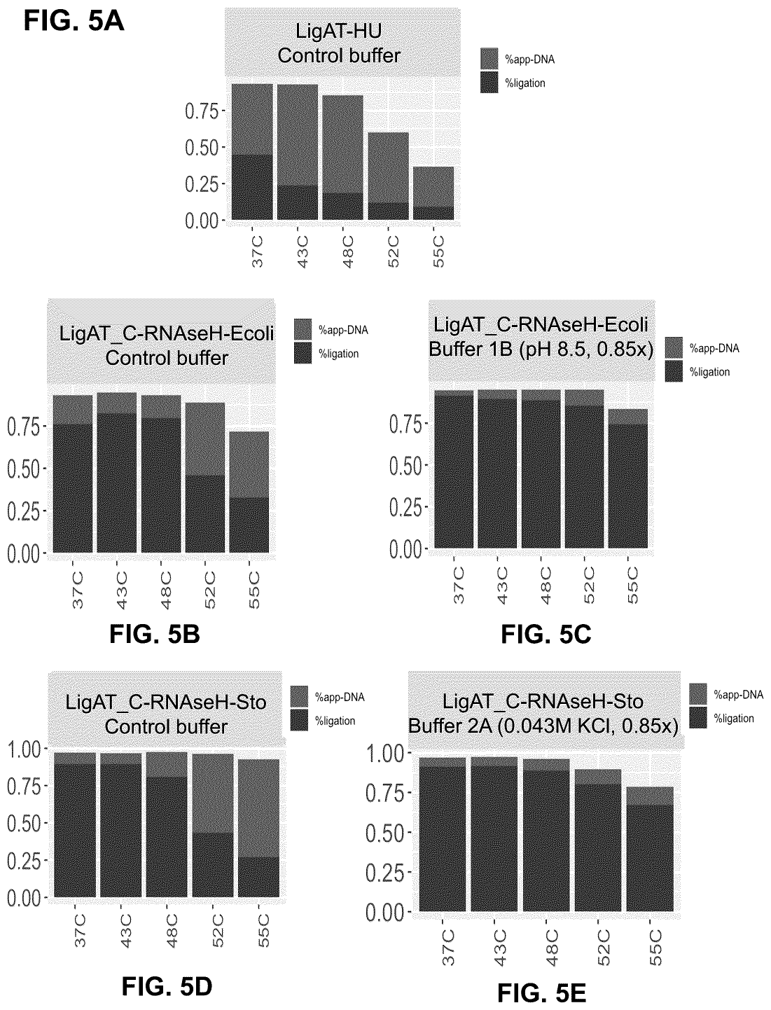

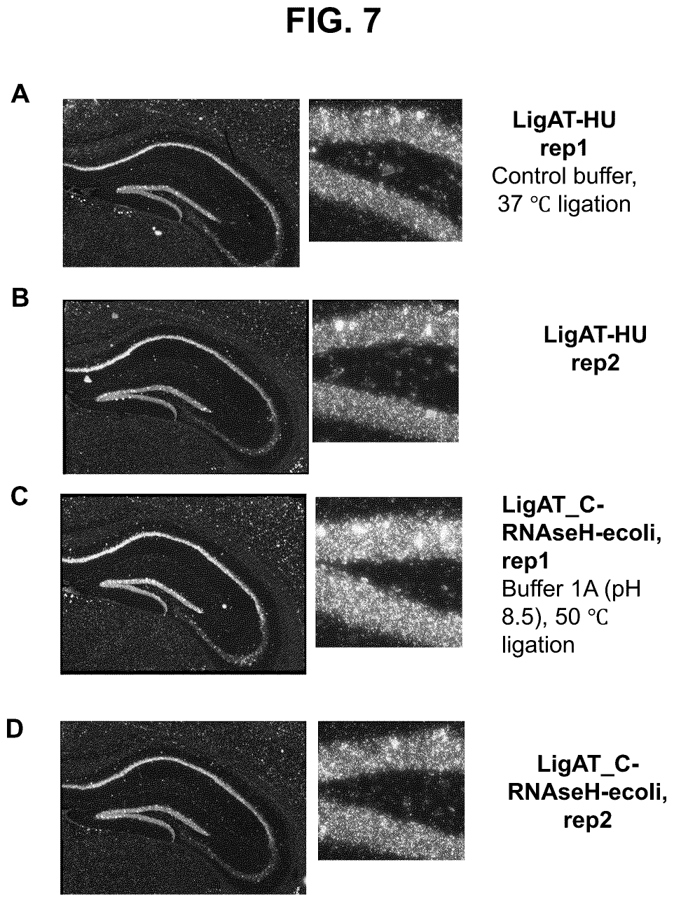

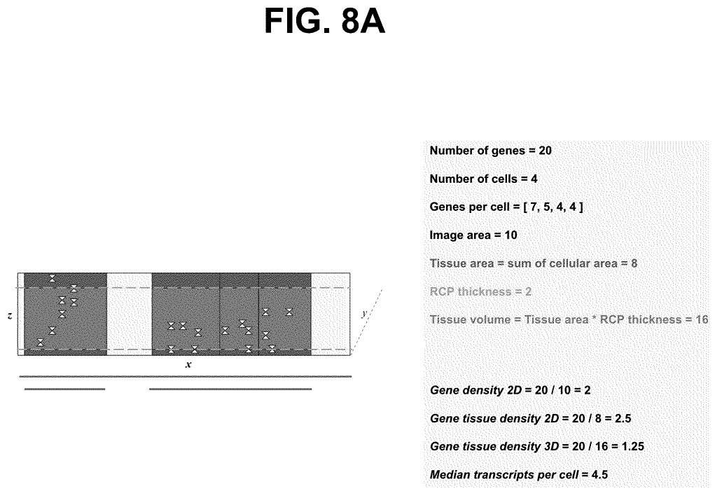

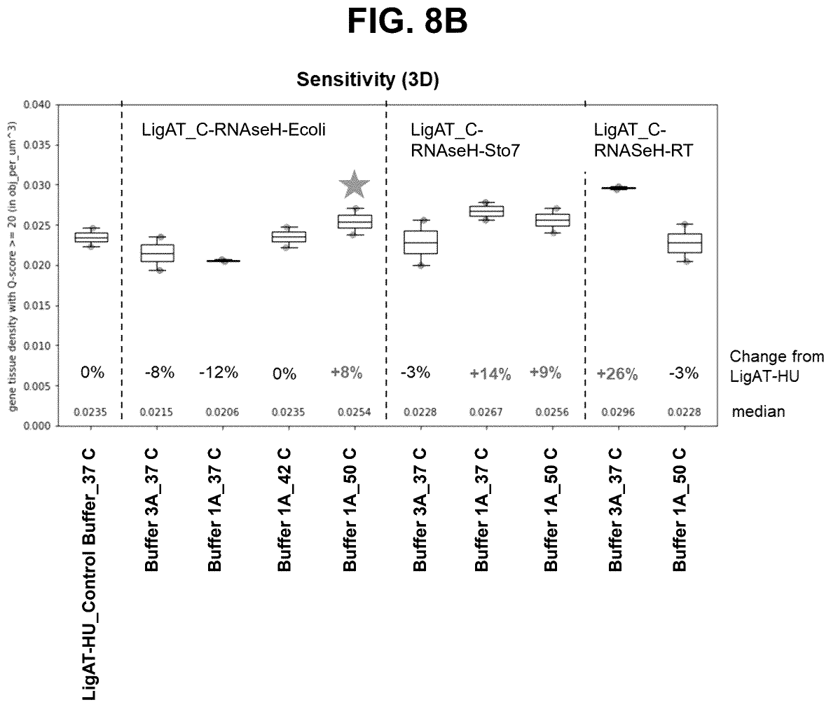

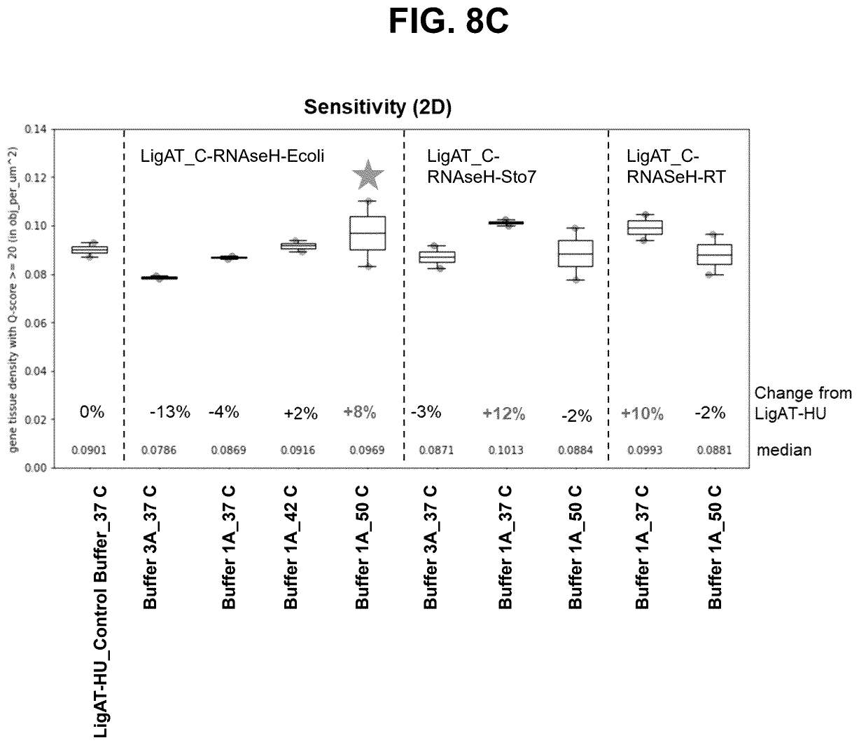

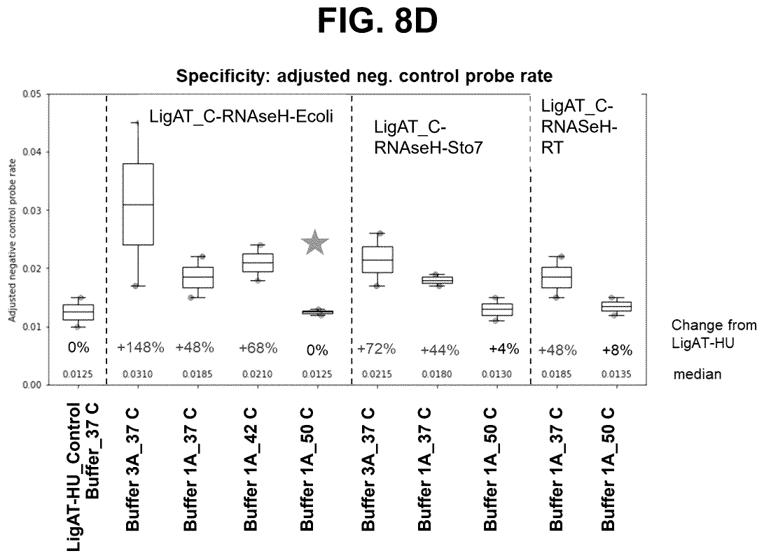

FIGS. 4C, 5B-E, 6B-C, and 7C-D show that RNA-templated DNA ligation reaction comprising the chimeric ligase disclosed herein showed reduced accumulation of the 5′ to 5′ pyrophosphate bond intermediate product (i.e., app-DNA) when compared to FIGS. 4B, 5A, 6A, and 7A-B (LigAT-HU; control). As such, the novel enzymes were not as prematurely released from the template compared to the control Ligase. In addition, the chimeric ligase enzymes disclosed herein showed potential for cross functional use between spatial assay and in situ assay (FIGS. 7A-D and FIG. 8A-B).

### 2. Improved Thermostability

The chimeric ligase described herein also showed improved thermostability when compared to the corresponding wild-type ligase (e.g., LigAT) or a recombinant ligase fused to DNA binding domain (e.g., a non-RNAseH or LigAT-HU) (FIGS. 5A-E). Indeed, the RNA-templated DNA ligation was efficient at all temperatures tested based on the percentage of ligation product obtained compared to the percentage of the 5′ to 5′ pyrophosphate bond intermediate product (i.e., app-DNA). In Buffer 1A, Buffer 1B and Buffer 2B, over 75% of the nucleic acids at the end of the assay was the final ligated product compared to less than 10% app-DNA substrate (i.e., the 5′ to 5′ pyrophosphate bond intermediate product) at 37° C., 43° C., 48° C., 52° C., and 55° C. (FIGS. 5C and E). In the control buffer, about 75% of the nucleic acids at the end of the assay was the final ligated product at 37° C., 43° C., or 48° C. At 52° C., and 55° C., the percentage of final ligated product in the reaction remained above 25-30%. In contrast, the control ligase (LigAT-HU), produced only about 25% ligated product at 37° C. in control buffer, and that percentage was reduced to less than 5% at 55° C. (FIG. 5A).

Furthermore, the chimeric ligase enzymes were thermostable when compared to wild-type enzyme. As such, the present disclosure provides novel RNA-templated DNA ligases for higher temperature ligation (>37° C.). Higher temperature ligation is beneficial and desired for ligase fidelity and ligase-dependent single nucleotide variation (SNV) detection.

### 3. Reduction of Ligation Junction Biases

DNA ligases canonically join a 3′-hydroxyl terminus of DNA molecule to a 5′-phosphoryl group terminus, for example, when both form complementary base-pairing with a template strand. Ligation junction bias relates to a bias or preference toward ligating certain nucleotides together, over ligating other nucleotide pairs together. Identification and remediation of nucleotide bias in ligation reaction is crucial because such bias can fail to quantitatively capture the original information stored in the DNA or RNA sample. For example, ligation bias can result in false negative results, when nucleic acid samples include ligatable nucleotide ends that are not ligated during a reaction due to a ligation bias against these nucleotide ends. In some embodiments, provided herein are chimeric ligase enzymes that reduce ligation bias, as compared to a control ligase enzyme (e.g., a same ligase that is not fused to an RNAse H domain, or a same ligase that is fused to a DNA binding domain).

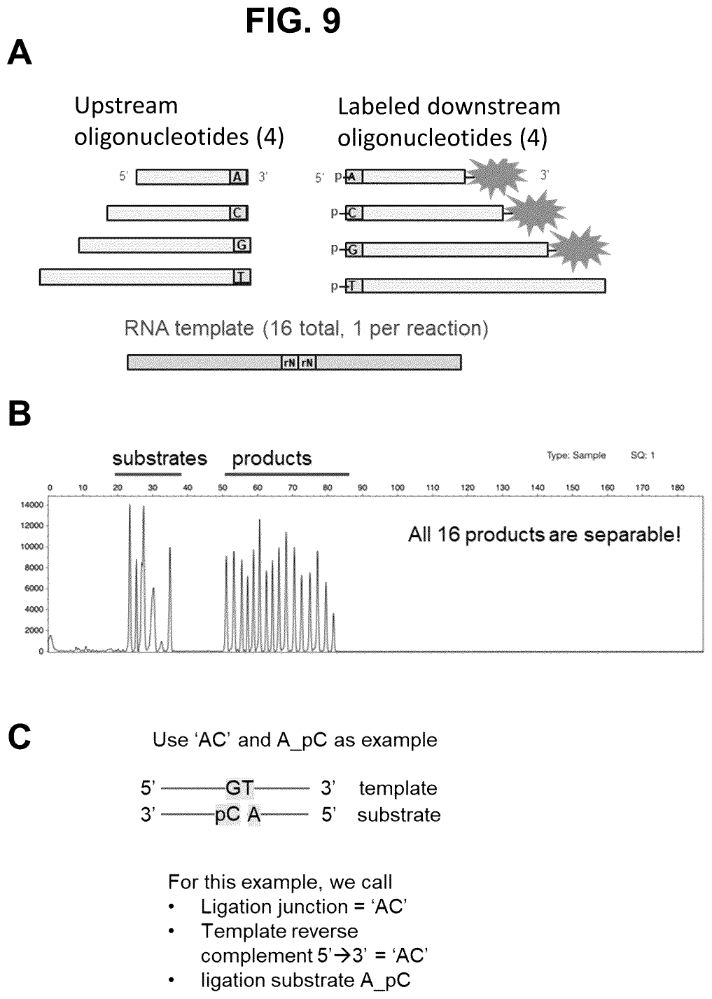

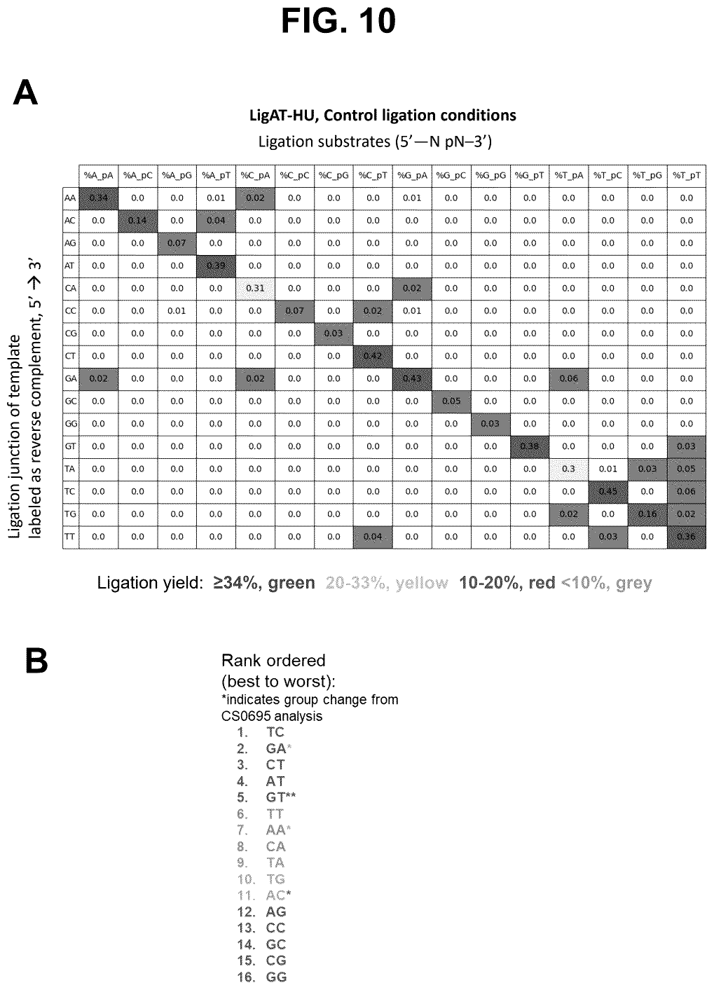

For example, an amplification product can be generated to detect an RNA transcript using a conventional padlock probe, by hybridizing free ends of the padlock probe to the RNA and ligating the free ends together using an efficient ligase. However, if a ligase has a ligation bias against a particular nucleotide end ligation junction, this can limit possible sequences targeted in the design of probe panels. Accordingly, a comprehensive evaluation of the junction bias of the chimeric ligase enzymes of the present disclosure was performed as shown in FIGS. 9-10. The chimeric ligase described herein showed decreased ligation junction bias when compared to LigAT-HU.

Therefore, the chimeric ligases described herein showed improved RNA templated ligation of nucleic acid probes. This led to decreased ligation junction bias during single cell profiling. In addition, it was observed that the addition of the RNAseH domain to the ligase enzyme, (i.e., the fusion domains) improved the chimeric ligase protein stability during a reaction, and particularly at higher temperatures.

### II. Chimeric Ligase Polypeptides

One aspect of the present disclosure provides a chimeric ligase polypeptide comprising, consisting essentially of, or consisting of a ligase polypeptide domain; and a catalytically inactive ribonuclease H (RNAseH) polypeptide domain. In one embodiment, the catalytically inactive RNAseH polypeptide domain is located at the N-terminus of the ligase polypeptide domain. In another embodiment, the catalytically inactive RNAseH polypeptide domain is located at the C-terminus of the ligase polypeptide domain. In some embodiments, the ligase polypeptide domain and the catalytically inactive RNAseH polypeptide domain are operably linked by a linker.

### A. Any Ligases can be Used to Engineer the Chimeric Ligase Polypeptide

A Ligation comprises the formation of a phosphodiester bond between the 3′ OH group at the 3′ of a probe and the 5′ phosphate group at the 5′ end of a probe in two adjacent bases hybridized to a target nucleic acid sequence and juxtaposed for ligation. Depending on the design of the probe, the 3′ OH group and/or 5′ phosphate group may be provided on a ribonucleotide (RNA) or a deoxyribonucleotide (DNA). Accordingly, a ligase enzyme may be selected, which is capable of catalyzing the formation of a phosphodiester bond in a target-specific manner (e.g., a DNA ligase or an RNA ligase).

A ligase refers to a family of enzymes that catalyzes the formation of a covalent phosphodiester bond between two distinct nucleic acid strands (i.e., a ligation reaction).

Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), or EC 6.5.1.3 (RNA ligases). Specific examples of ligases comprise bacterial ligases such as E. coli DNA ligase, Thermus thermophilus (T. thermophilus) DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof.

The ligase contemplated by the present disclosure can be any DNA or RNA ligase, including but not limited to ligase that have been reported to have RNA templated DNA ligation activity. In some embodiments of the chimeric ligase polypeptide described herein, the ligase may be a DNA ligase. A DNA ligase is a ligase enzyme characterized by its ability to catalyze the formation of a phosphodiester bond between two adjacent deoxyribonucleotides (DNA) hybridized to a target nucleic acid molecule. In some embodiments of the chimeric ligase polypeptide described herein, the ligase may be an RNA ligase. An RNA ligase is a ligase enzyme characterized by its ability to catalyze the formation of a phosphodiester bond between two adjacent ribonucleotides hybridized to a target nucleic acid molecule.

Two prokaryotic DNA ligases, (1) the ATP-dependent T4 DNA ligase (isolated from the T4 phage), and (2) the NAD+-dependent DNA ligase from E. coli, have become indispensable tools in molecular biology applications. Both enzymes catalyze the synthesis of a phosphodiester bond between the 3′-hydroxyl group of one polynucleotide, and the 5′-phosphoryl group, of a second polynucleotide, for instance at a nick between the two strands, which are both hybridized to a third DNA strand. The mechanism of the ligation reaction catalyzed by this family of enzymes typically requires three enzymatic steps.

DNA ligase catalyzes DNA ligation in three major steps. In the first step, the ligase binds the 3′ hydroxyl ends of one nucleotide (i.e., acceptor) with the 5′ phosphate end of another nucleotide (i.e., donor). Following the reorganization of the DNA fragments within the active site of the ligase, a lysine residue within the activate site of the ligase is adenylated with the addition of AMP through ATP hydrolysis, and the release of pyrophosphate. In the second step, the ligase transfers the AMP to the 5′ phosphate end of the donor (the phosphate at the 5′ end of the nick). This transfer results in the formation of an intermediate product possessing a 5′ to 5′ pyrophosphate bond. In the last step, the 5′ phosphate end of the donor is then linked to the 3′ hydroxyl end of the acceptor, which generates the formation of the phosphodiester bond. Specifically, the ligase catalyzes the formation of a phosphodiester bond by attacking the intermediate 5′ to 5′ pyrophosphate bond with the OH group of the 3′ end of the acceptor. The ligase and AMP are released from the RNA:DNA hybrid following the formation of the covalent phosphodiester bond in the ligated product. Because the ligase does not bind to the 5′ to 5′ pyrophosphate bond intermediate, premature release of the ligase from the DNA:RNA hybrid after the formation of the intermediate product terminates the ligation reaction.

DNA ligases are able to distinguish single nucleotide variations among DNA sequences and therefore may be exploited in such expression profiling analyses. DNA ligases are relatively inefficient in ligating two nucleic acid sequences possessing non-complementary termini. This property of the ligase enzyme has been exploited in numerous in vitro assays, such as e.g., the oligonucleotide ligation assay (OLA); the ligase chain reaction.

Any ligase can be used to engineer the chimeric ligase disclosed herein.

As shown in the Examples, both DNA ligase enzymes (T4 DNA ligase, LigAT, or PBCV-1DNA ligase) and RNA ligase enzymes (e.g., T4 RNA ligase2) have now been shown to catalyze the formation of phosphodiester bonds between an adjacent 3′ ribonucleotide and a 5′ deoxyribonucleotide, and between an adjacent 3′ ribonucleotide and a 5′ ribonucleotide, which are hybridized to a target nucleic acid molecule, in an efficient and target-dependent manner. Thus, both DNA and RNA ligases may be used in the chimeric ligase polypeptide described herein. Accordingly, reference to a “ligase polypeptide” includes both DNA ligase and RNA ligases which are capable of joining hybridized 3′ and 5′ nucleotides to form a ligation product comprising a ribonucleotide at or near to the ligation site as described herein.

In some embodiments of the chimeric ligase polypeptide described herein, the ligase polypeptide domain can comprise, consist essentially of, or consist of a T3 DNA ligase, a T4 DNA ligase, a T4 RNA ligase 1 (T4Rnl1), a T4 RNA ligase 2(T4Rnl2), a Dra RNA ligase, a T7 DNA ligase, a HiFi Taq DNA ligase, a 9° N™ DNA Ligase, an E. coli DNA ligase, a Chlorella virus DNA ligase, a Paramecium bursaria Chlorella virus ligase, a Syngen Nebraska virus 5 ligase, or an Acanthocyslis turfacea chlorella virus ligase.

In some embodiments, the ligase is from a Phycodnaviridae virus. In some embodiments, the ligase is from a viral family selected from the group consisting of Poxviridae, Iridoviridae, Ascoviridae, Asfarviridae, and Mimiviridae. In some embodiments, the ligase is from a Paramecium bursaria Chlorella selected from PBCV-1, KSIB, AN69C, CvsA1, NE-JV-4, CviKI, MAID, NY2B, NYs1, AR158 (PBCV-AR158), IL-5-2s1, MA1E, PBCV-IL3A), PBCV-NY2A), AR158_C658R.

In some embodiments, the ligase polypeptide domain can comprise a Acanthocyslis turfacea chlorella virus ligase selected from the group consisting of ATCVMO0605SPH_239L, ATCV1_Z187L, ATCVWI0606_243L, ATCVNEJV2_239L, ATCVMN08101_251L, ATCVTN60342_234L, ATCVGM07011_240L, and ATCV-1 (LigAT). The ligase polypeptide domain can comprise, consist essentially of, or consist of a Paramecium bursaria Chlorella virus ligase selected from the group consisting of PBCV-1, KS1B, AN69C, CvsA1, NE-JV-4, CviKI, MAID, NY2B, NYs1, PBCV-AR158, IL-5-2s1, MA1E, PBCV-IL3A, PBCV-NY2A, and AR158_C658R.

In some embodiments, the ligase polypeptide domain comprises ATCV-1. The ATCV-1 ligase is specific to Acanthocystis turfacea and does not infect or inhabit Paramecium bursaria. The ATCV-1 ligase has a predicted molecular weight of approximately 33.71 kDa. The ATCV-1 ligase can be expressed in soluble form in Escherichia coli and purified to homogeneity. Purified ATCV-1 ligase is able to efficiently ligate DNA probes hybridized to an RNA template. Moreover, ATCV-1 ligase's activity and performance are similar to those of the PBCV-1 enzyme after normalizing the concentrations of the two enzymes.

Any functional ligase can be used to engineer the chimeric ligase enzymes described herein for RNA templated DNA ligation. A ligase contemplated by the present disclosure can be any DNA or RNA ligase. The specific ligase examples provided should have reported RNA templated DNA ligation activity.

In some embodiments, the ligase polypeptide domain comprises an amino acid sequence having at least about 90%, at least about 91%, at least about 92%, at least about 93%, at least about 94%, at least about 95%, at least about 96%, at least about 97%, at least about 98%, or at least about 99% identity to SEQ ID NO: 29. In one embodiment, the ligase polypeptide domain comprises an amino acid sequence having at least 90% identical to SEQ ID NO: 29. In one embodiment, the ligase polypeptide domain comprises an amino acid sequence of SEQ ID NO: 29.

In some embodiments, the ligase is from Paramecium bursaria Chlorella virus 1 (PBCV1). PBCV1 is a DNA virus that infects green algae and encodes the smallest known eukaryotic ATP-dependent DNA ligase. Although PBCV1 ligase lacks the large accessory domains found in cellular DNA ligases, it can sustain mitotic growth and DNA repair in Saccharomyces cerevisiae (budding yeast) when it is the only ligase available.

In some embodiments, the ligase polypeptide domain comprises a PBCV-1 DNA ligase. PBCV-1 is also known commonly and referred to as SplintR® Ligase or Chlorella virus DNA Ligase. In some embodiments, the ligase polypeptide domain comprises an amino acid sequence having at least about 90%, at least about 91%, at least about 92%, at least about 93%, at least about 94%, at least about 95%, at least about 96%, at least about 97%, at least about 98%, or at least about 99% identity to SEQ ID NO: 39.

Based on sequence alignment analyses, the ATCV-1 ligase of the present disclosure shares only approximately 53 percent (52.86%) amino acid sequence identity with PBCV-1 ligase. Despite their lack of significant sequence homology, there is a high degree of structural similarity based on a comparison of the structural model of ATCV-1 ligase with the crystal structure of PBCV-1 ligase.

As described herein, direct detection of RNA target-DNA probe duplexes (without first converting RNA to cDNA by reverse transcription) is difficult given that a majority of DNA ligases fail to ligate nicked DNA hybridized to an RNA template. T4 DNA ligase has been shown to catalyze the ligation of DNA oligonucleotides hybridized to RNA templates. However, this function of T4 DNA ligase is substantially reduced in catalytic efficiency as compared to its catalyzation of DNA-templated reactions. Nonetheless, under low salt, particularly monovalent cations, and low ATP concentrations, high concentrations of T4 DNA ligase efficiently join DNA oligonucleotides hybridized in juxtaposition on RNA target strands.

In some embodiments, the ligase polypeptide domain comprises a T4 DNA ligase. T4 DNA ligase belongs to the DNA ligase family of enzymes (E.C. 6.5.1.1), which catalyzes the formation of a covalent phosphodiester bond from a free 3′ hydroxyl group on one DNA molecule and a free 5′ phosphate group of a second, separate DNA molecule. Like many ligases, T4 DNA ligase activity requires adenosine triphosphate (ATP) as a cofactor. Recombinant T4 DNA ligase, and various mutants thereof, is commercially available. T4 DNA ligase can also ligate double-stranded molecules, such as for example, DNA/DNA, DNA/RNA hybrids, and RNA/RNA.

In some embodiments, the ligase polypeptide domain comprises an amino acid sequence having at least about 90%, at least about 91%, at least about 92%, at least about 93%, at least about 94%, at least about 95%, at least about 96%, at least about 97%, at least about 98%, or at least about 99% identity to SEQ ID NO: 36.

In some embodiments, the ligase polypeptide domain comprises a T4 RNA ligase. In some embodiments, the ligase polypeptide domain comprises an amino acid sequence having at least about 90%, at least about 91%, at least about 92%, at least about 93%, at least about 94%, at least about 95%, at least about 96%, at least about 97%, at least about 98%, or at least about 99% identity to SEQ ID NO: 38.

### B. An RNAseH Domain as an RNA-DNA Hybrid Duplex Binding Domain

The present disclosure provides novel engineered ligase enzymes with one or more heterologous domains that can allow the ligase to bind strongly to the RNA-DNA hybrid duplex during a ligation reaction thereby preventing the premature release of the ligase from the RNA-DNA hybrid duplex and the subsequent premature termination of the ligation reaction. In the novel design described herein, an engineered ligase as described herein include a ligase enzyme (e.g., a DNA ligase) fused to an heterologous protein or a domain of an heterologous protein that can specifically bind RNA-DNA hybrid duplexes. The RNA-DNA hybrid duplex binding domain can be fused either to the N- or C-terminus of the ligase enzyme. In some embodiments, the protein or a domain of a protein that can specifically bind RNA-DNA hybrid duplexes is not endogenous to the ligase.

The RNA-DNA hybrid duplex binding domain can be any protein reported to have a binding affinity for a RNA-DNA hybrid duplex. These proteins can include, but are not limited to, RNAseH proteins and other regulatory proteins. RNAseH is a protein capable of selectively binding RNA-DNA hybrid duplexes, even when an excess of double stranded DNA is present.

The RNAseH or a polypeptide fragment comprising the RNA:DNA hybrid duplex binding domain described herein can be located at the N-terminus of the ligase polypeptide sequence.

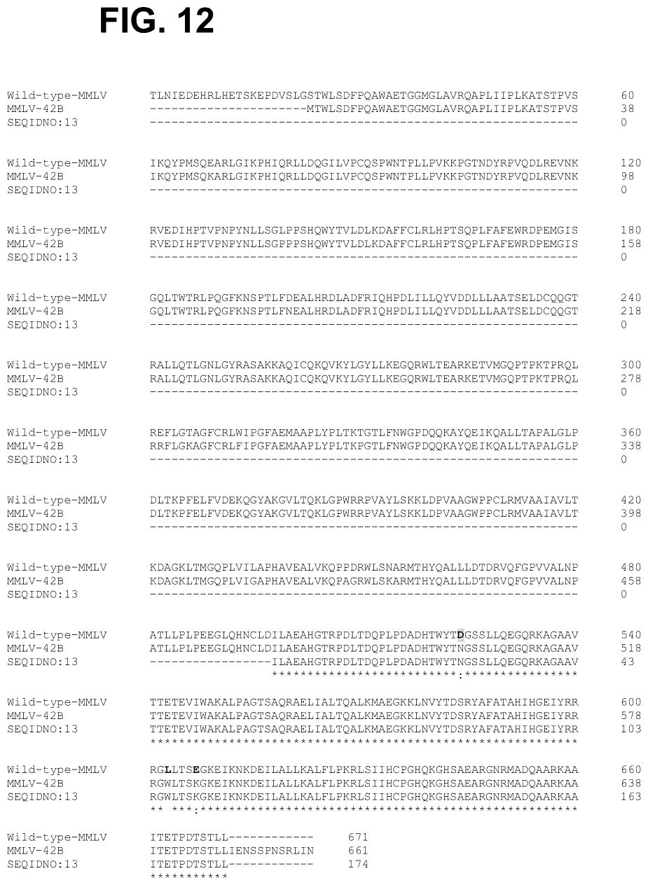

In the event the binding domain includes an enzymatic domain, the catalytic activity of the binding domain can be inactivated by mutation. In some embodiments of the chimeric ligase polypeptide described herein, the RNAseH polypeptide domain comprises a catalytically inactive domain. RNaseH enzymes have an active site centered on a conserved sequence motif of aspartate (“D”) and glutamate (“E”) residues, often referred to as the DEDD motif. In some embodiments, the catalytically inactive RNAseH includes at least one mutation to the DEDD motif. For example, in E. coli RNAseH, the mutation can be D10N mutation. In the RNAseH domain of a MMLV RT enzyme, the mutation can be D524N mutation (FIG. 12).

In some embodiments, the catalytically inactive RNAseH polypeptide domain is selected from the group consisting of Pyrococcus furiosus RNAseH, Pyrococcus horikoshi RNAseH, Pyrococcus woosii RNAseH, Bacillus sterothermophilus RNAse, Bacillus caldophilus RNAse, Thermococcus litoralis RNAseHII, Thermotoga neapolitana RNAse, Sulfolobus acidocaldarius RNAse, Thermoplasma acidophilum RNAseH, Thermus thermophilus RNAseH, Thermus thermophilus RNAseH I, Thermus thermophilus RNAseH II, Thermus thermophilus RNAseH III, Sulfobolus tokodaii RNAseH, Escherichia coli RNAseH (E. coli), and a viral reverse transcriptase RNAse domain.

In some embodiments, the catalytically inactive RNAseH polypeptide domain comprises a catalytically inactive E. coli RNAseH polypeptide. In some embodiments, the catalytically inactive E. coli RNAseH polypeptide comprises an E. coli RNAseH including a D10 mutation. In some embodiments, the catalytically inactive E. coli RNAseH polypeptide comprises an E. coli RNAseH including a D10N mutation.

In some embodiments, the catalytically inactive RNAseH polypeptide domain comprises a catalytically inactive Sulfobolus tokodaii RNAseH polypeptide. In some embodiments, the catalytically inactive Sulfobolus tokodaii RNAseH polypeptide comprises Sulfobolus tokodaii RNAseH including a D7 mutation. In some embodiments, the catalytically inactive Sulfobolus tokodaii RNAseH polypeptide comprises Sulfobolus tokodaii RNAseH including a D7N mutation.

In some embodiments, the catalytically inactive RNAseH polypeptide domain comprises a catalytically inactive viral reverse transcriptase RNAse domain. In some embodiments, the catalytically inactive RNAseH polypeptide domain enhances the binding of the chimeric ligase polypeptide to a RNA template, and DNA probe, or RNA-templated DNA probes.

In some embodiments, the catalytically inactive RNAseH polypeptide domain comprises a full-length E. coli RNAseH sequence. In some embodiments, the catalytically inactive RNAseH polypeptide domain comprises an amino acid sequence having at least about 90%, at least about 91%, at least about 92%, at least about 93%, at least about 94%, at least about 95%, at least about 96%, at least about 97%, at least about 98%, or at least about 99% sequence identity to SEQ ID NO: 31. In some embodiments, the catalytically inactive RNAseH polypeptide domain comprises an amino acid sequence set forth in SEQ ID NO: 31.

In some embodiments, the catalytically inactive RNAseH polypeptide domain comprises a full-length S. tokodaii RNAseH sequence. In some embodiments, the catalytically inactive RNAseH polypeptide domain comprises an amino acid sequence having at least about 90%, at least about 91%, at least about 92%, at least about 93%, at least about 94%, at least about 95%, at least about 96%, at least about 97%, at least about 98%, or at least about 99% sequence identity to SEQ ID NO: 33. In some embodiments, the catalytically inactive RNAseH polypeptide domain comprises an amino acid sequence set forth in SEQ ID NO: 33.

Some enzymes which act on RNA-DNA hybrid duplexes (like reverse transcriptase) have an RNAseH domain. Ribonuclease H (RNAseH) is an endoribonuclease that specifically hydrolyzes the phosphodiester bonds of RNA, which is annealed to DNA. RNAseH does not digest single or double-stranded DNA. Following formation of the cDNA, RNAseH can act on an RNA template/cDNA double-stranded complex to degrade the RNA. Because of RNAseH's particular activity, RNA nucleotides annealing to deoxynucleotides of the cDNA will be removed from the RNA template. Generally, the reverse transcriptase used an amplification reaction will have an RNAseH domain. As such, reverse transcriptase enzyme inherently have RNAseH activity that can cleave and degrade an RNA. See e.g., Oscorbin et al., Comput. Struct. Biotechnol. J. 2021; 19: 6315-6327.

Accordingly, in some embodiments of the chimeric ligase polypeptide described herein, the RNAseH domain is from a viral reverse transcriptase RNAseH domain. In some embodiments, the viral reverse transcriptase RNAseH domain is a Moloney Murine Leukemia Virus (MMLV) Reverse Transcriptase (RT) RNAseH domain or variant thereof.

In some embodiments, the MMLV RT RNAseH domain includes at least one catalytically inactivating mutation selected from: a D524 mutation, an E562 mutation, a D583 mutation, and a D653 mutation (residue numbering is with respect to wild-type MMLV reverse transcriptase). In some embodiments, the catalytically inactive MMLV RT RNAseH domain includes at least one of: (a) D524A, D524G, or D524N, (b) D528A, D528G, or D528N, and (c) D653A, D653D, D653H, D653N, or D653V. In some embodiments the catalytically inactive MMLV RT RNAseH domain includes a D524N mutation (FIG. 12).

In some embodiments, the catalytically inactive RNAseH polypeptide domain comprises a reverse transcriptase RNAseH domain from a MMLV comprising an amino acid an amino acid sequence having at least about 90%, at least about 91%, at least about 92%, at least about 93%, at least about 94%, at least about 95%, at least about 96%, at least about 97%, at least about 98%, or at least about 99% sequence identity to SEQ ID NO: 14.

In some embodiments, the catalytically inactive RNAseH polypeptide domain comprises the RNAseH domain of MMLV RT. In some embodiments of the chimeric ligase polypeptide described herein, the catalytically inactive RNAseH polypeptide domain comprises an amino acid sequence having at least about 90%, at least about 91%, at least about 92%, at least about 93%, at least about 94%, at least about 95%, at least about 96%, at least about 97%, at least about 98%, or at least about 99% sequence identity to SEQ ID NO: 13. In some embodiments, the catalytically inactive RNAseH polypeptide domain comprises an amino acid sequence set forth in SEQ ID NO: 13.

In some embodiments of the chimeric ligase polypeptide described herein, the catalytically inactive RNAseH polypeptide domain comprises a mutation in any of one of SEQ ID NO: 13, 31, or 33, and wherein the mutation is selected from a substitution, an insertion, a deletion, or any combination thereof.

### C. Chimeric Ligase Polypeptides

The present disclosure provides novel engineered ligase enzymes with multiple domains that allows the ligase to bind strongly to the RNA-DNA hybrid duplex during a ligation reaction thereby preventing the premature release of the ligase from the RNA-DNA hybrid duplex and the subsequent premature termination of the ligation reaction. The novel multidomain ligase enzymes were developed using a novel design. In the design, a ligase enzyme (e.g., a DNA ligase) is fused to an heterologous protein or a domain of a protein that can specifically bind RNA-DNA hybrid duplexes. The present disclosure demonstrates that such enzymes can be active for RNA templated DNA ligation and can exhibit improved properties compared to the single domain ligase.

The approach disclosed herein provides multidomain ligase enzymes with RNA-templated DNA ligation activity and improved properties including increased thermostability and decreased ligation junction bias. These properties have direct benefit to RNA-templated ligation dependent workflows, including increased probe design space and the potential to enable single nucleotide variation (SNV) workflows. The novel hybridization-based strategy described herein allows for fast, efficient, and low-sequence bias in RNA-templated DNA ligation during high-throughput genomic applications, such as for example in situ and single cell Turtle.

In some embodiments, the present disclosure provides a chimeric ligase polypeptide comprising, consisting essential of, or consisting of a ligase polypeptide domain comprising a T3 DNA ligase, a T4 RNA ligase, a T4 DNA ligase, a T7 DNA ligase, a HiFi Taq DNA ligase, a 9° N™ DNA Ligase, an E. coli DNA ligase, a Chlorella virus DNA ligase, a Paramecium bursaria Chlorella virus ligase, a Syngen Nebraska virus 5 ligase, or an Acanthocyslis turfacea chlorella virus ligase; and a catalytically inactive RNAseH polypeptide domain is selected from the group consisting of Pyrococcus furiosus RNAseH, Pyrococcus horikoshi RNAseH, Pyrococcus woosii RNAseH, Bacillus sterothermophilus RNAse, Bacillus caldophilus RNAse, Thermococcus litoralis RNAseH II, Thermotoga neapolitana RNAse, Sulfolobus acidocaldarius RNAse, Thermoplasma acidophilum RNAseH, Thermus thermophilus RNAseH, Thermus thermophilus RNAseHI, Thermus thermophilus RNAseHII, Thermus thermophilus RNAseHIII, Sulfobolus tokodaii RNAseH, Escherichia coli RNAseH, and a viral reverse transcriptase RNAse domain.

In some embodiments, the present disclosure provides a chimeric ligase polypeptide comprising, consisting essential of, or consisting of a ligase polypeptide domain comprising an amino acid sequence having at least about 90%, at least about 91%, at least about 92%, at least about 93%, at least about 94%, at least about 95%, at least about 96%, at least about 97%, at least about 98%, or at least about 99% sequence identity to any one of SEQ ID NO: 29, 36, 38, or 39; and a catalytically inactive RNAseH polypeptide domain comprising an amino acid sequence having at least about 90%, at least about 91%, at least about 92%, at least about 93%, at least about 94%, at least about 95%, at least about 96%, at least about 97%, at least about 98%, or at least about 99% sequence identity to SEQ ID NO: 13, 31, or 33.

In some embodiments, the chimeric ligase polypeptide comprises a ligase polypeptide domain comprising the amino acid sequence of SEQ ID NO: 36 and a catalytically inactive RNAseH polypeptide domain comprising the amino acid sequence of SEQ ID NO: 13, 31, or 33.

In some embodiments, the chimeric ligase polypeptide comprises a ligase polypeptide domain comprising the amino acid sequence of SEQ ID NO: 36 and a catalytically inactive RNAseH polypeptide domain comprising the amino acid sequence of SEQ ID NO: 13, 31, or 33. In some embodiments, the chimeric ligase polypeptide comprises a ligase polypeptide domain comprising the amino acid sequence of SEQ ID NO: 38 and a catalytically inactive RNAseH polypeptide domain comprising the amino acid sequence of SEQ ID NO: 13, 31, or 33. In some embodiments, the chimeric ligase polypeptide comprises a ligase polypeptide domain comprising the amino acid sequence of SEQ ID NO: 39 and a catalytically inactive RNAseH polypeptide domain comprising the amino acid sequence of SEQ ID NO: 13, 31, or 33.

One aspect of the present disclosure provides a chimeric ligase polypeptide comprising an amino acid sequence that is at least 90%, at least 92%, at least 95%, at least 97%, at least 98%, or at least 99% identical to SEQ ID NO: 2.

SEQ ID NO: 1: LigAT_C-RNAseH-
Ecoli nucleic acid sequence.
LigAT_C-RNAseH-Ecoli comprises
LigAT ligase fused to the RNAseH of E. coli.
atggctattcagaagccgttactggcggccagcctgaagaaaatg
tctgttgacaacttgactttcccggtctacgcaacgccgaaactg
gacggtatccgtgcgctgaagattgacggcaccctggtgtcgcgt
acgttcaaaccgatccgcaatactaccatctccaaggtcctggcg
agcttgctgccggacggcagcgatggtgagatcttgtctggtaag
acctttcaggatagcaccagcaccgtgatgacgaccgatgcgggt
atcggtagcgataccaccttcttctggtttgattacgttaaagac
gacccggataagggttacctggaccgtatcgctgacatgaaaacc
ttcgtcgaccagcacccggaaatcctgaaagacagctgcgtgacg
atcgtcccgctgttcccgaagaaaattgacacgccagaagaactg
catgtgtttgagaaatggtgtctggaccagggttttgagggcgtt
atggtgcgtacggccggtggcaagtataagtttggtcgcagcacc
gagaaagaacaaatcctggttaagattaaacagtttgaagatgat
gaagctgttgtgatcggtgtgagcgcgctgcaaaccaatacgaac
gataagaaactgaatcagctgggtgagatgcgtcgtactagccac
caagacggcaaagttgacctggacatgctgggtgcgcttgacgtg
gactggaatggtattcgtttcagcatcggtacgggctttgataaa
gacacccgcgaagatctctggaaacaacgcgacagcatcgtgggt
aaagtcgttaagttcaagtatttctcccagggtattaagaccgct
ccgcgttttcctgtgtttctgggttttcgtgacgagaacgatatg
GGTGGCAGCGGTGGCAGCATGCTGAAGCAGGTGGAAATCTTTACA
AACGGTTCTTGTCTGGGAAATCCTGGACCCGGAGGATATGGTGCA
ATTTTGCGTTATCGCGGACGCGAAAAGACATTTTCAGCGGGATAT
ACTCGTACAACGAACAACCGCATGGAACTTATGGCCGCAATCGTG
GCACTTGAGGCGCTGAAGGAACATTGCGAAGTGATCCTGTCTACA
GATAGTCAATATGTGCGCCAAGGAATTACGCAGTGGATCCATAAT
TGGAAGAAGCGTGGCTGGAAGACCGCAGACAAAAAACCAGTGAAA
AACGTAGACTTATGGCAACGTCTGGATGCTGCTCTGGGACAACAC
CAAATCAAATGGGAATGGGTGAAGGGACATGCCGGACATCCCGAG
AATGAGCGTTGTGACGAATTAGCGCGTGCCGCCGCAATGAATCCT
ACGCTGGAAGACACCGGTTATCAGGTGGAGGTTTAA
SEQ ID NO: 2 sets forth the amino acid sequence
of LigAT_C-RNAseH-Ecoli.
maiqkpllaaslkkmsvdnltfpvyatpkldgiralkidgtlvsr
tfkpirnttiskvlasllpdgsdgeilsgktfqdststvmttdag
igsdttffwfdyvkddpdkgyldriadmktfvdqhpeilkdscvt
ivplfpkkidtpeelhvfekwcldqgfegvmvrtaggkykfgrst
ekeqilvkikqfeddeavvigvsalqtntndkklnqlgemrrtsh
qdgkvdldmlgaldvdwngirfsigtgfdkdtredlwkqrdsivg
kvvkfkyfsqgiktaprfpvflgfrdendmGGSGGSMLKQVEIFT
NGSCLGNPGPGGYGAILRYRGREKTFSAGYTRTTNNRMELMAAIV
ALEALKEHCEVILSTDSQYVRQGITQWIHNWKKRGWKTADKKPVK
NVDLWQRLDAALGQHQIKWEWVKGHAGHPENERCDELARAAAMNP
TLEDTGYQVEV
LigAT-lowercase; linker-underlined uppercase;
catalytically inactive RNAseH from E. coli
(with D524N mutation)-uppercase bold.
In some embodiments, the chimeric
ligase polypeptide comprises the amino acid
sequence of SEQ ID NO: 2 or variant thereof.

In some embodiments, the chimeric ligase polypeptide comprises an amino acid sequence that is at least 90%, at least 92%, at least 95%, at least 97%, at least 98%, or at least 99% identical to SEQ ID NO: 4.

-LigAT_N-RNAseH-Ecoli nucleic acid sequence. LigAT_N-
RNAseH-Ecoli comprises the LigAT ligase fused to the RNAseH from E. coli.
SEQ ID NO: 3
ATGCTGAAGCAGGTGGAAATCTTTACAAACGGTTCTTGTCTGGGAAAT
CCTGGACCCGGAGGATATGGTGCAATTTTGCGTTATCGCGGACGCGAAAAG
ACATTTTCAGCGGGATATACTCGTACAACGAACAACCGCATGGAACTTATGG
CCGCAATCGTGGCACTTGAGGCGCTGAAGGAACATTGCGAAGTGATCCTGT
CTACAGATAGTCAATATGTGCGCCAAGGAATTACGCAGTGGATCCATAATTG
GAAGAAGCGTGGCTGGAAGACCGCAGACAAAAAACCAGTGAAAAACGTAGA
CTTATGGCAACGTCTGGATGCTGCTCTGGGACAACACCAAATCAAATGGGA
ATGGGTGAAGGGACATGCCGGACATCCCGAGAATGAGCGTTGTGACGAATT
AGCGCGTGCCGCCGCAATGAATCCTACGCTGGAAGACACCGGTTATCAGGT
GGAGGTTGGTTCAGGGGGTTCCGGTatggctattcagaagccgttactggggccagcctgaagaaaatgtc
tgttgacaacttgactttcccggtctacgcaacgccgaaactggacggtatccgtgcgctgaagattgacggcaccctggtgtcgcgta
cgttcaaaccgatccgcaatactaccatctccaaggtcctggcgagcttgctgccggacggcagcgatggtgagatcttgtctggtaag
acctttcaggatagcaccagcaccgtgatgacgaccgatgcgggtatcggtagcgataccaccttcttctggtttgattacgttaaagac
gacccggataagggttacctggaccgtatcgctgacatgaaaaccttcgtcgaccagcacccggaaatcctgaaagacagctgcgtg
acgatcgtcccgctgttcccgaagaaaattgacacgccagaagaactgcatgtgtttgagaaatggtgtctggaccagggttttgaggg
cgttatggtgcgtacggccggtggcaagtataagtttggtcgcagcaccgagaaagaacaaatcctggttaagattaaacagtttgaag
atgatgaagctgttgtgatcggtgtgagcgcgctgcaaaccaatacgaacgataagaaactgaatcagctgggtgagatgcgtcgtac
tagccaccaagacggcaaagttgacctggacatgctgggtgcgcttgacgtggactggaatggtattcgtttcagcatcggtacgggc
tttgataaagacacccgcgaagatctctggaaacaacgcgacagcatcgtgggtaaagtcgttaagttcaagtatttctcccagggtatt
aagaccgctccgcgttttcctgtgtttctgggttttcgtgacgagaacgatatgtaa
sets forth the amino acid sequence of LigAT_N-RNAseH-Ecoli.
SEQ ID NO: 4
MLKQVEIFTNGSCLGNPGPGGYGAILRYRGREKTFSAGYTRTTNNRMEL
MAAIVALEALKEHCEVILSTDSQYVRQGITQWIHNWKKRGWKTADKKPVKNV
DLWQRLDAALGQHQIKWEWVKGHAGHPENERCDELARAAAMNPTLEDTGYQ
VEVGSGGSGmaiqkpllaaslkkmsvdnltfpvyatpkldgiralkidgtlvsrtfkpirnttiskvlasllpdgsdgeilsgkt
fqdststvmttdagigsdttffwfdyvkddpdkgyldriadmktfvdqhpeilkdscvtivplfpkkidtpeelhvfekwcldqgf
egvmvrtaggkykfgrstekeqilvkikqfeddeavvigvsalqtntndkklnqlgemrrtshqdgkvdldmlgaldvdwngir
fsigtgfdkdtredlwkqrdsivgkvvkfkyfsqgiktaprfpvflgfrdendm

LigAT-lowercase; linker-underlined uppercase; catalytically inactive RNAseH from E. coli (with D10N mutation)-uppercase bold. In some embodiments, the chimeric ligase polypeptide comprises the amino acid sequence of SEQ ID NO: 4.

In some embodiments, the chimeric ligase polypeptide comprises an amino acid sequence that is at least 90%, at least 92%, at least 95%, at least 97%, at least 98%, or at least 99% identical to SEQ ID NO: 6.

-LigAT_C-RNAseH-Sto nucleic acid sequence. LigAT_C-RNAseH-
Sto includes LigAT fused to the RNAseHI of Sulfobolus tokodaii (Sto).
SEQ ID NO: 5
atggctattcagaagccgttactggcggccagcctgaagaaaatgtctgttgacaacttgactttcccggtctacgcaacgc
cgaaactggacggtatccgtgcgctgaagattgacggcaccctggtgtcgcgtacgttcaaaccgatccgcaatactaccatctccaa
ggtcctggcgagcttgctgccggacggcagcgatggtgagatcttgtctggtaagacctttcaggatagcaccagcaccgtgatgacg
accgatgcgggtatcggtagcgataccaccttcttctggtttgattacgttaaagacgacccggataagggttacctggaccgtatcgct
gacatgaaaaccttcgtcgaccagcacccggaaatcctgaaagacagctgcgtgacgatcgtcccgctgttcccgaagaaaattgac
acgccagaagaactgcatgtgtttgagaaatggtgtctggaccagggttttgagggcgttatggtgcgtacggccggtggcaagtata
agtttggtcgcagcaccgagaaagaacaaatcctggttaagattaaacagtttgaagatgatgaagctgttgtgatcggtgtgagcgcg
ctgcaaaccaatacgaacgataagaaactgaatcagctgggtgagatgcgtcgtactagccaccaagacggcaaagttgacctggac
atgctgggtgcgcttgacgtggactggaatggtattcgtttcagcatcggtacgggctttgataaagacacccgcgaagatctctggaa
acaacgcgacagcatcgtgggtaaagtcgttaagttcaagtatttctcccagggtattaagaccgctccgcgttttcctgtgtttctgggttt
tcgtgacgagaacgatatgGGTGGCAGCGGTGGCAGCATGATCATTGGTTACTTCAACG
GGCTTTGCGAACCTAAAAACCCCGGCGGTATTGCGACCTTTGGCTTCGTCAT
TTACCTGGATAACCGCAAAATCGAGGGCTATGGTTTAGCCGAGAAGCCATTC
TCTATCAACTCCACCAATAATGTTGCCGAGTATTCCGGTTTGATCTGCCTTA
TGGAGACAATGTTACGCTTAGGAATTAGTAGTCCCATTATTAAAGGTGACTC
ACAATTGGTTATCAAACAGATGAATGGCGAGTACAAGGTCAAAGCGAAACG
TATTATCCCTTTATACGAGAAAGCTATTGAATTGAAAAAGAAGTTGAATGCC
ACCCTTATTTGGGTGCCTCGCGAGGAGAATAAGGAGGCTGATCGTTTGTCTC
GTGTCGCCTATGAGTTGGTGCGTCGCGGCAAATTGCGTGACATTGGGTGCA
TTATTCTGACGTAA
sets forth the amino acid sequence of LigAT_C-RNAseH-Sto.
SEQ ID NO: 6
maiqkpllaaslkkmsvdnltfpvyatpkldgiralkidgtlvsrtfkpirnttiskvlasllpdgsdgeilsgktfqdststv
mttdagigsdttffwfdyvkddpdkgyldriadmktfvdqhpeilkdscvtivplfpkkidtpeelhvfekwcldqgfegvmvrt
aggkykfgrstekeqilvkikqfeddeavvigvsalqtntndkklnqlgemrrtshqdgkvdldmlgaldvdwngirfsigtgfd
kdtredlwkqrdsivgkvvkfkyfsqgiktaprfpvflgfrdendmGGSGGSMIIGYFNGLCEPKNPGGIA
TFGFVIYLDNRKIEGYGLAEKPFSINSTNNVAEYSGLICLMETMLRLGISSPIIKG
DSQLVIKQMNGEYKVKAKRIIPLYEKAIELKKKLNATLIWVPREENKEADRLSR
VAYELVRRGKLRDIGCIILT

LigAT-lowercase; LINKER-underlined uppercase; Catalytically inactive RNAseHI from Sulfobolus tokodaii (with D7N mutation)-uppercase bold. In some embodiments, the chimeric ligase polypeptide comprises the amino acid sequence of SEQ ID NO: 6.

In some embodiments, the chimeric ligase polypeptide comprises an amino acid sequence that is at least 90%, at least 92%, at least 95%, at least 97%, at least 98%, or at least 99% identical to SEQ ID NO: 8.

SEQ ID NO: 7: LigAT_C-RNAseH-RT nucleic acid sequence. LigAT_C-RNAseH-
RT comprises LigAT ligase fused to a catalytically inactive RNAseH domain from a reverse
transcriptase enzyme variant (MMLV variant).
atggctattcagaagccgttactggcggccagcctgaagaaaatgtctgttgacaacttgactttcccggtctacgcaacgc
cgaaactggacggtatccgtgcgctgaagattgacggcaccctggtgtcgcgtacgttcaaaccgatccgcaatactaccatctccaa
ggtcctggcgagcttgctgccggacggcagcgatggtgagatcttgtctggtaagacctttcaggatagcaccagcaccgtgatgacg
accgatgcgggtatcggtagcgataccaccttcttctggtttgattacgttaaagacgacccggataagggttacctggaccgtatcgct
gacatgaaaaccttcgtcgaccagcacccggaaatcctgaaagacagctgcgtgacgatcgtcccgctgttcccgaagaaaattgac
acgccagaagaactgcatgtgtttgagaaatggtgtctggaccagggttttgagggcgttatggtgcgtacggccggtggcaagtata
agtttggtcgcagcaccgagaaagaacaaatcctggttaagattaaacagtttgaagatgatgaagctgttgtgatcggtgtgagcgcg
ctgcaaaccaatacgaacgataagaaactgaatcagctgggtgagatgcgtcgtactagccaccaagacggcaaagttgacctggac
atgctgggtgcgcttgacgtggactggaatggtattcgtttcagcatcggtacgggctttgataaagacacccgcgaagatctctggaa
acaacgcgacagcatcgtgggtaaagtcgttaagttcaagtatttctcccagggtattaagaccgctccgcgttttcctgtgtttctgggttt
tcgtgacgagaacgatatgGGTGGCAGCGGTGGCAGCATCCTGGCAGAGGCGCACGGCAC
TCGCCCGGATCTGACGGACCAGCCGCTGCCGGACGCCGATCATACCTGGTA
TACGAATGGTAGCAGCCTGTTGCAAGAGGGTCAGCGTAAGGCCGGTGCCGC
GGTCACCACCGAGACTGAAGTGATTTGGGCTAAAGCATTGCCTGCGGGTAC
CAGCGCGCAGCGTGCCGAGCTGATCGCACTGACCCAAGCGCTGAAAATGGC
TGAGGGTAAGAAACTGAATGTGTACACGGATAGCCGTTATGCCTTTGCGAC
CGCCCACATTCACGGCGAGATCTATCGCCGTCGCGGCTGGCTGACGTCCAA
AGGCAAAGAGATCAAGAATAAAGACGAAATTCTGGCGCTGCTGAAAGCGCT
GTTCCTGCCGAAACGTCTGTCGATCATCCATTGCCCGGGTCACCAGAAAGG
CCACAGCGCAGAGGCGCGTGGTAATCGCATGGCTGACCAGGCTGCGCGTAA
AGCCGCAATTACCGAAACCCCGGACACCAGCACGCTGCTGTAA
sets forth the amino acid sequence of LigAT_C-RNAseH-RT.
SEQ ID NO: 8
maiqkpllaaslkkmsvdnltfpvyatpkldgiralkidgtlvsrtfkpirnttiskvlasllpdgsdgeilsgktfqdststv
mttdagigsdttffwfdyvkddpdkgyldriadmktfvdqhpeilkdscvtivplfpkkidtpeelhvfekwcldqgfegvmvrt
aggkykfgrstekeqilvkikqfeddeavvigvsalqtntndkklnqlgemrrtshqdgkvdldmlgaldvdwngirfsigtgfd
kdtredlwkqrdsivgkvvkfkyfsqgiktaprfpvflgfrdendmGGSGGSILAEAHGTRPDLTDQPLPD
ADHTWYTNGSSLLQEGQRKAGAAVTTETEVIWAKALPAGTSAQRAELIALTQA
LKMAEGKKLNVYTDSRYAFATAHIHGEIYRRRGWLTSKGKEIKNKDEILALLK
ALFLPKRLSIIHCPGHQKGHSAEARGNRMADQAARKAAITETPDTSTLL

LigAT-lowercase; LINKER-underlined uppercase; Catalytically inactive RNAseH domain from an MMLV reverse transcriptase enzyme-uppercase bold. In some embodiments, the chimeric ligase polypeptide comprises the amino acid sequence of SEQ ID NO: 8.

In some embodiments, the chimeric ligase polypeptide comprises an amino acid sequence that is at least 90%, at least 92%, at least 95%, at least 97%, at least 98%, or at least 99% identical to SEQ ID NO: 10.

SEQ ID NO: 9: T4-DNA-ligase_RNAseH-RT nucleic acid sequence. T4-DNA-
ligase_RNAseH-MMLV RT includes a T4 DNA ligase operably linked to a catalytically
inactive RNAseH domain from an MMLV reverse transcriptase enzyme.
atgatcctgaagattttgaacgagatcgctagcatcgggagtacaaaacaaaagcaggcgatcttagagaaaaacaaaga
caacgagctgttgaaacgtgtgtatcgtttaacctatagccgtggtcttcagtactacatcaaaaagtggccgaagccaggaattgccac
acaatctttcggcatgcttactttaactgacatgctggatttcatcgaatttaccctggcaacgcgtaaattgaccgggaatgctgctatcg
aagagctgactggttatattaccgacggaaaaaaagacgacgtcgaagttttgcgccgcgtcatgatgcgtgatctggaatgcggcgc
gtcggtctcaattgctaacaaagtctggcctgggctgattccagagcagccccaaatgcttgcaagcagttacgatgagaagggtatta
acaaaaacattaagtttcctgcattcgctcaactgaaggccgatggtgcccgctgttttgcggaagtccgtggcgacgaactggacgac
gtgcgccttctgtctcgcgctgggaacgaatatttgggcttggatcttcttaaggaagaattgattaagatgaccgcggaggcccgtcag
attcatcccgaaggggtgctgatcgacggcgaattggtatatcatgagcaggtaaaaaaagagcccgagggactggactttctttttga
cgcatacccggaaaactccaaggcgaaggagttcgcggaggttgctgaatcacgtacagcctccaatgggatcgccaacaaatcgct
taagggaaccatttccgaaaaggaggcacagtgcatgaaattccaggtctgggactacgtccccttagtagaaatctactccttgcctg
ctttccgtttgaaatatgatgtccgtttctccaaattggagcagatgacttctgggtatgacaaggttatcttaatcgaaaatcaggtcgtga
ataacctggacgaagcaaaagtaatctataaaaagtatatcgaccagggattagaaggcattattctgaaaaacatcgacggcttatgg
gagaacgcgcgttcgaagaacttatataaatttaaggaagttatcgatgttgatcttaagatcgttgggatctacccacatcgtaaagatc
caaccaagggggaggattcattttagagtccgaatgcggcaagattaaggtaaacgcgggttctggccttaaggataaggccgggg
taaagtctcacgagttggatcgcacacgtattatggaaaatcagaattactatatcggcaaaatcttagaatgcgaatgtaatgggtggtt
gaagagcgatggacgcaccgattatgtcaaattatttctgccgatcgccattcgcctgcgcgaggataaaacgaaagcgaacactttcg
aggatgtgttcggcgactttcacgaagtcactggcctgGGTGGCAGCGGTGGCAGCATCCTGGCAGAG
GCGCACGGCACTCGCCCGGATCTGACGGACCAGCCGCTGCCGGACGCCGAT
CATACCTGGTATACGAATGGTAGCAGCCTGTTGCAAGAGGGTCAGCGTAAG
GCCGGTGCCGCGGTCACCACCGAGACTGAAGTGATTTGGGCTAAAGCATTG
CCTGCGGGTACCAGCGCGCAGCGTGCCGAGCTGATCGCACTGACCCAAGCG
CTGAAAATGGCTGAGGGTAAGAAACTGAATGTGTACACGGATAGCCGTTAT
GCCTTTGCGACCGCCCACATTCACGGCGAGATCTATCGCCGTCGCGGCTGG
CTGACGTCCAAAGGCAAAGAGATCAAGAATAAAGACGAAATTCTGGCGCTG
CTGAAAGCGCTGTTCCTGCCGAAACGTCTGTCGATCATCCATTGCCCGGGTC
ACCAGAAAGGCCACAGCGCAGAGGCGCGTGGTAATCGCATGGCTGACCAGG
CTGCGCGTAAAGCCGCAATTACCGAAACCCCGGACACCAGCACGCTGCTGT
AA
sets forth the amino acid sequence of T4-DNA-ligase_RNAseH-RT.
SEQ ID NO: 10
milkilneiasigstkqkqaileknkdnellkrvyrltysrglqyyikkwpkpgiatqsfgmltltdmldfieftlatrkltg
naaieeltgyitdgkkddvevlrrvmmrdlecgasvsiankvwpglipeqpqmlassydekginknikfpafaqlkadgarcfa
evrgdelddvrllsragneylgldllkeelikmtaearqihpegvlidgelvyheqvkkepegldflfdaypenskakefaevaesrt
asngiankslkgtisekeaqcmkfqvwdyvplveiyslpafrlkydvrfskleqmtsgydkvilienqvvnnldeakviykkyid
qglegiilknidglwenarsknlykfkevidvdlkivgiyphrkdptkaggfilesecgkikvnagsglkdkagvksheldrtrim
enqnyyigkilececngwlksdgrtdyvklflpiairlredktkantfedvfgdfhevtglGGSGGSILAEAHGTRPD
LTDQPLPDADHTWYTNGSSLLQEGQRKAGAAVTTETEVIWAKALPAGTSAQRA
ELIALTQALKMAEGKKLNVYTDSRYAFATAHIHGEIYRRRGWLTSKGKEIKNK
DEILALLKALFLPKRLSIIHCPGHQKGHSAEARGNRMADQAARKAAITETPDTST
LL

T4 DNA ligase-lowercase; LINKER-underlined uppercase; Catalytically inactive RNAseH domain from an MMLV reverse transcriptase enzyme-uppercase bold.

In some embodiments, the chimeric ligase polypeptide comprises an amino acid sequence that is at least 90%, at least 92%, at least 95%, at least 97%, at least 98%, or at least 99% identical to SEQ ID NO: 12

SEQ ID NO: 11: T4-RNA-ligase2_RNAseH-RT nucleic acid sequence. T4-RNA-
ligase2_RNAseH-RT comprises a T4 RNA ligase2 operably linked to a catalytically inactive
RNAseH domain from a variant MMLV reverse transcriptase enzyme.
atgtttaaaaagtatagtagcctggaaaatcactataatagcaaatttattgagaaattgtactcattaggtcttaccggaggag
agtgggtagcgcgtgagaaaatccatgggactaacttttcacttattatcgagcgtgataaggtgacttgcgctaaacgcactgggccc
atcctgcccgcagaagacttcttcggttatgagattattctgaaaaactatgccgactcgatcaaggcagttcaagacatcatggaaacg
tcggcagtagtctcctaccaggtatttggcgaattcgcaggtcctggcattcagaagaatgtagactactgtgataaagatttctacgtctt
cgacatcattgtgaccactgagtcgggagatgttacctatgtagacgattatatgatggagagtttctgtaacaccttcaaatttaagatgg
caccgttattaggtcgcggtaaatttgaggagctgattaagttaccaaatgacttagacagcgttgtacaggattacaatttcacagtcgat
catgccggtttagttgacgctaacaaatgtgtatggaacgctgaggcaaaaggcgaagtgtttaccgccgaaggttatgttctgaaacct
tgttatccaagctggttgcgtaacggcaaccgtgtcgcgatcaaatgcaagaactctaaattcagtgaaaagaaaaaaagcgataagc
cgattaaggctaaggtagagttatccgaggctgacaataagttagtgggaatcttggcgtgttatgtgacgctgaatcgcgtgaacaatg
ttatctctaaaattggagaaatcggcccaaaggactttggaaaggtcatgggacttacggtccaggatattctggaggaaacgtctcgc
gagggtattaccttaacgcaggcagacaatccgtcccttatcaaaaaagaattggtcaaaatggtccaggacgtgctgcgccctgcgt
ggattgagcttgtgagcGGTGGCAGCGGTGGCAGCATCCTGGCAGAGGCGCACGGCACT
CGCCCGGATCTGACGGACCAGCCGCTGCCGGACGCCGATCATACCTGGTAT
ACGAATGGTAGCAGCCTGTTGCAAGAGGGTCAGCGTAAGGCCGGTGCCGCG
GTCACCACCGAGACTGAAGTGATTTGGGCTAAAGCATTGCCTGCGGGTACC
AGCGCGCAGCGTGCCGAGCTGATCGCACTGACCCAAGCGCTGAAAATGGCT
GAGGGTAAGAAACTGAATGTGTACACGGATAGCCGTTATGCCTTTGCGACC
GCCCACATTCACGGCGAGATCTATCGCCGTCGCGGCTGGCTGACGTCCAAA
GGCAAAGAGATCAAGAATAAAGACGAAATTCTGGCGCTGCTGAAAGCGCTG
TTCCTGCCGAAACGTCTGTCGATCATCCATTGCCCGGGTCACCAGAAAGGCC
ACAGCGCAGAGGCGCGTGGTAATCGCATGGCTGACCAGGCTGCGCGTAAAG
CCGCAATTACCGAAACCCCGGACACCAGCACGCTGCTGTAA
sets forth the amino acid sequence of T4-RNA-ligase2_RNAseH-RT.
SEQ ID NO: 12
mfkkysslenhynskfieklyslgltggewvarekihgtnfsliierdkvtcakrtgpilpaedffgyeiilknyadsikav
qdimetsavvsyqvfgefagpgiqknvdycdkdfyvfdiivttesgdvtyvddymmesfcntfkfkmapllgrgkfeeliklpn
dldsvvqdynftvdhaglvdankcvwnaeakgevftaegyvlkpcypswlrngnrvaikcknskfsekkksdkpikakvelse
adnklvgilacyvtlnrvnnviskigeigpkdfgkvmgltvqdileetsregitltqadnpslikkelvkmvqdvlrpawielvsG
GSGGSILAEAHGTRPDLTDQPLPDADHTWYTNGSSLLQEGQRKAGAAVTTETEV
IWAKALPAGTSAQRAELIALTQALKMAEGKKLNVYTDSRYAFATAHIHGEIYRR
RGWLTSKGKEIKNKDEILALLKALFLPKRLSIIHCPGHQKGHSAEARGNRMAD
QAARKAAITETPDTSTLL

T4 RNA ligase2-lowercase; LINKER-underlined uppercase; Catalytically inactive RNAseH domain from a variant MMLV reverse transcriptase enzyme-uppercase bold. In some embodiments, the chimeric ligase polypeptide comprises the amino acid sequence of SEQ ID NO: 12.

The amino acid sequence of RT-RNAseH domain is set forth in SEQ ID NO: 13. This corresponds to the region from 1498 to L671 of a full length wild-type MMLV RT amino acid sequence (inclusive) (FIG. 12).

ILAEAHGTRPDLTDQPLPDADHTWYTNGSSLLQEGQRKAGAAVTTETEV
IWAKALPAGTSAQRAELIALTQALKMAEGKKLNVYTDSRYAFATAHIHG
EIYRRRGWLTSKGKEIKNKDEILALLKALFLPKRLSIIHCPGHQKGHSA
EARGNRMADQAARKAAITETPDTSTLL

As shown in the Examples, chimeric enzymes comprising an amino acid sequence described herein or encoded by the nucleic acid sequence described herein were successfully purified. These chimeric ligases were tested using in vitro assays (e.g., capillary electrophoresis; CE assay) and in an in situ workflow. These assays demonstrated that these chimeric ligase polypeptides were active ligases for RNA-templated DNA ligation and had improved properties when compared to a wild-type ligase (LigAT) or a recombinant ligase comprising a ligase (e.g., LigAT) fused to single stranded DNA binding protein.

In some embodiments, the chimeric ligase enzyme described herein is an active ligase for RNA-templated DNA ligation. In some embodiments, the chimeric ligase enzyme described herein had improved properties when compared to the corresponding wild-type ligase (e.g., LigAT) or a recombinant ligase comprising a ligase (e.g., LigAT) fused to single stranded DNA binding protein.

In some embodiments, the chimeric ligase enzyme described herein had improved properties when compared to a wild-type ligase (LigAT) or a recombinant ligase comprising a ligase (e.g., LigAT) fused to single stranded DNA binding protein (e.g., HU). In some embodiments, the chimeric ligase enzyme described herein had improved ligation properties when compared to a recombinant ligase-HU fusion protein. In some embodiments, the chimeric ligase enzyme described herein had improved properties when compared to a recombinant LigAT-HU fusion. In some embodiments, the chimeric ligase enzyme described herein exhibits decreased ligation junction bias when compared to a wild-type ligase (LigAT) or a recombinant ligase comprising LigAT fused to single stranded DNA binding protein. In some embodiments, the chimeric ligase enzyme described herein exhibits decreased ligation junction bias when compared to LigAT-HU.

In some embodiments, the chimeric ligase enzymes described herein show improved thermostability when compared to a wild-type ligase (e.g., LigAT, T4 DNA ligase, or T4 RNA ligase). In some embodiments, the chimeric ligase enzymes described herein show improved thermostability when compared to a recombinant ligase comprising LigAT fused to single stranded DNA binding protein. In some embodiments, the chimeric ligase enzymes described herein show improved thermostability when compared to a Ligase-HU fusion protein. In some embodiments, the chimeric ligase enzymes described herein show improved thermostability when compared to a LigAT-HU.

In some embodiments, the chimeric ligase polypeptide described herein exhibits increased RNA-templated DNA ligation, enhanced ligase activity, improved thermostability, enhanced thermoreactivity, decreased ligation junction bias, or any combination thereof when compared to a control. In some embodiments, the control is not fused to an RNAseH domain. In some embodiments, the control comprises the amino acid sequence of SEQ ID NO: 40.

### D. Thermostability

As used herein, the term “thermostable” generally refers to an enzyme, such as a chimeric ligase polypeptide described herein, which retains a greater percentage or amount of its activity after a heat treatment than is retained by the same enzyme having wild type thermostability, after an identical treatment. Thus, a chimeric ligase polypeptide having increased/enhanced thermostability may be defined as a chimeric ligase polypeptide having any increase in thermostability, preferably from about 1.2 to about 10,000 fold, from about 1.5 to about 10,000 fold, from about 2 to about 5,000 fold, or from about 2 to about 2000 fold, or any value in between these amounts, and retention of activity after a heat treatment sufficient to cause a reduction in the activity of a ligase that is wild type for thermostability. In other aspects of the disclosure, the increase in thermostability can be greater than about 5 fold, greater than about 10 fold, greater than about 50 fold, greater than about 100 fold, greater than about 500 fold, or greater than about 1000 fold.

In some embodiments, the chimeric ligase polypeptide described herein exhibits improved thermostability or thermoreactivity. In that embodiment, the increase in thermoreactivity, or thermostability is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to a wild-type ligase or a control ligase not fused to an RNAseH domain. In some embodiments, the control ligase not fused to an RNAseH domain is a recombinant ligase. In some embodiments, the control ligase not fused to an RNAseH domain is a recombinant ligase comprising the amino acid of SEQ ID NO: 40.

During ligation, the reaction mixture in certain embodiments may be maintained at a temperature ranging from about 4° C. to about 105° C., about 4 to about 80° C., such as about 10 to about 70° C., about 15 to about 60° C., typically such as from about 20° C. to about 37° C. for a period of time ranging from about 5 seconds to about 16 hours, such as from about 1 minute to about 1 hour. In yet other embodiments, the reaction mixture may be maintained at a temperature ranging from about 35° C. to about 45° C., such as from about 37° C. to about 42° C., e.g., at or about 38° C., 39° C., 40° C. or 41° C., for a period of time ranging from about 5 seconds to about 16 hours, such as from about 1 minute to about 1 hour, including from about 2 minutes to about 8 hours.

To determine the thermostability of the chimeric ligase polypeptide of the present disclosure, the chimeric ligase polypeptide can be compared to the corresponding wildtype ligase or a variant thereof (e.g., SEQ ID NO: 40) to determine the relative enhancement or increase in thermostability. For example, after a heat treatment at 55° C. for 60 minutes, the chimeric ligase polypeptide may retain approximately 90% of the activity present before the heat treatment, whereas wild type chimeric ligase polypeptide or variant (e.g., SEQ ID NO: 40) may retain less than 5% of its original activity. Likewise, after a heat treatment at 55° C. for 120 minutes, the chimeric ligase polypeptide may retain approximately 80% of its original activity, whereas wild type chimeric ligase polypeptide or variant (e.g., SEQ ID NO: 40) may have no measurable activity.

Similarly, after a heat treatment at 55° C. for 120 minutes, the e chimeric ligase polypeptide may retain approximately 50%, approximately 55%, approximately 60%, approximately 65%, approximately 70%, approximately 75%, approximately 80%, approximately 85%, approximately 90%, or approximately 95% of its original activity, whereas wild type ligase or a ligase variant (e.g., SEQ ID NO: 40) may have no measurable activity or may retain 20%, 15%, 10%, or none of its original activity. Examples of conditions which may be used to measure thermostability of an enzyme such as chimeric ligase polypeptide are set out in further detail below and in the Examples (FIGS. 5A-E).

In some embodiments, the chimeric ligase polypeptide is active at a temperature of at least 55° C., at least 60° C., or at least 65° C. In some embodiments, the chimeric ligase polypeptide produces the same ligation product yield at a reaction temperature of at least 550 C as a ligation product yield produced as at a reaction temperature of 37° C. In some embodiments, the chimeric ligase polypeptide produces at least about 2-fold to at least about 15-fold more ligation product at 37° C., 43° C., 48° C., 52° C., or 55° C. when compared to a wild-type ligase or a control ligase not fused to an RNAseH domain. In some embodiments, the control ligase not fused to an RNAseH domain is a recombinant ligase. In some embodiments, the control ligase not fused to an RNAseH domain is a recombinant ligase comprising the amino acid of SEQ ID NO: 40.

### E. Linkers

In some embodiments of the chimeric ligase polypeptide described herein, the ligase polypeptide domain and the catalytically inactive RNAseH polypeptide domain are operably linked by a linker. The linker can be any length (e.g., 1 to >20 amino acids). The linker can also comprise numerous combinations of amino acids. An exemplified linker chosen for the constructs shown in Table 3 and the examples comprises 6 amino acids (SEQ ID NO: 17) or is encoded by SEQ ID NO: 16. This non-limiting linker was designed to provide flexibility and solubility. However, other linkers may be suitable or even offer improved performance in the chimeric ligase polypeptide described herein.

Accordingly, in some embodiments, the chimeric ligase enzyme comprises a linker. The linker connects the ligase polypeptide sequence and the RNAseH polypeptide or the RNAseH domain fragment thereof. Any functional linker known in the art can be used. Any suitable linker, including without limitation any variation of G(n)S(m)G(p) linker can be inserted between the RT polypeptide and the DNA binding protein. In some embodiments, n=0, 1, 2, 3, 4, 5, 6, 7, 8, 9, 10 11, 12, 13, 14, 15, 16, 17, 18, 19 or 20, m=0, 1, 2, 3, 4, 5, 6, 7, 8, 9, 10 11, 12, 13, 14, 15, 16, 17, 18, 19 or 20, p=0, 1, 2, 3, 4, 5, 6, 7, 8, 9, 10 11, 12, 13, 14, 15, 16, 17, 18, 19 or 20, and n, m, and p are selected independently.

In some embodiments of the chimeric ligase polypeptide described herein, the linker comprises an amino acid sequence having at least about 90%, at least about 91%, at least about 92%, at least about 93%, at least about 94%, at least about 95%, at least about 96%, at least about 97%, at least about 98%, or at least about 99% sequence identity to SEQ ID NO: 17. In some embodiments, the linker comprises an amino acid sequence having at least about 90%, at least about 91%, at least about 92%, at least about 93%, at least about 94%, at least about 95%, at least about 96%, at least about 97%, at least about 98%, or at least about 99% sequence identity to SEQ ID NO: 18. In some embodiments, the linker comprises an amino acid sequence having at least about 90%, at least about 91%, at least about 92%, at least about 93%, at least about 94%, at least about 95%, at least about 96%, at least about 97%, at least about 98%, or at least about 99% sequence identity to SEQ ID NO: 19. In some embodiments, the linker comprises an amino acid sequence having at least about 90%, at least about 91%, at least about 92%, at least about 93%, at least about 94%, at least about 95%, at least about 96%, at least about 97%, at least about 98%, or at least about 99% sequence identity to SEQ ID NO: 20.

In some embodiments, the linker can comprise GGGGS or SEQ ID NO: 18. The linker can comprise GSGGSG or SEQ ID NO: 19. In some embodiment the linker can be encoded by SEQ ID NO: 20 or GGTTCAGGGGGTTCCGGT. The linker can comprise GGSGGS or SEQ ID NO: 17. The linker can be encoded by GGTGGCAGCGGTGGCAGC or SEQ ID NO: 16.

### F. Tags

The multidomain ligase enzymes (e.g., the chimeric ligase polypeptide) described herein can further comprise or one or more tags. The one or more tags can be used for any purposes including but not limited to purification and solubility. Exemplary tags can include, but not limited to an N-terminal histidine (N-His) tag, a C-terminal histidine (C-His) tag, or a C-His SET tag.

One aspect of the present disclosure provides a chimeric ligase polypeptide comprising: a ligase polypeptide domain; a catalytically inactive ribonuclease H (RNAseH) polypeptide domain; and a tag protein. The tag can be located at the N-terminus or C-terminus of the chimeric ligase polypeptide. The tag protein can be selected from the group consisting of an affinity tag, a fluorescent tag, or an expression and/or solubility enhancement tag.

In some embodiments, the tag protein is selected from hexahistidine tag (his-tag), Fasciola hepatica 8-kDa antigen tag (Fh8), Glutathione-S-transferase (GST) tag, maltose-binding protein tag (MBP), Flag tag peptide (FLAG tag), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin), small ubiquitin-like modifier tag (SUMO), a strep tag, Thioredoxin (Trx) tag, a VariFlex C-Terminal solubility enhancement tag, a short peptide C-terminal tag, Solubility-enhancer peptide sequences (SET) tag, IgG domain B1 of Protein G (GB1) tag, IgG repeat domain ZZ of Protein A (ZZ) tag, Mutated dehalogenase tag (HaloTag), Solubility eNhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Stress-responsive proteins tag (e.g., RpoA, tag, SlyD Tsf tag, RpoS tag, PotD tag, or Crr tag), and E. coli acidic proteins tag (e.g., msyB tag, yigD tag, and rpoD tag). Additional affinity tags and solubility enhancer tags are known to those skill in the art. See Costa et al., Front. Microbiol., 63(5): (2014); Esposito and Chatterjee Curr. Opin. Biotechnol., 17: 353-358 (2006); Malhotra, A. “Tagging for protein expression,” in Guide to Protein Purification, 2nd Edn, eds. R. R. Burgess and M. P. Deutscher (San Diego, CA: Elsevier), 463:239-258 (2009).

Tags used in the practice of the invention may serve any number of purposes and a number of tags may be added to impart one or more different functions to the engineered reverse transcriptase, and/or derivatives thereof, of the disclosure. For example, tags may (1) contribute to protein-protein interactions both internally within a protein and with other protein molecules, (2) make the protein amenable to particular purification methods, (3) enable one to identify whether the protein is present in a composition; or (4) give the protein other functional characteristics.

In one embodiment, the tag is an affinity tag selected from a histidine tag such as a hexahistidine tag (his-tag or 6 His-tag), Fasciola hepatica 8-kDa antigen tag (Fh8), Glutathione-S-transferase (GST) tag, maltose-binding protein tag (MBP), Flag tag peptide (FLAG), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin). In one embodiment, the tag is a hexahistidine tag.

In some embodiments, the tag is selected from a small ubiquitin-like modifier tag (SUMO), a VariFlex C-Terminal solubility enhancement tag, a short peptide C-terminal tag, Thioredoxin (Trx) tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), Flag tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

In some embodiments, In some embodiments, the solubility enhancer tag. In some embodiments, the solubility enhancer tag is selected from the group consisting of a SUMO tag, a GST tag, a Trx tag, a VariFlex C-Terminal solubility enhancement tag, a short peptide C-terminal tag, an Fh8 tag, MBP tag, SET tag, GB1 tag, ZZ tag, HaloTag, SNUT tag, Skp tag, T7PK tag, EspA tag, Mocr tag, Ecotin tag, CaBO tag, ArsC tag, IF2-domain I tag, Expressivity tag, RpoA, tag, SlyD, tag, Tsf tag, RpoS tag, PotD tag, Crr tag, msyB tag, yigD tag, and rpoD tag.

In some embodiments, the tag is an affinity tag. In one embodiment, the tag is an affinity tag and comprises a histidine purification tag. In one embodiment, the tag is a hexahistidine tag (his tag). In one embodiment, the tag comprises an amino acid sequence of the sequence HHHHHH (SEQ ID NO: 21). In one embodiment, the tag is a solubility enhancer tag. In one embodiment, the solubility enhancer tag is a short peptide C-terminal tag. In one embodiment, the solubility enhancer tag comprises an amino acid sequence of SEEDEEKEEDG (SEQ ID NO: 27) or an amino acid sequence having at least 90% sequence identity to the amino acid sequence of SEQ ID NO: 27.

In some embodiments, the tag further comprises an endoprotein cleavage site selected from ENLYFQ/G (SEQ ID NO: 22), DDDDK/(SEQ ID NO: 23), IEGR/(SEQ ID NO: 24), LVPR/GS (SEQ ID NO: 25), or LEVLFQ/GP (SEQ ID NO: 26).

In some embodiments, the chimeric ligase polypeptide further comprises a protease cleavage sequence. In some embodiments, the cleavage of the protease cleavage sequence by a protease results in cleavage of the affinity tag from the engineered reverse transcriptase polypeptide or recombinant RT protein or a derivative thereof. In some instances, the protease cleavage sequence/site is recognized by a protease including, but not limited to, alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase (EnTK), gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin (Thr), tissue kallikrein, tobacco etch virus (TEV), togavirin, tryptophanyl aminopeptidase, U-plasminogen activator, V8, venombin A, venombin AB, factor Xa (Xa), and Xaa-pro aminopeptidase. In some embodiments, the protease cleavage sequence is a thrombin cleavage sequence.

In some embodiments, the tag is cleaved or removed from the chimeric ligase polypeptide via the cleavage site. In one embodiment, the tag is cleaved or removed using an endoprotein selected from the group consisting of tobacco etch virus protease (Tev), enterokinase (EntK), factor Xa (Xa), thrombin (Thr), genetically engineered derivative of human rhinovirus 3C protease (PreScission), Catalytic core of Ulp1 (SUMO protease). In one embodiment, the tag is cleaved at ENLYFQ/G (SEQ ID NO: 22) using tobacco etch virus protease (Tev). In another embodiment, the tag is cleaved at DDDDK/(SEQ ID NO: 23) using Enterokinase (EntK). In another embodiment, the tag is cleaved at IEGR/(SEQ ID NO: 24) using Factor Xa (Xa). In another embodiment, the tag is cleaved at LVPR/GS (SEQ ID NO: 25) using thrombin (Thr). In another embodiment, the tag is cleaved at LEVLFQ/GP (SEQ ID NO: 26) using a genetically engineered derivative of human rhinovirus 3C protease. In another embodiment, the tag is cleaved with Catalytic core of Ulp1 (SUMO protease). Catalytic core of Ulp1 recognizes SUMO tertiary structure and cleaves at the C-terminal end of the conserved Gly-Gly sequence in SUMO.

In some embodiments, the chimeric ligase polypeptide comprises an affinity tag at the N-terminus or at the C-terminus of the amino acid sequence. In some embodiments, the affinity tag include, but is not limited to, albumin binding protein (ABP), AU1 epitope, AU5 epitope, T7-tag, V5-tag, B-tag, Chloramphenicol Acetyl Transferase (CAT), Dihydrofolate reductase (DHFR), AviTag, Calmodulin-tag, polyglutamate tag, E-tag, FLAG-tag, HA-tag, Myc-tag, NE-tag, S-tag, SBP-tag, Doftag 1, Softag 3, Spot-tag, tetracysteine (TC) tag, Ty tag, VSV-tag, Xpress tag, biotin carboxyl carrier protein (BCCP), green fluorescent protein tag, HaloTag, Nus-tag, thioredoxin-tag, Fc-tag, cellulose binding domain, chitin binding protein (CBP), choline-binding domain, galactose binding domain, maltose binding protein (MBP), Horseradish Peroxidase (HRP), Strep-tag, HSV epitope, Ketosteroid isomerase (KSI), KT3 epitope, LacZ, Luciferase, PDZ domain, PDZ ligand, Polyarginine (Arg-tag), Polyaspartate (Asp-tag), Polycysteine (Cys-tag), Polyphenylalanine (Phe-tag), Profinity eXact, Protein C, S1-tag, S1-tag, Staphylococcal protein A (Protein A), Staphylococcal protein G (Protein G), Small Ubiquitin-like Modifier (SUMO), Tandem Affinity Purification (TAP), TrpE, Ubiquitin, Universal, glutathione-S-transferase (GST), and poly(His) tag. In some instances, the affinity tag is at least 5 histidine amino acids.

In some embodiments, the chimeric ligase polypeptide may comprise an affinity tag at the N-terminus or at a C-terminus of the amino acid sequence. In some instances, the affinity tag may include, but is not limited to, albumin binding protein (ABP), AU1 epitope, AU5 epitope, T7-tag, V5-tag, B-tag, Chloramphenicol Acetyl Transferase (CAT), Dihydrofolate reductase (DHFR), AviTag, Calmodulin-tag, polyglutamate tag, E-tag, FLAG-tag, HA-tag, Myc-tag, NE-tag, S-tag, SBP-tag, Doftag 1, Softag 3, Spot-tag, tetracysteine (TC) tag, Ty tag, VSV-tag, Xpress tag, biotin carboxyl carrier protein (BCCP), green fluorescent protein tag, HaloTag, Nus-tag, thioredoxin-tag, Fc-tag, cellulose binding domain, chitin binding protein (CBP), choline-binding domain, galactose binding domain, maltose binding protein (MBP), Horseradish Peroxidase (HRP), Strep-tag, HSV epitope, Ketosteroid isomerase (KSI), KT3 epitope, LacZ, Luciferase, PDZ domain, PDZ ligand, Polyarginine (Arg-tag), Polyaspartate (Asp-tag), Polycysteine (Cys-tag), Polyphenylalanine (Phe-tag), Profinity eXact, Protein C, S1-tag, S1-tag, Staphylococcal protein A (Protein A), Staphylococcal protein G (Protein G), Small Ubiquitin-like Modifier (SUMO), Tandem Affinity Purification (TAP), TrpE, Ubiquitin, Universal, glutathione-S-transferase (GST), and poly(His) tag. In some instances, said affinity tag is at least 6 histidine amino acids (SEQ ID NO: 21).

In some embodiments, the chimeric ligase polypeptide described herein can comprise a protease cleavage sequence. In that embodiment, cleavage by a protease results in cleavage of the affinity tag from the engineered reverse transcription enzyme. In some instances, the protease cleavage sequence is recognized by a protease including, but not limited to, alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase. In some instances, the protease cleavage sequence is a thrombin cleavage sequence.

### III. Nucleic Acids and Expression Vectors

### A. Nucleic Acids

One aspect of the present disclosure provides an isolated nucleic acid molecule encoding the chimeric ligase polypeptide described herein. Another aspect of the present disclosure provides an isolated nucleic acid molecule encoding a chimeric ligase polypeptide comprising a ligase polypeptide domain; and a catalytically inactive ribonuclease H (RNAseH) polypeptide domain.

The chimeric ligase polypeptide described herein need not be encoded by any specific nucleic acid exemplified herein. For example, redundancy in the genetic code allows for variations in nucleotide codon sequences that nevertheless encode the same amino acid. Accordingly, chimeric ligase polypeptide of the present disclosure can be produced from nucleic acid sequences that are different from those set forth herein, for example, being codon optimized for a particular expression system. Codon optimization can be carried out, for example, as set forth in Athey et al., BMC Bioinformatics, 18:391-401 (2017).

Wild type nucleic acids (e.g., ligase polypeptide domain or RNAseH polypeptide domain) may be isolated from naturally occurring sources to be used as starting material to generate novel chimeric ligase. Generally, the nomenclature and the laboratory procedures in recombinant DNA technology described below are those well-known and commonly employed in the art. Standard techniques for cloning, DNA and RNA isolation, amplification and purification are known. Generally enzymatic reactions involving DNA ligase, DNA polymerase, restriction endonucleases are the like are performed according to the manufacturer's specifications. These techniques and various other techniques are generally performed according to Sambrook & Russell, Molecular Cloning—A Laboratory Manual, Cold Spring Harbor Laboratory, Cold Spring Harbor, N.Y., (1989) or Ausubel et al., Current Protocols in Molecular Biology, Vol. 1-3, John Wiley & Sons, Inc. (1994-1998).

The isolation of nucleic acids (e.g., ligase polypeptide domain or RNAseH polypeptide domain) may be accomplished by a variety of techniques. The nucleic acids of the present disclosure can be generated from the wild type sequences. The wild type sequences can be altered to create modified sequences. Wild type molecules (e.g., ligase polypeptide domain or RNAseH polypeptide domain) can be modified to create the chimeric ligase polypeptide described in the present application using methods that are well known in the art. Exemplary modification methods are site-directed mutagenesis, point mismatch repair, or oligonucleotide-directed mutagenesis.

Accordingly, in some embodiments, the isolated nucleic acid molecule comprises a nucleotide sequence that is at least 70%, at least 75%, at least 80%, at least 90% identical to SEQ ID NO: 28. In some embodiments, the isolated nucleic acid molecule comprises a nucleotide sequence of SEQ ID NO: 28. In some embodiments, the isolated nucleic acid molecule comprises a nucleotide sequence that is at least 70%, at least 75%, at least 80%, at least 90% identical to SEQ ID NO: 30. In some embodiments, the isolated nucleic acid molecule comprises a nucleotide sequence of SEQ ID NO: 30.

In some embodiments, the isolated nucleic acid molecule comprises a nucleotide sequence that is at least 70%, at least 75%, at least 80%, at least 90% identical to SEQ ID NO: 32. In some embodiments, the isolated nucleic acid molecule comprises a nucleotide sequence of SEQ ID NO: 32. In some embodiments, the isolated nucleic acid molecule comprises a nucleotide sequence that is at least 70%, at least 75%, at least 80%, at least 90% identical to SEQ ID NO: 34. In some embodiments, the isolated nucleic acid molecule comprises a nucleotide sequence of SEQ ID NO: 34.

In some embodiments, the isolated nucleic acid molecule comprises a nucleotide sequence that is at least 70%, at least 75%, at least 80%, at least 90% identical to SEQ ID NO: 35. In some embodiments, the isolated nucleic acid molecule comprises a nucleotide sequence of SEQ ID NO: 35. In some embodiments, the isolated nucleic acid molecule comprises a nucleotide sequence that is at least 70%, at least 75%, at least 80%, at least 90% identical to SEQ ID NO: 37. In some embodiments, the isolated nucleic acid molecule comprises a nucleotide sequence of SEQ ID NO: 37.

In some embodiments, the isolated nucleic acid molecule encoding the chimeric ligase polypeptide comprises a nucleotide sequence that is at least 70%, at least 75%, at least 80%, at least 90% identical to SEQ ID NO: 1. In some embodiments, the isolated nucleic acid molecule comprises a nucleotide sequence of SEQ ID NO: 1. In some embodiments, the isolated nucleic acid molecule comprises a nucleotide sequence that is at least 70%, at least 75%, at least 80%, at least 90% identical to SEQ ID NO: 3. In some embodiments, the isolated nucleic acid molecule comprises a nucleotide sequence of SEQ ID NO: 3. In some embodiments, the isolated nucleic acid molecule comprises a nucleotide sequence that is at least 70%, at least 75%, at least 80%, at least 90% identical to SEQ ID NO: 5. In some embodiments, the isolated nucleic acid molecule comprises a nucleotide sequence of SEQ ID NO: 5.

In some embodiments, the isolated nucleic acid molecule comprises a nucleotide sequence that is at least 70%, at least 75%, at least 80%, at least 90% identical to SEQ ID NO: 7. In some embodiments, the isolated nucleic acid molecule comprises a nucleotide sequence of SEQ ID NO: 7. In some embodiments, the isolated nucleic acid molecule comprises a nucleotide sequence that is at least 70%, at least 75%, at least 80%, at least 90% identical to SEQ ID NO: 9. In some embodiments, the isolated nucleic acid molecule comprises a nucleotide sequence of SEQ ID NO: 9. In some embodiments, the isolated nucleic acid molecule comprises a nucleotide sequence that is at least 70%, at least 75%, at least 80%, at least 90% identical to SEQ ID NO: 11. In some embodiments, the isolated nucleic acid molecule comprises a nucleotide sequence of SEQ ID NO: 11.

In some embodiments, the isolated nucleic acid molecule encodes an amino acid sequence of linker. In some embodiments, the isolated nucleic acid molecule encodes an amino acid sequence having at least about 90%, at least about 91%, at least about 92%, at least about 93%, at least about 94%, at least about 95%, at least about 96%, at least about 97%, at least about 98%, or at least about 99% sequence identity to any one of SEQ ID NO: 17, 18, 19, or 20.

In some embodiments, the isolated nucleic acid molecule encodes an amino acid sequence of the catalytically inactive RNAseH polypeptide domain. In some embodiments, the isolated nucleic acid molecule encodes an amino acid sequence having at least about 90%, at least about 91%, at least about 92%, at least about 93%, at least about 94%, at least about 95%, at least about 96%, at least about 97%, at least about 98%, or at least about 99% sequence identity to any one of SEQ ID NO: 13, 31, or 33. In some embodiments, the isolated nucleic acid molecule encodes n amino acid sequence set forth in SEQ ID NO: 13, 31, or 33. In some embodiments, the isolated nucleic acid molecule encodes the catalytically inactive RNAseH polypeptide domain. In some embodiments, the isolated nucleic acid molecule encodes a full-length E. coli RNAseH sequence or SEQ ID NO: 31. In some embodiments, the isolated nucleic acid molecule encodes a full-length S. tokodaii RNAseH sequence, or SEQ ID NO: 33.

In some embodiments, the isolated nucleic acid molecule encodes an amino acid sequence having at least 90%, at least 91%, at least 92%, at least 93%, at least 94%, at least 95%, at least 96%, at least 97%, at least 98%, or at least 99% identity to SEQ ID NO: 29, 36, 38, or 39. In some embodiments, the isolated nucleic acid molecule encodes an amino acid sequence having at least 90% identical to SEQ ID NO: 29.

### B. Vectors

Another aspect of the present disclosure comprises an expression vector comprising the isolated nucleic acid molecule described herein. encoding the chimeric ligase polypeptide described herein or derivatives thereof as described herein.

A “vector” refers to a polynucleotide, which when independent of the host chromosome, is capable replication in a host organism. Preferred vectors include plasmids and typically have an origin of replication. Vectors can comprise, e.g., transcription and translation terminators, transcription and translation initiation sequences, and promoters useful for regulation of the expression of the particular nucleic acid. The polymerases of the present disclosure can be expressed in a variety of host cells, including E. coli, other bacterial hosts, yeasts, filamentous fungi, and various higher eukaryotic cells such as the COS, CHO and HeLa cells lines and myeloma cell lines.

Techniques for gene expression in microorganisms are described in, for example, Smith, Gene Expression in Recombinant Microorganisms (Bioprocess Technology, Vol. 22), Marcel Dekker, 1994. Examples of bacteria that are useful for expression include, but are not limited to, Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus. Filamentous fungi that are useful as expression hosts include, for example, the following genera: Aspergillus, Trichoderma, Neurospora, Penicillium, Cephalosporium, Achlya, Podospora, Mucor, Cochliobolus, and Pyricularia. Synthesis of heterologous proteins in yeast is well known and described in the literature. There are many expression systems for producing the polymerase polypeptides of the present invention that are well known to those of ordinary skill in the art.

### C. Host Cells

Another aspect of the present disclosure provides a host cell transfected with the expression vector comprising the isolated nucleic acid molecule encoding the chimeric ligase polypeptide as described herein. Another aspect of the present disclosure provides a host cell transfected with the isolated nucleic acid molecule encoding the chimeric ligase polypeptide as described herein.

Eukaryotic expression systems for mammalian cells, yeast, and insect cells are well known in the art and are also commercially available. In yeast, vectors include Yeast Integrating plasmids (e.g., YIp5) and Yeast Replicating plasmids (the YRp series plasmids) and pGPD-2. Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Once expressed, the chimeric ligase polypeptide described herein or a derivative thereof can be purified according to standard procedures of the art, including ammonium sulfate precipitation, affinity purification columns, column chromatography, gel electrophoresis and the like. Substantially pure compositions of at least about 90 to about 95% homogeneity are preferred, and about 98 to about 99% or more homogeneity are most preferred. Once purified, partially or to homogeneity as desired, the polypeptides may then be used (e.g., as immunogens for antibody production).

To facilitate the purification of the chimeric ligase polypeptide or a derivative thereof described herein, the nucleic acids that encode the chimeric ligase polypeptide or derivatives thereof can also include a coding sequence for an epitope or “tag” for which an affinity binding reagent is available and described herein. Examples of suitable epitopes include the myc and V-5 reporter genes; expression vectors useful for recombinant production of fusion polypeptides having these epitopes are commercially available (e.g., Invitrogen (Carlsbad Calif.) vectors pcDNA3.1/Myc-His and pcDNA3.1/V5-His are suitable for expression in mammalian cells). Additional expression vectors suitable for attaching a tag to the fusion proteins of the disclosure, and corresponding detection systems are known to those of skill in the art as described herein, and several are commercially available (e.g., FLAG” (Kodak, Rochester N.Y.). Another example of a suitable tag is a polyhistidine sequence, which is capable of binding to metal chelate affinity ligands. Typically, six adjacent histidines are used (6 His-tag, his-tag). In some embodiments, the histidine tag comprises more than six histidines. In some embodiments, the histidine tag comprises less than six histidines. Suitable metal chelate affinity ligands that can serve as the binding moiety for a polyhistidine tag include nitrilo-tri-acetic acid (NTA).

One of skill in the art would recognize that after biological expression or purification, the chimeric ligase or derivatives thereof described herein may possess a conformation substantially different than the native conformations of the constituent polypeptides. In this case, it may be necessary or desirable to denature and reduce the chimeric ligase polypeptide or a derivative thereof and cause the chimeric ligase polypeptide or a derivative thereof to re-fold into the preferred conformation. Methods of reducing and denaturing proteins and inducing re-folding are well known to those of skill in the art.

### IV. Compositions and Reaction Mixtures

The present disclosure further provides compositions comprising a variety of components in various combinations needed RNA templated DNA ligation using any of the chimeric ligase polypeptides disclosed herein. One aspect of the present disclosure provides a composition comprising any of the chimeric ligase polypeptides described herein. Another aspect of the present disclosure provides a composition comprising any ligase polypeptide domains described herein. Another aspect of the present disclosure provides a composition comprising any of the catalytically inactive ribonuclease H (RNAseH) polypeptide domains described herein. Another aspect of the present disclosure provides a composition comprising any of the isolated nucleic acid molecules described herein. Another aspect of the present disclosure provides a composition comprising any of the host cells described herein.

In some embodiments, any one of the compositions described herein further comprise a ligation buffer. In some embodiments, the ligation buffer has a pH within a range of about 8 to about 8.5. In some embodiments, the ligation buffer has a pH of about 8.0 or about 8.5. In some embodiments, the buffer is any buffer disclosed in Table 2. In some embodiments, the buffer is selected from a control buffer, a Buffer 1A, a buffer 1B, or a buffer 2B.

In some embodiments of the present disclosure, the compositions are formulated by admixing one or more chimeric ligase polypeptide, or derivatives thereof of the present disclosure in a buffered salt solution. One or more DNA polymerases, one or more reverse transcriptases, one or more nucleotides, and/or one or more primers may optionally be added to create the compositions of the present disclosure. These compositions can be used in the methods disclosed herein to produce, analyze, quantitate and otherwise manipulate nucleic acid molecules (e.g., using reverse transcription or one-step RT-PCR procedures).

In some embodiments, the chimeric ligase polypeptide disclosed herein are provided at working concentrations (e.g., lx) in stable buffered salt solutions. The terms “stable” and “stability” as used herein generally mean the retention by a composition, such as an enzyme composition, of at least 70%, preferably at least 80%, and most preferably at least 90%, of the original enzymatic activity (in units) after the enzyme or composition containing the enzyme has been stored for about one week at a temperature of about 4° C., about two to six months at a temperature of about −20° C., and about six months or longer at a temperature of about −80° C.

As used herein, the term “working concentration” means the concentration of an enzyme that is at or near the optimal concentration used in a solution to perform a particular function such as reverse transcription of nucleic acids.

Such compositions can also be formulated as concentrated stock solutions (e.g., 2×, 3×, 4×, 5×, 6×, 10×, etc.). In some embodiments, having the composition as a concentrated (e.g., 5×) stock solution allows a greater amount of nucleic acid sample to be added (such as, for example, when the compositions are used for nucleic acid synthesis). The water used in forming the compositions of the present invention is preferably distilled, deionized and sterile filtered (through a 0.1-0.2 micrometer filter) and is free of contamination by DNase and RNase enzymes. Such water is available commercially, for example from Life Technologies (Carlsbad, Calif.) or may be made as needed according to methods well known to those skilled in the art.

### V. Methods of Using Chimeric Ligase Polypeptide

The chimeric ligase polypeptide of the present disclosure can be used as a ligase for any reaction involving the ligation of two nucleic acid ends. Nonlimiting applications of use include practicing templated ligation methods for sequencing assays, including single cell sequencing such as single cell RNA-seq (scRNA-seq) technology, spatial sequencing assays such as spatial transcriptomic analysis, and in situ sequencing methods where templated ligation methods are practiced. In some embodiments, the templated ligation method is RNA-templated DNA ligation.

One aspect of the present disclosure provides a method for ligating single-stranded DNA, the method comprising contacting under suitable conditions a sample comprising a nucleic acid template and DNA probes with a composition comprising any of the chimeric ligase polypeptides described herein. In some embodiments, the composition comprises a s ligation buffer. In some embodiments, the ligation buffer is control Buffer, Buffer 1A, Buffer 1B, Buffer 2B, Buffer 3A, or any equivalent buffer thereof. In some embodiments, the suitable ligation buffer has a pH in the range of about 7 to about 9. In some embodiments, the suitable ligation buffer has a pH in the range of about 8 to about 8.5. In some embodiments, the suitable ligation buffer has a pH of at least about 8.0, 8.1, 8.2, 8.3, 8.4, or 8.5. In some embodiments, the sample comprises a cell, a cell bead, a permeabilized cell, a nucleus, a tissue, or a cellular preparation.

In some embodiments, the sample comprises a nucleus and the nucleus is optionally permeabilized. In some embodiments, the sample comprises cells in suspension, fresh cells, or fixed cells. In some embodiments, the sample comprises cells and tissues immobilized on various solid surfaces. In some embodiments, the nucleic acid template is an RNA, a DNA, or a nucleic acid comprising an unnatural nucleotide. In some embodiments, the nucleic acid template comprises RNA.

The chimeric ligase polypeptide of the present disclosure is suitable for use as a replacement ligase in all applications for which PBCV-1 ligase and other ligases (e.g., T4 ligase) are used. Certain uses of the PBCV-1 ligase are found in, for example, U.S. Pat. Nos. 10,597,650 and 10,597,710. In certain embodiments, the chimeric ligase polypeptide of the present disclosure is suitable for use in efficiently ligating DNA probes that are adjacently hybridized to RNA templates.

In some aspects, the present disclosure provides a method for processing RNA in a biological sample, the method including (a) contacting the biological sample with one or more DNA oligonucleotides that include a 3′ DNA hybridization region and a 5′ DNA hybridization region, the hybridization regions being complementary to target sequences in the RNA, wherein the contacting of (a) is performed under a set of conditions to allow the hybridization regions to anneal to the target sequences, and (b) contacting the biological sample with a chimeric ligase as described herein, wherein the contacting in (b) is performed under a set of conditions to allow for ligation of the 3′ DNA hybridization region and the 5′ DNA hybridization region.

Another aspect of the present disclosure provides a method for detecting RNA sequences in a biological sample, the method comprising contacting the biological sample with at least one DNA probe comprising a 3′ DNA hybridization region and a 5′ DNA hybridization region that anneal to a first and a second complementary target sequences in the target RNA sequence in the biological sample; ligating the 3′ DNA hybridization region and the 5′ DNA hybridization region using any of the chimeric ligase polypeptide described herein to generate a ligation product; amplifying the ligation product; and detecting the amplification product, thereby detecting the RNA sequence.

In some embodiments, the 5′ hybridization region is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides in length. In some embodiments, the 3′ hybridization region is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides in length.

### A. Probes

In some aspects, provided herein are methods that involve ligation of one or more nucleic acid probes. In some embodiments, the one or more nucleic acid probes are DNA probes. In some embodiments, the DNA probe or a DNA probe set is capable of RNA-templated DNA ligation.

In some embodiments, the DNA probe is a circularizable DNA probe. A suitable circularizable probe or probe set may be a circularizable rolling circle amplification (RCA) template in the form of a linear molecule having ligatable ends (e.g., end that can be ligated), which may circularize by ligating the ends together directly or indirectly. A circularizable template may also be provided in two or more parts, namely two or more oligonucleotides, which may be ligated together to form a circle. In some embodiments, the RCA template is circularized by ligation prior to RCA.

In some embodiments, the one or more nucleic acid probes comprises two nucleic acid probes. In some embodiments, the two nucleic acid probes comprise a first DNA probe comprising a 3′ hybridization region, and a second DNA probe comprising a 5′ hybridization region.

In some embodiments, the circularizable probe comprises one or more barcodes. In some embodiments, one or more barcodes are indicative of a sequence in the target nucleic acid, such as a single nucleotide of interest (e.g., SNPs or point mutations), a dinucleotide sequence, or a short sequence of about 5 nucleotides in length.

The ligation described herein can be a direct or indirect ligation. In some embodiments, ligatable probe ends are directly ligated. As used herein, a direct ligation can refer to ligation of the ends of the DNA probe hybridize immediately adjacently to one another in the target RNA template to form a substrate for a ligase enzyme resulting in their ligation to each other (intramolecular ligation). In some embodiments, the 3′ DNA hybridization region and the 5′ DNA hybridization anneal to the first and the second complementary target sequences and the first and the second complementary target sequences are adjacent in the target RNA sequence.

Alternatively, in some embodiments, ligateable probe ends are indirectly ligated. As sued herein indirect ligation can refer to ligation of the ends of the DNA probe hybridize non-adjacently to one another. For example, the 3′ DNA hybridization region and the 5′ DNA hybridization region are separated by one or more intervening nucleotides or “gap.” In some embodiments, the 3′ DNA hybridization region and the 5′ DNA hybridization anneal to the first and the second complementary target sequences and the first and second complementary target sequences are separated by one or more intervening nucleotides. In some embodiments, the first and second complementary target sequences are separated by a gap of about 1 to 40 nucleotides; about 1 to 60 nucleotides; or about 3 to 40 nucleotides.

In some embodiments, the gap is about 1, about 2, about 3, about 4, about 5, about 6, about 7, about 8, about 9, about 10, about 15, about 20 or more nucleotides. In some embodiments of the method for detecting RNA sequences in a biological sample described herein, the 3′ DNA hybridization region and the 5′ DNA hybridization region of the DNA probe are ligated without gap filling. In some embodiments of the method for detecting RNA sequences in a biological sample described herein, the method further comprises filling the gap prior to ligating the 3′ DNA hybridization region and the 5′ DNA hybridization region.

In some embodiments, the gap is filled by extension of the 3′ DNA hybridization region of the DNA probe. In some embodiments, the gap is filled by one or more gap oligonucleotides which are complementary to the DNA probe, or the target RNA. In some embodiments, ligation comprises ligating the 3′ DNA hybridization region and/or the 5′ DNA hybridization region the probe to at least one gap oligonucleotide, such that the gap oligonucleotide becomes incorporated into the resulting polynucleotide. The gap oligonucleotide is a splint or a splint primer. In one embodiment, the splint primer is complementary to the 3′ DNA hybridization region or the 5′ DNA hybridization region of the DNA probe. In another embodiment, the splint primer is complementary to the 3′ DNA hybridization region and the 5′ DNA hybridization region of the DNA probe.

Another aspect of the present disclosure provides a method for identifying the location of a nucleic acid analyte in a biological sample, the method comprising: (a) contacting the biological sample with a first substrate; (b) hybridizing a first probe and a second probe to the nucleic acid analyte; (c) ligating the first probe and the second probe with any chimeric ligase polypeptides described herein, thereby creating a ligation product; (d) releasing: (i) the ligation product from the nucleic acid analyte, and (ii) the blocking probe from the capture binding domain; (e) hybridizing the capture binding domain to the capture domain; and (f) determining (i) all or part of the sequence of the ligation product, or a complement thereof, and (ii) the sequence of the spatial barcode, or a complement thereof, and using the determined sequences of (i) and (ii) to identify a location of the nucleic acid analyte in the biological sample.

In some embodiments, the second probe comprises a capture binding domain that hybridizes to a capture domain of a capture probe. In some embodiments, the capture probe is affixed to the first substrate or the capture probe is affixed to a second substrate. In some embodiments, the capture probe further comprises a spatial barcode. In some embodiments, a portion of the capture binding domain is hybridized to a blocking probe. In some embodiments, the ligation product comprises a sequence that is substantially complementary to the nucleic acid analyte.

### B. Ligation

During ligation, the reaction mixture in certain embodiments may be maintained at a temperature ranging from about 4° C. to about 105° C., about 4 to about 80° C., such as about 10 to about 70° C., about 15 to about 60° C., typically such as from about 20° C. to about 37° C. for a period of time ranging from about 5 seconds to about 16 hours, such as from about 1 minute to about 1 hour. In yet other embodiments, the reaction mixture may be maintained at a temperature ranging from about 35° C. to about 45° C., such as from about 37° C. to about 42° C., e.g., at or about 38° C., 39° C., 40° C. or 41° C., for a period of time ranging from about 5 seconds to about 16 hours, such as from about 1 minute to about 1 hour, including from about 2 minutes to about 8 hours. In a representative embodiment, the ligation reaction mixture includes 50 mM Tris pH7.5, 10 mM MgCI2, 10 mM DTT, 1 mM ATP, 25 mg/ml BSA, 0.25 units/ml RNase inhibitor, and a chimeric ligase polypeptide described herein at 0.125 units/ml. In yet another representative embodiment, 2.125 mM magnesium ion, 0.2 units/ml RNase inhibitor; and 0.125 units/ml a chimeric ligase polypeptide described herein can be employed.

It will be evident to a person of skill in the art that the ligation conditions may depend on the ligase enzyme used in the methods disclosed herein. Hence, the above-described ligation conditions are merely a representative example and the parameters may be varied according to well-known protocols. However, it will further be understood that the alteration of one parameter (e.g., increasing the temperature), may require the modification of other conditions to ensure that other steps of the assay are not inhibited or disrupted (e.g., binding of the probe to the target nucleic acid molecule). Such manipulation of ligase assay methods is routine in the art.

### C. Amplification

In some embodiments, once the ligated probe is formed, it is amplified in order to increase the copy number of the ligation product in a sample, which may increase the sensitivity of the detection method of the present invention. In certain embodiments, a part or portion of the ligated probe is amplified. The ligated probe (or part thereof) may be amplified using any convenient method known in the art, such as PCR or a variant thereof, such as rolling circle amplification (RCA), SDA, HAD, LAMP, or SMAP.

In some embodiments, the probe is circularized by ligation. Preferably in such embodiments, the probe may be amplified by rolling circle amplification (RCA). Rolling circle amplification using a circular template for amplification provides a concatenated RCA product comprising multiple tandem repeats, each having a sequence complementary to the circular template oligonucleotide (i.e., the circularized ligation product). RCA may be performed, for example, by contacting the sample with a separate primer complementary to and capable of hybridizing to the resulting circle, and the 3′ end of said primer may be extended to provide a concatenated RCA product. Alternatively, the target nucleic acid molecule may comprise or provide a 3′ end which can serve as a primer for RCA amplification.

In some embodiments, amplification of the ligated probe is performed using a DNA polymerase enzyme, i.e., a polymerase enzyme capable of synthesizing a DNA oligonucleotide from dNTPs. The term “DNA polymerase” as used herein includes any enzyme capable of incorporating dNTPs, and hence includes enzymes which have both DNA and RNA polymerase activity. In particular, the DNA polymerase may be an enzyme whose principle activity or function is to synthesize DNA, but which is also capable of incorporating RNA nucleotides. A DNA polymerase may be selected which is capable of using a chimeric DNA-RNA oligonucleotide as a template for amplification, as the ligated probe comprises at least one ribonucleotide.

### VI. Sequencing Assay Methods

In some aspects, provided herein are ligation-based sequencing assays using a chimeric ligase as disclosed herein. Non-limiting aspects of ligation-based sequencing assays are described in US20210040551A1, which is incorporated herein by reference in its entirety. In some aspects, the sequencing assays provided herein include: (a) contacting a biological sample with at least one DNA probe, wherein the at least one DNA probe comprise a 3′ DNA hybridization region and a 5′ DNA hybridization region that anneal to a first and a second complementary target sequences in a target RNA sequence in the biological sample; (b) ligating the 3′ DNA hybridization region and the 5′ DNA hybridization region using a chimeric ligase polypeptide as provided herein to generate a ligation product; and (c) detecting the ligation product, or an extension product or amplicon thereof, thereby detecting the RNA sequence. In some aspects, the sequencing assays is selected from: a single cell sequencing assay (e.g., including single cell barcodes), a spatial analysis assay (e.g., involving spatial barcodes), or in situ sequencing assay (e.g., directly sequencing templates present in a cell or tissue sample).

In some embodiments, the sequencing assay is a spatial analysis assay. In some embodiments, the spatial analysis assay includes: contacting a biological sample with at least one DNA probe, wherein the at least one DNA probe comprise a 3′ DNA hybridization region and a 5′ DNA hybridization region that anneal to a first and a second complementary target sequences in a target RNA sequence in the biological sample; ligating the 3′ DNA hybridization region and the 5′ DNA hybridization region using a chimeric ligase polypeptide as provided herein to generate a ligation product; and detecting the ligation product, or an extension product or amplicon thereof, thereby detecting the RNA sequence, wherein the biological sample comprises cells or tissue immobilized on a surface. In some embodiments, the spatial analysis assay further comprises, after the ligating: contacting the biological sample with barcode nucleic acid molecules comprising spatial barcodes, hybridizing the ligated product to the barcode nucleic acid molecules, and extending the ligation product to generate extended ligation products comprising the spatial barcodes, wherein the ligated product comprises a sequence that hybridizes to the barcode nucleic acid molecules. In some embodiments, the spatial assay further includes isolating the extended ligation product, and sequencing the isolated, extended ligation product, or amplicons thereof.

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev F, dated January 2022); and/or the Visium Spatial Gene Expression Reagent Kits—Tissue Optimization User Guide (e.g., Rev E, dated February 2022), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of PCT Publication No. WO2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

In some aspects, the sequencing assay is a single cell sequencing assay. In some aspects, the single cell sequencing assay includes: contacting a biological sample including cells, nuclei, or cell beads with at least one DNA probe, wherein the at least one DNA probe comprise a 3′ DNA hybridization region and a 5′ DNA hybridization region that anneal to a first and a second complementary target sequences in a target RNA sequence in the biological sample; partitioning single cells, single nuclei, or single cell beads of the biological sample into discrete partitions; ligating the 3′ DNA hybridization region and the 5′ DNA hybridization region using a chimeric ligase polypeptide as provided herein to generate a ligation product; and detecting the ligation product, or an extension product or amplicon thereof, thereby detecting the RNA sequence. In some embodiments, the discrete partitions each comprise a nucleic acid molecule comprising a barcode nucleic acid molecule comprising a partition-specific barcode, and the method further comprises, after the ligating: contacting the biological sample with barcode nucleic acid molecules comprising partition-specific barcodes, hybridizing the ligated product to the barcode nucleic acid molecules, and extending the ligation product to generate extended ligation products comprising the partition-specific barcodes, wherein the ligated product comprises a sequence that hybridizes to the barcode nucleic acid molecules. In some aspects, the single cell assay further comprises amplifying the extended ligation product prior to sequencing. In some aspects, the single cell assay further comprises isolating the extended ligation product from the partitions prior to sequencing. Non-limiting aspects of single cell sequencing assays are described in U.S. Ser. No. 11/655,499B1; which is incorporated herein by reference in its entirety.

In some aspects, the sequencing assay is an in situ sequencing assay. In some embodiments, the in situ assay includes: contacting a biological sample with at least one DNA probe, wherein the at least one DNA probe comprise a 3′ DNA hybridization region and a 5′ DNA hybridization region that anneal to a first and a second complementary target sequences in a target RNA sequence in the biological sample; ligating the 3′ DNA hybridization region and the 5′ DNA hybridization region using a chimeric ligase polypeptide as provided herein to generate a ligation product; and in situ detecting the ligation product, or an extension product or amplicon thereof, thereby detecting the RNA sequence. In some embodiments, the sample comprises cells or tissue immobilized on a surface. In some embodiments, the method further comprises amplifying the ligation product, and the detecting comprises in situ detecting the amplified ligation product. In some embodiments, the at least one DNA probe is a circularizable probe, and the amplifying comprises rolling circle amplification. In some embodiments, the at least one DNA probe comprises a barcode, optionally wherein the barcode comprises a UMI. In some embodiments, the in situ detecting comprises fluorescent probe hybridization and imaging. Non-limiting aspects of in situ sequencing assays are described in US20210262018A1; which is incorporated herein by reference in its entirety.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Publication No. WO2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples—which can be from different tissues or organisms—assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these into a single recipient (microarray) block at defined array coordinates.

The biological sample as used herein can be any suitable biological sample described herein or known in the art. In some embodiments, the biological sample is a tissue. In some embodiments, the tissue sample is a solid tissue sample. In some embodiments, the biological sample is a tissue section. In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed using cryosectioning. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plants, an insect, an arachnid, a nematode (e.g., Caenorhabditis elegans), a fungi, an amphibian, or a fish (e.g., zebrafish)). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example methanol. In some embodiments, instead of methanol, acetone, or an acetone-methanol mixture can be used. In some embodiments, the fixation is performed after sectioning. In some instances, the biological sample is not fixed with paraformaldehyde (PFA). In some instances, when the biological sample is fixed with a fixative including an alcohol (e.g., methanol or acetone-methanol mixture), it is not decrosslinked afterward. In some preferred embodiments, the biological sample is fixed with a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen.” In some embodiments, fixation of the biological sample e.g., using acetone and/or alcohol (e.g., methanol, acetone-methanol) is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing e.g., by formalin or PFA, the biological sample is dehydrated via sucrose gradient. In some instances, the fixed biological sample is treated with a sucrose gradient and then embedded in a matrix e.g., OCT compound. In some instances, the fixed biological sample is not treated with a sucrose gradient, but rather is embedded in a matrix e.g., OCT compound after fixation. In some embodiments when a fixed frozen tissue sample is treated with a sucrose gradient, it can be rehydrated with an ethanol gradient. In some embodiments, the PFA or formalin fixed biological sample, which can be optionally dehydrated via sucrose gradient and/or embedded in OCT compound, is then frozen e.g., for storage or shipment. In such instances, the biological sample is referred to as “fixed frozen.” In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated in an ethanol gradient.

In some instances, the biological sample (e.g., a fixed frozen tissue sample) is treated with a citrate buffer. Citrate buffer can be used for antigen retrieval to decrosslink antigens and fixation medium in the biological sample. Thus, any suitable decrosslinking agent can be used in addition to or alternatively to citrate buffer. In some embodiments, for example, the biological sample (e.g., a fixed frozen tissue sample) is decrosslinked with TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, it is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, it can be rehydrated with an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, acid and a soluble organic compound that preserves morphology and bio-molecules. It is a two-reagent fixative system in which tissue is firstly fixed in a solution containing methanol and acetic acid then stabilized in a solution containing ethanol. See, Ergin B. et al., J Proteome Res. 9(10):5188-96 (2010); Kap M. et al., PLoS One. 6(11):e27704 (2011); and Mathieson W. et al., Am J Clin Pathol. 146(1):25-40 (2016), each of which are hereby incorporated by reference in their entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample is fixed, for example in methanol, acetone, acetone-methanol, PFA, PAXgene or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RTL methods disclosed herein. A limitation of direct RNA capture for fixed samples is that the RNA integrity of fixed (e.g., FFPE) samples can be lower than a fresh sample, thereby making it more difficult to capture RNA directly, e.g., by capture of a common sequence such as a poly(A) tail of an mRNA molecule. However, by utilizing RTL probes that hybridize to RNA target sequences in the transcriptome, one can avoid a requirement for RNA analytes to have both a poly(A) tail and target sequences intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Non-limiting examples of stains include histological stains (e.g., hematoxylin and/or eosin) and immunological stains (e.g., fluorescent stains). The biological sample can be stained using Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. In some instances, PAS staining is performed after formalin or acetone fixation. In some embodiments, a biological sample (e.g., a fixed and/or stained biological sample) can be imaged. Biological samples are also described in Section (I)(d) of PCT Publication No. WO2020/176788 and/or U.S. Patent Application Publication No. 2020/0277663.

The following embodiments can be used with any of the methods described herein. In some embodiments, the biological sample is imaged. In some embodiments, the biological sample is visualized or imaged using bright field microscopy. In some embodiments, the biological sample is visualized or imaged using fluorescence microscopy. Additional methods of visualization and imaging are known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding the primer to the biological sample.

In some embodiments, the method includes staining the biological sample. In some embodiments, the staining includes the use of hematoxylin and eosin. In some embodiments, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label selected from the group consisting of a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Publication No. WO2020/176788 and/or U.S. Patent Application Publication No. 2020/0277663. Briefly, in any of the methods described herein, the method includes a step of permeabilizing the biological sample. For example, the biological sample can be permeabilized to facilitate transfer of the extension products to the capture probes on the array. In some embodiments, the permeabilizing includes the use of an organic solvent (e.g., acetone, ethanol, and methanol), a detergent (e.g., saponin, Triton X-100™, Tween-20™, or sodium dodecyl sulfate (SDS)), an enzyme (an endopeptidase, an exopeptidase, a protease), or combinations thereof. In some embodiments, the permeabilizing includes the use of an endopeptidase, a protease, SDS, polyethylene glycol tert-octylphenyl ether, polysorbate 80, and polysorbate 20, N-lauroylsarcosine sodium salt solution, saponin, Triton X-100™, Tween-20™, or combinations thereof. In some embodiments, the endopeptidase is pepsin. In some embodiments, the endopeptidase is Proteinase K. Additional methods for sample permeabilization are described, for example, in Jamur et al., Method Mol. Biol. 588:63-66(2010), the entire contents of which are incorporated herein by reference.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663. Generation of capture probes can be achieved by any appropriate method, including those described in Section (II)(d)(ii) of PCT Publication No. WO2020/176788 and/or U.S. Patent Application Publication No. 2020/0277663.

In some embodiments, the biological sample is mounted on a first substrate and the substrate comprising the array of capture probes is a second substrate. During this process, one or more analytes or analyte derivatives (e.g., intermediate agents; e.g., ligation products) are released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described e.g., in U.S. Patent Application Pub. No. 2021/0189475 and PCT Pub. Nos. WO 2021/252747 A1, WO 2022/061152 A2, and WO 2022/140028 A1.

### VII. Kits

One aspect of the present invention provides a kit comprising the chimeric ligase polypeptide or a derivative thereof as described herein. In some embodiments, the kit further comprises one or more of a vector, a nucleotide, a buffer, a salt, and/or instructions. In another embodiment, a kit may comprise a chimeric ligase polypeptide or a derivative thereof for use in RNA-templated DNA ligation assay. In yet another embodiment, a kit may be used for single cell profiling of the transcriptome. In yet another embodiment, a kit may be used for spatial transcriptomics methods and assays. In yet another embodiment, a kit may be used for in situ methods and assays.

The kit may include suitable reaction buffers, dNTPs, one or more primers, one or more control reagents, or any other reagents disclosed for performing the methods of the present disclosure. The chimeric ligase polypeptide or a derivative thereof, reaction buffer, and dNTPs may be provided separately or may be provided together in a master mix solution. When the chimeric ligase polypeptide or a derivative thereof, reaction buffer, and dNTPs are provided in a master mix, the master mix is present at a concentration at least two times the working concentration indicated in instructions for use in an extension reaction. In other cases, the master mix may be present at a concentration at least three times, at least four times, at least five times, at least six times, at least seven times, at least eight times, at least nine times, or at least ten times, the working concentration indicated. The primer in the kits may be a poly-dT primer, a random N-mer primer, or a target-specific primer.

The kits may further include one, two, three, four, five or more, up to all of partitioning fluids, including both aqueous buffers and non-aqueous partitioning fluids or oils, nucleic acid barcode capture probes that are releasably associated with beads, as described herein, microfluidic devices, reagents for disrupting cells, reagents for amplifying nucleic acids, as well as instructions for using any of the foregoing in the methods described herein.

The instructions for using any of the methods are generally recorded on a suitable recording medium (e.g., printed on a substrate such as paper or plastic), or available in a digital format. As such, the instructions may be present in the kits as a package insert, in the labeling of the container of the kit or components thereof (i.e., associated with the packaging or subpackaging). In some cases, the instructions may be present as an electronic storage data file present on a suitable computer readable storage medium. In other cases, the actual instructions may not be present in the kit but means for obtaining the instructions from a remote source (e.g., via the internet) may be provided). For example, a kit that includes a web address where the instructions may be viewed and/or from which the instructions may be downloaded. As with the instructions, this means for obtaining the instructions is recorded on a suitable substrate.

Kits according to this aspect of the present disclosure comprise a carrier means, such as a box, carton, tube or the like, having in close confinement therein one or more container means, such as vials, tubes, ampoules, bottles and the like. In some embodiments, a first container means contains one or more of the chimeric ligase polypeptide or derivatives thereof of the present disclosure having ligase activity in RNA templated DNA ligation assay or similar ligation assay. When more than one polypeptide having the ligase activity is used, they may be in a single container as mixtures of two or more chimeric ligase polypeptides or derivatives thereof, or in separate containers. The kits of the disclosure can also comprise (in the same or separate containers) one or more DNA polymerases, one or more DNA reverse transcriptase, a suitable buffer, one or more nucleotides and/or one or more primers.

The kits of the disclosure can also comprise one or more hosts or cells including those that are competent to take up nucleic acids (e.g., DNA molecules including vectors). Preferred hosts may include chemically competent or electrocompetent bacteria such as E. coli (including DH5, DH5a, DH10B, HB101, Top 10, and other K-12 strains as well as E. coli B and E. coli W strains).

In a specific aspect of the disclosure, the kits of the disclosure (e.g., reverse transcription and amplification kits) can include one or more components (in mixtures or separately) including one or more chimeric ligase polypeptide or derivative thereof having ligase activity of the disclosure, one or more nucleotides (one or more of which may be labeled, e.g., fluorescently labeled) used for synthesis of a nucleic acid molecule, and/or one or more primers (e.g., oligo(dT) for reverse transcription, randomers for extension reactions, etc.). Such kits can further comprise one or more DNA polymerases.

### VIII. Definitions

Unless defined otherwise, all technical and scientific terms used herein generally have the same meaning as commonly understood by one of ordinary skill in the art to which this technology belongs. As used in this specification and the appended claims, the singular forms “a,” “an” and “the” include plural referents unless the content clearly dictates otherwise. “A and/or B” is used herein to include all of the following alternatives: “A,” “B,” “A or B,” and “A and B.” For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

By “analyte” is intended a biological molecule. Analytes include but are not limited to a DNA analyte, an RNA analyte, an oligonucleotide, a reporter molecule, a reporter molecule configured to directly couple to a protein, a reporter molecule configured to indirectly couple to a protein, a reporter molecule configured to directly couple to a metabolite, and a reporter molecule configured to indirectly couple to a metabolite.

The terms “adaptor(s),” “adapter(s)” and “tag(s)” may be used synonymously. An adaptor or tag can be coupled to a polynucleotide sequence to be “tagged” by any approach, including ligation, hybridization, or other approaches.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “barcoded nucleic acid molecule” generally refers to a nucleic acid molecule that results from, for example, the processing of a nucleic acid barcoded molecule with a nucleic acid sequence (e.g., nucleic acid sequence complementary to a nucleic acid primer sequence encompassed by the nucleic acid barcoded molecule). The nucleic acid sequence may be a targeted sequence or a non-targeted sequence. The nucleic acid barcoded molecule may be coupled to or attached to the nucleic acid molecule comprising the nucleic acid sequence. For example, a nucleic acid barcoded molecule described herein may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcoded molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcoded molecule to generate the barcoded nucleic acid molecule, or the nucleic acid molecule comprising the nucleic acid sequence may be attached to the nucleic acid barcoded molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

A nucleic acid barcoded molecule of a plurality of nucleic acid molecules may be used to generate a “barcoded nucleic acid molecule.” In some cases, a barcoded molecule comprises a different reporter barcode sequence that identifies a second analyte. A different reporter barcode sequence or an analyte-specific barcode sequence may identify a protein, a lipid, a metabolite or other second analyte.

By “binding sequence” is intended a nucleic acid sequence capable of binding to an analyte.

As used herein, a “capture probe capture domain” is a sequence, domain, or moiety that can bind specifically to a capture domain of a capture probe. In some embodiments, “capture domain capture domain” can be used interchangeably with “capture probe binding domain.” In some embodiments, a second probe includes a sequence from 5′ to 3′: a sequence that is substantially complementary to a sequence in the analyte and a capture probe capture domain.

In some embodiments, a capture probe capture domain includes a poly(A) sequence. In some embodiments, the capture probe capture domain includes a poly-uridine sequence, a poly-thymidine sequence, or both. In some embodiments, the capture probe capture domain includes a random sequence (e.g., a random hexamer or octamer). In some embodiments, the capture probe capture domain is complementary to a capture domain in a capture probe that detects a particular target(s) of interest. In some embodiments, a capture probe capture domain blocking moiety that interacts with the capture probe capture domain is provided. In some embodiments, a capture probe capture domain blocking moiety includes a sequence that is complementary or substantially complementary to a capture probe capture domain. In some embodiments, a capture probe capture domain blocking moiety prevents the capture probe capture domain from binding the capture probe when present. In some embodiments, a capture probe capture domain blocking moiety is removed prior to binding the capture probe capture domain (e.g., present in a ligated probe) to a capture probe. In some embodiments, a capture probe capture domain blocking moiety includes a poly-uridine sequence, a poly-thymidine sequence, or both. In some embodiments, the capture probe capture domain sequence includes ribonucleotides, deoxyribonucleotides, and/or synthetic nucleotides that are capable of participating in Watson-Crick type or analogous base pair interactions. In some embodiments, the capture probe binding domain sequence includes at least 10, 11, 12, 13, 14, 15, 16, 17, 18, 19, or 20 nucleotides. In some embodiments, the capture probe binding domain sequence includes at least 25, 30, or 35 nucleotides.

As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

The term “DNA polymerase” includes not only naturally-occurring enzymes but also all modified derivatives thereof, also including derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g., DNA polymerase activity of the wild-type sequence. Mutations can affect the activity profile of the enzymes, e.g., enhance or reduce the rate of polymerization, under different reaction conditions, e.g., temperature, template concentration, primer concentration, etc. Mutations or sequence-modifications can also affect the exonuclease activity and/or thermostability of the enzyme.

The term “derived from” encompasses the terms “originated from,” “obtained from,” “obtainable from,” “isolated from,” and “created from,” and generally indicates that one specified material finds its origin in another specified material or has features that can be described with reference to another specified material.

The terms “detectable label,” “optical label,” and “label” are used interchangeably herein to refer to a directly or indirectly detectable moiety that is associated with (e.g., conjugated to or bound to) a molecule to be detected, e.g., a capture probe or analyte, or a molecule that binds to a capture probe or analyte. The detectable label can be directly detectable by itself (e.g., radioisotope labels, fluorescent labels, or metal labels) or, in the case of an enzymatic label, can be indirectly detectable, e.g., by catalyzing chemical alterations of a chemical substrate compound or composition, which chemical substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, dyes, and metals.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a capture probe associated with a feature. For example, detectably labeled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to or bound to a feature, capture probe, or composition to be detected. For example, detectable labels can be incorporated during nucleic acid polymerization or amplification (e.g., Cy5®-labelled nucleotides, such as Cy5®-dCTP). Detectable labels can be associated with an oligonucleotide or peptide that is complementary or will associate with a nucleic acid or protein inside or on a cell.

Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7® Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM@1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™ Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorophyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Re®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rhol01, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

In some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate chemical substrates (e.g., an oxidizing reagent plus a chemiluminescent compound). A number of compound families are known to provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and—methoxy substituents, oxalates such as oxalyl active esters, p-nitrophenyl, N-alkyl acridinium esters, luciferins, lucigenins, or acridinium esters.

The term “duplex” herein refers to a region of complementarity that exists between two polynucleotide sequences. The term “duplex region” refers to the region of sequence complementarity that exists between two oligonucleotides or two portions of a single oligonucleotide. A duplex is a double stranded nucleic acid and may comprise two strands having the same or different length. The duplex may be a fully or partially double stranded nucleic acid duplex. The duplex may be a DNA:DNA duplex, or a DNA:RNA duplex.

As used herein, the term “efficiency” in the context of a nucleic acid modifying enzyme of this invention refers to the ability of the enzyme to perform its catalytic function under specific reaction conditions. Typically, “efficiency” as defined herein is indicated by the amount of product generated under given reaction conditions.

As used herein, the term “enhances” in the context of an enzyme refers to improving the activity of the enzyme, i.e., increasing the amount of product per unit enzyme per unit time.

An “extendible 3′ end” refers an oligonucleotide with a terminal 3′ nucleotide that may be extended, for example, by a polymerase enzyme, e.g., a 3′ nucleotide that contains a 3′ hydroxyl group.

A “gel” refers to a semi-rigid material permeable to liquids and gases.

The term “hydrogel” herein refers to a macromolecular polymer gel including a network. Within the network, some polymer chains can optionally be cross-linked, although cross-linking does not always occur.

A “gene” refers to a DNA segment that is involved in producing a polypeptide and includes regions preceding and following the coding regions as well as intervening sequences (introns) between individual coding segments (exons).

A “genome” generally refers to genomic information from a subject, which can be, for example, at least a portion of, or the entirety of, the subject's gene-encoded hereditary information. A genome can include coding regions (e.g., that code for proteins) as well as non-coding regions. A genome can include the sequences of some or all of the subject's chromosomes. For example, the human genome ordinarily has a total of 46 chromosomes.

The sequences of some or all of these can constitute the genome.

The terms “hybridizing,” “hybridize,” “annealing,” and “anneal” are used interchangeably in this disclosure and refer to the pairing of substantially complementary or complementary nucleic acid sequences within two different molecules to form a complex. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

As used herein, “hybridizing nucleotide sequence” refers to a nucleotide sequence, within an oligonucleotide for example, that is capable of hybridizing with a complementary nucleotide sequence in a target nucleic acid molecule present on or within a cell from a tissue sample (e.g., cellular RNA, mRNA). When a hybridizing nucleotide sequence is of such a length that it hybridizes with a complementary, either fully or partially, nucleotide sequence that is unique to a target nucleic acid molecule(s) (e.g., cellular RNA or family of RNAs), the hybridizing nucleotide sequence may be said to hybridize to the same target nucleic acid molecule (e.g., mRNA).

As used herein, the term “identical” in the context of two nucleic acids or polypeptide sequences refers to the residues in the two sequences that are the same when aligned for maximum correspondence, as measured using a sequence comparison algorithms. Sequence comparison algorithms are known to those skill in the art. See e.g., ebi.ac.uk/Tools/msa/clustalo/.

When referring to immobilization or attachment of molecules (e.g., nucleic acids) to a support, e.g., a solid support, the terms “immobilized” and “attached” are used interchangeably herein, and both terms are intended to encompass direct or indirect, covalent or non-covalent attachment, unless indicated otherwise. In some embodiments, covalent attachment may be preferred, but generally all that is required is that the molecules (e.g., nucleic acids) remain immobilized or attached to the support under the conditions in which it is intended to use the support, for example in nucleic acid amplification and/or sequencing applications.

As used herein, a “first probe” can refer to a probe that hybridizes to all or a portion of an analyte and can be ligated to one or more additional probes (e.g., a second probe or a spanning probe). In some embodiments, “first probe” can be used interchangeably with “first probe oligonucleotide.”

In some embodiments, the first probe includes ribonucleotides, deoxyribonucleotides, and/or synthetic nucleotides that are capable of participating in Watson-Crick type or analogous base pair interactions. In some embodiments, the first probe includes deoxyribonucleotides. In some embodiments, the first probe includes deoxyribonucleotides and ribonucleotides. In some embodiments, the first probe includes a deoxyribonucleic acid that hybridizes to an analyte and includes a portion of the oligonucleotide that is not a deoxyribonucleic acid. For example, in some embodiments, the portion of the first oligonucleotide that is not a deoxyribonucleic acid is a ribonucleic acid or any other non-deoxyribonucleic acid nucleic acid as described herein. In some embodiments where the first probe includes deoxyribonucleotides, hybridization of the first probe to the mRNA molecule results in a DNA:RNA hybrid. In some embodiments, the first probe includes only deoxyribonucleotides and upon hybridization of the first probe to the mRNA molecule results in a DNA:RNA hybrid.

In some embodiments, the method includes a first probe that includes one or more sequences that are substantially complementary to one or more sequences of an analyte. In some embodiments, a first probe includes a sequence that is substantially complementary to a first target sequence in the analyte. In some embodiments, the sequence of the first probe that is substantially complementary to the first target sequence in the analyte is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the first target sequence in the analyte.

In some embodiments, a first probe includes a sequence that is about 10 nucleotides to about 100 nucleotides (e.g., a sequence of about 10 nucleotides to about 90 nucleotides, about 10 nucleotides to about 80 nucleotides, about 10 nucleotides to about 70 nucleotides, about 10 nucleotides to about 60 nucleotides, about 10 nucleotides to about 50 nucleotides, about 10 nucleotides to about 40 nucleotides, about 10 nucleotides to about 30 nucleotides, about 10 nucleotides to about 20 nucleotides, about 20 nucleotides to about 100 nucleotides, about 20 nucleotides to about 90 nucleotides, about 20 nucleotides to about 80 nucleotides, about 20 nucleotides to about 70 nucleotides, about 20 nucleotides to about 60 nucleotides, about 20 nucleotides to about 50 nucleotides, about 20 nucleotides to about 40 nucleotides, about 20 nucleotides to about 30 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60 nucleotides, about 30 nucleotides to about 50 nucleotides, about 30 nucleotides to about 40 nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50 nucleotides, about 50 nucleotides to about 100 nucleotides, about 50 nucleotides to about 90 nucleotides, about 50 nucleotides to about 80 nucleotides, about 50 nucleotides to about 70 nucleotides, about 50 nucleotides to about 60 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 100 nucleotides, about 70 nucleotides to about 90 nucleotides, about 70 nucleotides to about 80 nucleotides, about 80 nucleotides to about 100 nucleotides, about 80 nucleotides to about 90 nucleotides, or about 90 nucleotides to about 100 nucleotides).

In some embodiments, a first probe includes a functional sequence. In some embodiments, a functional sequence includes a primer sequence. In some embodiments, a first probe includes at least two ribonucleic acid bases at the 3′ end. In such cases, a second probe oligonucleotide comprises a phosphorylated nucleotide at the 5′ end. In some embodiments, a first probe includes at least three, at least four, at least five, at least six, at least seven, at least eight, at least nine, or at least ten ribonucleic acid bases at the 3′ end.

As used herein, a “linker” generally refers to a multifunctional (e.g., bifunctional, trifunctional) reagent used for conjugating two or more chemical moieties.

The terms “linking,” “joining” and “ligation” as used herein, with respect to two polynucleotides, such as an adapter oligonucleotide and a sample polynucleotide or a pair of nucleic acid probes (e.g., hybridized to a nucleic acid template), refers to the covalent attachment of two separate polynucleotides to produce a single larger polynucleotide with a contiguous backbone. In some instances, the nucleic acid probes hybridize adjacent on a nucleic acid template. In other instances, the nucleic acid probes hybridize non-adjacently on a nucleic acid template, wherein the 3-end of one of the probes is then extended until it is adjacently positioned to the 5′ end of the other probes prior to ligation.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

As used herein, the term “mutation” or “mutant” or “variant” indicates a change or changes introduced in a wildtype DNA sequence or a wild-type amino acid sequence. Examples of mutations or variants include, but are not limited to, substitutions, insertions, deletions, and point mutations. Mutations can be made either at the nucleic acid level or at the amino acid level.

As used herein, the term “operably linked” or “conjugated” or “fusion” means that, in relation to the recombinant thermostable polymerase enzyme sequence there are one or more sequences at the N or C terminus that, when transcribed and translated, create additional polypeptides in association with the enzyme amino acid sequence, thereby created a conjugation or fusion of one or more polypeptides from one expression vector.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The term “partitioning” as used herein is intended to encompass parting, dividing, depositing, separating, or compartmentalizing into one or more partitions. Systems and methods for partitioning of one or more particles (such as, but not limited to, biological particles, macromolecular constituents of biological particles, beads, reagents, etc.) into discrete compartments or partitions (referred to interchangeably here as partitions), wherein each partition maintains separation of its own content from the contents of other partitions are known in the art. See for example US 2020/0032335, herein incorporated by reference in its entirety. The partition can be a droplet in an emulsion. A partition may comprise one or more other partitions.

In some embodiments, methods provided herein include use of the chimeric ligase enzymes for simultaneous ligation of probes to a plurality of nucleic acid template molecules. In some embodiments, the method involves simultaneous ligation of probes to at least about 100 nucleic acid template molecules, at least about 500 nucleic acid template molecules, at least about 1,000 nucleic acid templates molecules, at least about 5,000 nucleic acid template molecules, or at least about 10,000 nucleic acid template molecules.

In some embodiments, methods provided herein include use of chimeric ligase enzymes for ligation of ligateable probes to nucleic acid template molecules, wherein the ligateable probes include a partition-specific barcode sequence or the ligateable probes are capable of hybridizing to an oligonucleotide including a partition-specific barcode. In some embodiments, the ligateable probes include a unique molecular identifier (UMI) sequence, or the ligateable probes are capable of hybridizing to an oligonucleotide including a UMI sequence. In some embodiments, the methods involve ligation of ligateable probes, wherein the ligateable probes include a sequence that is capable of hybridizing to an oligonucleotide that includes a partition-specific sequence and a UMI.

The term polymerase encompasses DNA polymerases, RNA polymerases, and reverse transcriptases. A “DNA polymerase” catalyzes the polymerization of deoxyribonucleotides.

As used herein, “polypeptide” refers to a composition comprised of amino acids and recognized as a protein by those of skill in the art. The conventional one-letter or three-letter code for amino acid residues is used herein. The terms “polypeptide” and “protein” are used interchangeably herein to refer to polymers of amino acids of any length. The polymer may be linear or branched, it may include modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

A “probe,” when used in reference to a nucleic acid or sequence of a nucleic acids, is intended as a semantic identifier for the nucleic acid or sequence in the context of a method or composition and does not limit the structure or function of the nucleic acid or sequence beyond what is expressly indicated. In one example, one or more probes can be hybridized to a nucleic acid template to form a complex (e.g., a duplex) comprising one or more probes and the nucleic acid template.

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

As used herein, the term “reverse transcriptase activity,” “reverse transcription activity,” or “reverse transcription” indicates the capability of an enzyme to synthesize a DNA strand (that is, complementary DNA or cDNA) using RNA as a template.

In some embodiments, provided herein are chimeric ligases including an RNAse H domain that has reduced catalytic activity or RNAse activity. RNAse catalytic activity includes hydrolyzing the RNA in RNA/DNA hybrid. Many reverse transcriptases have an RNAseH activity that can be inactivated by truncation or by substitution. A reduction in RNAseH activity may for example be a 50% or 75% reduction, or may be a 100% reduction (i.e., loss) of RNAseH activity. RNAseH activity can be abolished by an amino acid substitution (see e.g., Kotewicz et al., Nucleic Acids Res. 1988 16: 265-77 and Schultz et al., J. Virol. 1996 70: 8630-8, among others).

As used herein, a “second probe” can refer to a probe that hybridizes to all or a portion of an analyte and can be ligated to one or more additional probes (e.g., a first probe or a spanning probe). In some embodiments, “second probe” can be used interchangeably with “second probe oligonucleotide.” One of skill in the art will appreciate that the order of the probes is arbitrary, and thus the contents of the first probe and/or second probe as disclosed herein are interchangeable.

In some embodiments, the second probe includes ribonucleotides, deoxyribonucleotides, and/or synthetic nucleotides that are capable of participating in Watson-Crick type or analogous base pair interactions. In some embodiments, the second probe includes deoxyribonucleotides. In some embodiments, the second probe includes deoxyribonucleotides and ribonucleotides. In some embodiments, the second probe includes a deoxyribonucleic acid that hybridizes to an analyte and includes a portion of the oligonucleotide that is not a deoxyribonucleic acid. For example, in some embodiments, the portion of the second probe that is not a deoxyribonucleic acid is a ribonucleic acid or any other non-deoxyribonucleic acid nucleic acid as described herein. In some embodiments where the second probe includes deoxyribonucleotides, hybridization of the second probe to the mRNA molecule results in a DNA:RNA hybrid. In some embodiments, the second probe includes only deoxyribonucleotides and upon hybridization of the first probe to the mRNA molecule results in a DNA:RNA hybrid.

In some embodiments, the method includes a second probe that includes one or more sequences that are substantially complementary to one or more sequences of an analyte. In some embodiments, a second probe includes a sequence that is substantially complementary to a second target sequence in the analyte. In some embodiments, the sequence of the second probe that is substantially complementary to the second target sequence in the analyte is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the second target sequence in the analyte.

In some embodiments, a second probe includes a sequence that is about 10 nucleotides to about 100 nucleotides (e.g., a sequence of about 10 nucleotides to about 90 nucleotides, about 10 nucleotides to about 80 nucleotides, about 10 nucleotides to about 70 nucleotides, about 10 nucleotides to about 60 nucleotides, about 10 nucleotides to about 50 nucleotides, about 10 nucleotides to about 40 nucleotides, about 10 nucleotides to about 30 nucleotides, about 10 nucleotides to about 20 nucleotides, about 20 nucleotides to about 100 nucleotides, about 20 nucleotides to about 90 nucleotides, about 20 nucleotides to about 80 nucleotides, about 20 nucleotides to about 70 nucleotides, about 20 nucleotides to about 60 nucleotides, about 20 nucleotides to about 50 nucleotides, about 20 nucleotides to about 40 nucleotides, about 20 nucleotides to about 30 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60 nucleotides, about 30 nucleotides to about 50 nucleotides, about 30 nucleotides to about 40 nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50 nucleotides, about 50 nucleotides to about 100 nucleotides, about 50 nucleotides to about 90 nucleotides, about 50 nucleotides to about 80 nucleotides, about 50 nucleotides to about 70 nucleotides, about 50 nucleotides to about 60 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 100 nucleotides, about 70 nucleotides to about 90 nucleotides, about 70 nucleotides to about 80 nucleotides, about 80 nucleotides to about 100 nucleotides, about 80 nucleotides to about 90 nucleotides, or about 90 nucleotides to about 100 nucleotides).

In some embodiments, a second probe includes a phosphorylated nucleotide at the 5′ end. The phosphorylated nucleotide at the 5′ end can be used in a ligation reaction to ligate the second probe to the first probe.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent@). Alternatively, or in addition, sequencing may be performed using nucleic acid amplification, polymerase chain reaction (PCR) (e.g., digital PCR, quantitative PCR, or real time PCR), or isothermal amplification.

Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

A “splint oligonucleotide” is an oligonucleotide that, when hybridized to other polynucleotides, acts as a “splint” to position the polynucleotides next to one another so that they can be ligated together. In some embodiments, the splint oligonucleotide is DNA or RNA. The splint oligonucleotide can include a nucleotide sequence that is partially complimentary to nucleotide sequences from two or more different oligonucleotides. In some embodiments, the splint oligonucleotide assists in ligating a “donor” oligonucleotide and an “acceptor” oligonucleotide. In general, an RNA ligase, a DNA ligase, or another other variety of ligase is used to ligate two nucleotide sequences together, for example the ligase of the present disclosure.

In some embodiments, the splint oligonucleotide is between 10 and 50 oligonucleotides in length, e.g., between 10 and 45, 10 and 40, 10 and 35, 10 and 30, 10 and 25, or 10 and 20 oligonucleotides in length. In some embodiments, the splint oligonucleotide is between 15 and 50, 15 and 45, 15 and 40, 15 and 35, 15 and 30, 15 and 30, or 15 and 25 nucleotides in length.

As used herein, the term “strand-displacing polymerase”, refers to a polymerase that is able to displace one or more nucleotides, such as at least 10 or 100 or more nucleotides that are downstream from the enzyme. Strand displacing polymerases can be differentiated from non-strand displacing polymerase. In some embodiments, the strand displacing polymerase is stable and active at a temperature of at least 50° C. or at least 55° C. (including the strand displacing activity). Taq polymerase is a nick translating polymerase and, as such, is not a strand displacing polymerase.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

In some embodiments, a “subject” or “individual” can refer to the source from which a biological sample is obtained, for example, but not limited to, a mammal (e.g., human or a non-human simian), or avian (e.g., bird), or other organism, such as a plant. Examples of subjects include, but are not limited to, a mammal such as a rodent, mouse, rat, rabbit, guinea pig, ungulate, horse, sheep, pig, goat, cow, cat, dog, primate (i.e., human or non-human primate); a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, or honey bee; an arachnid such as a spider; a fish such as zebrafish; a reptile; an amphibian such as a frog or Xenopus laevis; a Dictyostelium discoideum; a fungi such as Pneumocystis carinii, Takifugu rubripes, yeast, Saccharamoyces cerevisiae or Schizosaccharomyces pombe; or a Plasmodium falciparum:.

The phrases “substantially similar” and “substantially identical” in the context of at least two nucleic acids typically means that a polynucleotide includes a sequence that has at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% sequence identity, in comparison with a reference (e.g., wild-type) polynucleotide or polypeptide. Sequence identity may be determined using known programs such as BLAST, ALIGN, and CLUSTAL using standard parameters. See e.g., Altshul et al. (1990) J. Mol. Biol. 215:403-410; Henikoff et al. (1989) Proc. Natl. Acad. Sci. 89:10915; Karlin et al. (1993) Proc. Natl. Acad. Sci. 90:5873; and Higgins et al. (1988) Gene 73:237. Software for performing BLAST analyses is publicly available through the National Center for Biotechnology Information. Also, databases may be searched using FASTA (Pearson et al. (1988) Proc. Natl. Acad. Sci. 85:2444-2448.) In some embodiments, substantially identical nucleic acid molecules hybridize to each other under stringent conditions (e.g., within a range of medium to high stringency).

A “substrate” is a support that is insoluble in aqueous liquid and which allows for positioning of biological samples, analytes, features, and/or capture probes on the substrate. Further, a “substrate” as used herein, and when not preceded by the modifier “chemical,” refers to a member with at least one surface that generally functions to provide physical support for biological samples, analytes, and/or any of the other chemical and/or physical moieties, agents, and structures described herein.

Nucleic acid “synthesis” herein refers to any in vitro method for making a new strand of polynucleotide or elongating an existing polynucleotide (i.e., DNA or RNA) in a template dependent manner. Synthesis, according to the invention, can include amplification, which increases the number of copies of a polynucleotide template sequence with the use of a polymerase. Polynucleotide synthesis (e.g., amplification) results in the incorporation of nucleotides into a polynucleotide (e.g., extension from a primer), thereby forming a new polynucleotide molecule complementary to the polynucleotide template. The formed polynucleotide molecule and its template can be used as templates to synthesize additional polynucleotide molecules. “DNA synthesis,” as used herein, includes, but is not limited to, polymerase chain reaction (PCR), and may include the use of labeled nucleotides, e.g., for probes and oligonucleotide primers, or for polynucleotide sequencing.

A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of a complementary region between a template strand and two substrate ends to be ligated.

The term “tag” may refer to a detectable moiety that may be one or more atom(s) or molecule(s), or a collection of atoms and molecules. A tag may provide an optical, electrochemical, magnetic, or electrostatic (e.g., inductive, capacitive) signature.

The term “tagged nucleotide” herein refers to a nucleotide that includes a tag (or tag species) that is coupled to any location of the nucleotide including, but not limited to a phosphate (e.g., terminal phosphate), sugar or nitrogenous base moiety of the nucleotide. Tags may be one or more atom(s) or molecule(s), or a collection of atoms and molecules. A tag may provide an optical, electrochemical, magnetic, or electrostatic (e.g., inductive, capacitive) signature.

As used herein, the term “target polynucleotide” refers to a nucleic acid molecule or polynucleotide in a population of nucleic acid molecules having a target sequence to which one or more oligonucleotides are designed to hybridize. In some embodiments, a target sequence uniquely identifies a sequence derived from a sample, such as a particular genomic, mitochondrial, bacterial, viral, or RNA (e.g., mRNA, miRNA, primary miRNA, or pre-miRNA) sequence. In some embodiments, a target sequence is a common sequence shared by multiple different target polynucleotides, such as a common adapter sequence joined to different target polynucleotides. “Target polynucleotide” may be used to refer to a double-stranded nucleic acid molecule that includes a target sequence on one or both strands, or a single-stranded nucleic acid molecule including a target sequence and may be derived from any source of or process for isolating or generating nucleic acid molecules (e.g., a target polynucleotide may be derived from or originate from a sample and may be referred to as a sample polynucleotide). A target polynucleotide may include one or more (e.g., 1, 2, 3, 4, 5, 6, 7, 8, 9, 10, or more) target sequences, which may be the same or different. In general, different target polynucleotides include different sequences, such as one or more different nucleotides or one or more different target sequences.

The term “template DNA molecule” herein refers to a strand of a nucleic acid from which a complementary nucleic acid strand is synthesized by a DNA polymerase, for example, in a primer extension reaction.

The term “template RNA molecule” herein refers to a strand of a ribonucleic acid (i) from which a complementary nucleic acid strand (cDNA) is synthesized by a DNA polymerase, for example, in a primer extension reaction or (ii) to which two probes are hybridized to serve as a substrate for templated ligation, for example in an RNA templated ligation reaction.

The term “template-dependent manner” refers to a process that involves the template dependent extension of a primer molecule (e.g., DNA synthesis by DNA polymerase). The term “template-dependent manner” typically refers to polynucleotide synthesis of RNA or DNA wherein the sequence of the newly synthesized strand of polynucleotide is dictated by the well-known rules of complementary base pairing (see, for example, Watson, J. D. et al., In: Molecular Biology of the Gene, 4th Ed., W. A. Benjamin, Inc., Menlo Park, Calif. (1987)).

As used herein, the term “thermoreactivity” or “thermoreactive” may refer to the ability of a reverse transcriptase to exhibit enzyme activity at elevated temperatures.

As used herein, “thermostability” or “thermostable” may refer to the ability of a reverse transcriptase to withstand exposure to elevated temperatures, but not necessarily show activity at such elevated temperatures. In some embodiments, thermostable reverse transcriptase (e.g., the engineered enzyme) or polymerase refers to any enzyme that catalyzes polynucleotide synthesis by addition of nucleotide units to a nucleotide chain using DNA or RNA as a template and has an optimal activity at a temperature above 53° C.

As used herein, the terms “unique molecular identifier,” “unique molecular identifying sequence,” “UMI” and “UMI sequence” are used synonymously. Individual barcoded molecules may comprise a common barcode sequence such as a partition specific sequence or a spatial array where every capture probe has a unique barcode sequence.

As used herein, the term “Variant” means a protein which is derived from a precursor protein (such as the native protein, for example MMLV native protein as set forth in SEQ ID NO: 15) by addition of one or more amino acids to either or both the C- and N-terminal end, substitution of one or more amino acids at one or a number of different sites in the amino acid sequence, deletion of one or more amino acids at either or both ends of the protein or at one or more sites in the amino acid sequence, or addition of a fusion domain. For example, SEQ ID NO: 14 is a variant of MMLV of SEQ ID NO: 15. The preparation of an enzyme variant is preferably achieved by modifying a DNA sequence which encodes for the wild-type protein, transformation of that DNA sequence into a suitable host, and expression of the modified DNA sequence to form the derivative enzyme. A variant ligase or RNAseH of the present disclosure includes a protein comprising altered amino acid sequences in comparison with a precursor enzyme amino acid sequence wherein the variant reverse transcriptase retains the characteristic enzymatic nature of the precursor enzyme but which may have altered properties in some specific aspect. For example, the chimeric ligase polypeptide variant may have an altered pH optimum or increased temperature stability but may retain its characteristic ligase activity.

A “Variant” may have at least about 45%, at least about 50%, at least about 55%, at least about 60%, at least about 65%, at least about 70%, at least about 75%, at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, or at least about 99.5% sequence identity to a polypeptide sequence when optimally aligned for comparison. Percent identity may pertain to the percent identity of the DNA binding domain or the engineered reverse transcriptase portion of an engineered reverse transcriptase. As used herein, a variant residue position is described in relation to the wild-type or precursor amino acid sequence set forth in SEQ ID NO: 15; the amino acid position is indexed to SEQ ID NO: 14. A fusion variant comprises at least one fusion domain selected from RNAseH domains or ligase described elsewhere herein.

As used herein, a protein having a certain percent (e.g., at least 90%, at least 91%, at least 92%, at least 93%, at least 94%, at least 95%, at least 96%, at least 97%, at least 98%, or at least 99%) of sequence identity with another sequence means that, when aligned, that percentage of bases or amino acid residues are the same in comparing the two sequences. This alignment and the percent homology or identity can be determined using any suitable software program known in the art, for example those described in CURRENT PROTOCOLS IN MOLECULAR BIOLOGY, Ausubel et al., eds., 1987, Supplement 30, section 7.7.18. Representative programs include the Vector NTI Advance™ 9.0 (Invitrogen Corp. Carlsbad, CA), GCG Pileup, FASTA (Pearson et al. (1988) Proc. Natl Acad. Sci USA 85:2444-2448), and BLAST (BLAST Manual, Altschul et al., Nat'l Cent. Biotechnol. Inf, Nat'l Lib. Med. (NCIB NLM NIH), Bethesda, Md., and Altschul et al., (1997) Nucleic Acids Res. 25:3389-3402) programs. Another typical alignment program is ALIGN Plus (Scientific and Educational Software, PA), generally using default parameters. Other sequence alignment software programs that find use are the TFASTA Data Searching Program available in the Sequence Software Package Version 6.0 (Genetics Computer Group, University of Wisconsin, Madison, WI and CLC Main Workbench (Qiagen) Version 20.0. The present disclosure is not limited to the software being used to align two or more sequences.

As used herein, the term “wild-type” refers to a gene or gene product that has the characteristics of that gene or gene product when isolated from a naturally occurring source.

The amino acid sequence set forth in SEQ ID NO: 15 is a wild-type (wt) Murine Moloney Leukemia Virus (MMLV) sequence (Genbank NP_955591.1 p80 RT).

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention which can be had by reference to the specification as a whole. Accordingly, the terms defined immediately below are more fully defined by reference to the specification as a whole.

TABLE 3
Sequences

| SEQ | | |
|---|---|---|
| ID | | |
| NO | Description | Sequences |
| 1 | LigAT_C- | ATGGCTATTCAGAAGCCGTTACTGGCGGCCAGC |
| | RNAseH-E. coli | CTGAAGAAAATGTCTGTTGACAACTTGACTTTC |
| | Nucleic Acid | CCGGTCTACGCAACGCCGAAACTGGACGGTATC |
| | | CGTGCGCTGAAGATTGACGGCACCCTGGTGTCG |
| | | CGTACGTTCAAACCGATCCGCAATACTACCATC |
| | | TCCAAGGTCCTGGCGAGCTTGCTGCCGGACGGC |
| | | AGCGATGGTGAGATCTTGTCTGGTAAGACCTTT |
| | | CAGGATAGCACCAGCACCGTGATGACGACCGA |
| | | TGCGGGTATCGGTAGCGATACCACCTTCTTCTG |
| | | GTTTGATTACGTTAAAGACGACCCGGATAAGGG |
| | | TTACCTGGACCGTATCGCTGACATGAAAACCTT |
| | | CGTCGACCAGCACCCGGAAATCCTGAAAGACA |
| | | GCTGCGTGACGATCGTCCCGCTGTTCCCGAAGA |
| | | AAATTGACACGCCAGAAGAACTGCATGTGTTTG |
| | | AGAAATGGTGTCTGGACCAGGGTTTTGAGGGCG |
| | | TTATGGTGCGTACGGCCGGTGGCAAGTATAAGT |
| | | TTGGTCGCAGCACCGAGAAAGAACAAATCCTG |
| | | GTTAAGATTAAACAGTTTGAAGATGATGAAGCT |
| | | GTTGTGATCGGTGTGAGCGCGCTGCAAACCAAT |
| | | ACGAACGATAAGAAACTGAATCAGCTGGGTGA |
| | | GATGCGTCGTACTAGCCACCAAGACGGCAAAGT |
| | | TGACCTGGACATGCTGGGTGCGCTTGACGTGGA |
| | | CTGGAATGGTATTCGTTTCAGCATCGGTACGGG |
| | | CTTTGATAAAGACACCCGCGAAGATCTCTGGAA |
| | | ACAACGCGACAGCATCGTGGGTAAAGTCGTTAA |
| | | GTTCAAGTATTTCTCCCAGGGTATTAAGACCGC |
| | | TCCGCGTTTTCCTGTGTTTCTGGGTTTTCGTGAC |
| | | GAGAACGATATGGGTGGCAGCGGTGGCAGCA |
| | | TGCTGAAGCAGGTGGAAATCTTTACAAACGG |
| | | TTCTTGTCTGGGAAATCCTGGACCCGGAGGA |
| | | TATGGTGCAATTTTGCGTTATCGCGGACGCG |
| | | AAAAGACATTTTCAGCGGGATATACTCGTAC |
| | | AACGAACAACCGCATGGAACTTATGGCCGCA |
| | | ATCGTGGCACTTGAGGCGCTGAAGGAACATT |
| | | GCGAAGTGATCCTGTCTACAGATAGTCAATA |
| | | TGTGCGCCAAGGAATTACGCAGTGGATCCAT |
| | | AATTGGAAGAAGCGTGGCTGGAAGACCGCA |
| | | GACAAAAAACCAGTGAAAAACGTAGACTTAT |
| | | GGCAACGTCTGGATGCTGCTCTGGGACAACA |
| | | CCAAATCAAATGGGAATGGGTGAAGGGACAT |
| | | GCCGGACATCCCGAGAATGAGCGTTGTGACG |
| | | AATTAGCGCGTGCCGCCGCAATGAATCCTAC |
| | | GCTGGAAGACACCGGTTATCAGGTGGAGGTT |
| | | TAA |
| 2 | LigAT_C- | MAIQKPLLAASLKKMSVDNLTFPVYATPKLDGIR |
| | RNAseH-Ecoli | ALKIDGTLVSRTFKPIRNTTISKVLASLLPDGSDGEI |
| | Amino Acid | LSGKTFQDSTSTVMTTDAGIGSDTTFFWFDYVKD |
| | | DPDKGYLDRIADMKTFVDQHPEILKDSCVTIVPLF |
| | | PKKIDTPEELHVFEKWCLDQGFEGVMVRTAGGK |
| | | YKFGRSTEKEQILVKIKQFEDDEAVVIGVSALQTN |
| | | TNDKKLNQLGEMRRTSHQDGKVDLDMLGALDV |
| | | DWNGIRFSIGTGFDKDTREDLWKQRDSIVGKVVK |
| | | FKYFSQGIKTAPRFPVFLGFRDENDMGGSGGSML |
| | | KQVEIFTNGSCLGNPGPGGYGAILRYRGREKTF |
| | | SAGYTRTTNNRMELMAAIVALEALKEHCEVIL |
| | | STDSQYVRQGITQWIHNWKKRGWKTADKKPV |
| | | KNVDLWQRLDAALGQHQIKWEWVKGHAGHP |
| | | ENERCDELARAAAMNPTLEDTGYQVEV |
| 3 | LigAT_N- | ATGCTGAAGCAGGTGGAAATCTTTACAAACG |
| | RNAseH-Ecoli | GTTCTTGTCTGGGAAATCCTGGACCCGGAGG |
| | Nucleic acid | ATATGGTGCAATTTTGCGTTATCGCGGACGC |
| | | GAAAAGACATTTTCAGCGGGATATACTCGTA |
| | | CAACGAACAACCGCATGGAACTTATGGCCGC |
| | | AATCGTGGCACTTGAGGCGCTGAAGGAACAT |
| | | TGCGAAGTGATCCTGTCTACAGATAGTCAAT |
| | | ATGTGCGCCAAGGAATTACGCAGTGGATCCA |
| | | TAATTGGAAGAAGCGTGGCTGGAAGACCGCA |
| | | GACAAAAAACCAGTGAAAAACGTAGACTTAT |
| | | GGCAACGTCTGGATGCTGCTCTGGGACAACA |
| | | CCAAATCAAATGGGAATGGGTGAAGGGACAT |
| | | GCCGGACATCCCGAGAATGAGCGTTGTGACG |
| | | AATTAGCGCGTGCCGCCGCAATGAATCCTAC |
| | | GCTGGAAGACACCGGTTATCAGGTGGAGGTT |
| | | GGTTCAGGGGGTTCCGGTATGGCTATTCAGAA |
| | | GCCGTTACTGGCGGCCAGCCTGAAGAAAATGTC |
| | | TGTTGACAACTTGACTTTCCCGGTCTACGCAAC |
| | | GCCGAAACTGGACGGTATCCGTGCGCTGAAGAT |
| | | TGACGGCACCCTGGTGTCGCGTACGTTCAAACC |
| | | GATCCGCAATACTACCATCTCCAAGGTCCTGGC |
| | | GAGCTTGCTGCCGGACGGCAGCGATGGTGAGAT |
| | | CTTGTCTGGTAAGACCTTTCAGGATAGCACCAG |
| | | CACCGTGATGACGACCGATGCGGGTATCGGTAG |
| | | CGATACCACCTTCTTCTGGTTTGATTACGTTAAA |
| | | GACGACCCGGATAAGGGTTACCTGGACCGTATC |
| | | GCTGACATGAAAACCTTCGTCGACCAGCACCCG |
| | | GAAATCCTGAAAGACAGCTGCGTGACGATCGTC |
| | | CCGCTGTTCCCGAAGAAAATTGACACGCCAGAA |
| | | GAACTGCATGTGTTTGAGAAATGGTGTCTGGAC |
| | | CAGGGTTTTGAGGGCGTTATGGTGCGTACGGCC |
| | | GGTGGCAAGTATAAGTTTGGTCGCAGCACCGAG |
| | | AAAGAACAAATCCTGGTTAAGATTAAACAGTTT |
| | | GAAGATGATGAAGCTGTTGTGATCGGTGTGAGC |
| | | GCGCTGCAAACCAATACGAACGATAAGAAACT |
| | | GAATCAGCTGGGTGAGATGCGTCGTACTAGCCA |
| | | CCAAGACGGCAAAGTTGACCTGGACATGCTGG |
| | | GTGCGCTTGACGTGGACTGGAATGGTATTCGTT |
| | | TCAGCATCGGTACGGGCTTTGATAAAGACACCC |
| | | GCGAAGATCTCTGGAAACAACGCGACAGCATC |
| | | GTGGGTAAAGTCGTTAAGTTCAAGTATTTCTCC |
| | | CAGGGTATTAAGACCGCTCCGCGTTTTCCTGTG |
| | | TTTCTGGGTTTTCGTGACGAGAACGATATGTAA |
| 4 | LigAT_N- | MLKQVEIFTNGSCLGNPGPGGYGAILRYRGRE |
| | RNAseH-Ecoli | KTFSAGYTRTTNNRMELMAAIVALEALKEHCE |
| | Amino acid | VILSTDSQYVRQGITQWIHNWKKRGWKTADK |
| | | KPVKNVDLWQRLDAALGQHQIKWEWVKGHA |
| | | GHPENERCDELARAAAMNPTLEDTGYQVEVGS |
| | | GGSGMAIQKPLLAASLKKMSVDNLTFPVYATPKL |
| | | DGIRALKIDGTLVSRTFKPIRNTTISKVLASLLPDG |
| | | SDGEILSGKTFQDSTSTVMTTDAGIGSDTTFFWFD |
| | | YVKDDPDKGYLDRIADMKTFVDQHPEILKDSCVT |
| | | IVPLFPKKIDTPEELHVFEKWCLDQGFEGVMVRTA |
| | | GGKYKFGRSTEKEQILVKIKQFEDDEAVVIGVSAL |
| | | QTNTNDKKLNQLGEMRRTSHQDGKVDLDMLGA |
| | | LDVDWNGIRFSIGTGFDKDTREDLWKQRDSIVGK |
| | | VVKFKYFSQGIKTAPRFPVFLGFRDENDM |
| 5 | LigAT_C- | ATGGCTATTCAGAAGCCGTTACTGGCGGCCAGC |
| | RNAseH-Sto7 | CTGAAGAAAATGTCTGTTGACAACTTGACTTTC |
| | Nucleic Acid | CCGGTCTACGCAACGCCGAAACTGGACGGTATC |
| | | CGTGCGCTGAAGATTGACGGCACCCTGGTGTCG |
| | | CGTACGTTCAAACCGATCCGCAATACTACCATC |
| | | TCCAAGGTCCTGGCGAGCTTGCTGCCGGACGGC |
| | | AGCGATGGTGAGATCTTGTCTGGTAAGACCTTT |
| | | CAGGATAGCACCAGCACCGTGATGACGACCGA |
| | | TGCGGGTATCGGTAGCGATACCACCTTCTTCTG |
| | | GTTTGATTACGTTAAAGACGACCCGGATAAGGG |
| | | TTACCTGGACCGTATCGCTGACATGAAAACCTT |
| | | CGTCGACCAGCACCCGGAAATCCTGAAAGACA |
| | | GCTGCGTGACGATCGTCCCGCTGTTCCCGAAGA |
| | | AAATTGACACGCCAGAAGAACTGCATGTGTTTG |
| | | AGAAATGGTGTCTGGACCAGGGTTTTGAGGGCG |
| | | TTATGGTGCGTACGGCCGGTGGCAAGTATAAGT |
| | | TTGGTCGCAGCACCGAGAAAGAACAAATCCTG |
| | | GTTAAGATTAAACAGTTTGAAGATGATGAAGCT |
| | | GTTGTGATCGGTGTGAGCGCGCTGCAAACCAAT |
| | | ACGAACGATAAGAAACTGAATCAGCTGGGTGA |
| | | GATGCGTCGTACTAGCCACCAAGACGGCAAAGT |
| | | TGACCTGGACATGCTGGGTGCGCTTGACGTGGA |
| | | CTGGAATGGTATTCGTTTCAGCATCGGTACGGG |
| | | CTTTGATAAAGACACCCGCGAAGATCTCTGGAA |
| | | ACAACGCGACAGCATCGTGGGTAAAGTCGTTAA |
| | | GTTCAAGTATTTCTCCCAGGGTATTAAGACCGC |
| | | TCCGCGTTTTCCTGTGTTTCTGGGTTTTCGTGAC |
| | | GAGAACGATATGGGTGGCAGCGGTGGCAGCA |
| | | TGATCATTGGTTACTTCAACGGGCTTTGCGA |
| | | ACCTAAAAACCCCGGCGGTATTGCGACCTTT |
| | | GGCTTCGTCATTTACCTGGATAACCGCAAAA |
| | | TCGAGGGCTATGGTTTAGCCGAGAAGCCATT |
| | | CTCTATCAACTCCACCAATAATGTTGCCGAG |
| | | TATTCCGGTTTGATCTGCCTTATGGAGACAA |
| | | TGTTACGCTTAGGAATTAGTAGTCCCATTATT |
| | | AAAGGTGACTCACAATTGGTTATCAAACAGA |
| | | TGAATGGCGAGTACAAGGTCAAAGCGAAACG |
| | | TATTATCCCTTTATACGAGAAAGCTATTGAAT |
| | | TGAAAAAGAAGTTGAATGCCACCCTTATTTG |
| | | GGTGCCTCGCGAGGAGAATAAGGAGGCTGA |
| | | TCGTTTGTCTCGTGTCGCCTATGAGTTGGTG |
| | | CGTCGCGGCAAATTGCGTGACATTGGGTGCA |
| | | TTATTCTGACGTAA |
| 6 | LigAT_C- | MAIQKPLLAASLKKMSVDNLTFPVYATPKLDGIR |
| | RNAseH-Sto7 | ALKIDGTLVSRTFKPIRNTTISKVLASLLPDGSDGEI |
| | Amino Acid | LSGKTFQDSTSTVMTTDAGIGSDTTFFWFDYVKD |
| | | DPDKGYLDRIADMKTFVDQHPEILKDSCVTIVPLF |
| | | PKKIDTPEELHVFEKWCLDQGFEGVMVRTAGGK |
| | | YKFGRSTEKEQILVKIKQFEDDEAVVIGVSALQTN |
| | | TNDKKLNQLGEMRRTSHQDGKVDLDMLGALDV |
| | | DWNGIRFSIGTGFDKDTREDLWKQRDSIVGKVVK |
| | | FKYFSQGIKTAPRFPVFLGFRDENDMGGSGGSMII |
| | | GYFNGLCEPKNPGGIATFGFVIYLDNRKIEGYG |
| | | LAEKPFSINSTNNVAEYSGLICLMETMLRLGISS |
| | | PIIKGDSQLVIKQMNGEYKVKAKRIIPLYEKAIE |
| | | LKKKLNATLIWVPREENKEADRLSRVAYELVR |
| | | RGKLRDIGCIILT |
| 7 | LigAT_C- | ATGGCTATTCAGAAGCCGTTACTGGCGGCCAGC |
| | RNAseH-RT | CTGAAGAAAATGTCTGTTGACAACTTGACTTTC |
| | Nucleic Acid | CCGGTCTACGCAACGCCGAAACTGGACGGTATC |
| | | CGTGCGCTGAAGATTGACGGCACCCTGGTGTCG |
| | | CGTACGTTCAAACCGATCCGCAATACTACCATC |
| | | TCCAAGGTCCTGGCGAGCTTGCTGCCGGACGGC |
| | | AGCGATGGTGAGATCTTGTCTGGTAAGACCTTT |
| | | CAGGATAGCACCAGCACCGTGATGACGACCGA |
| | | TGCGGGTATCGGTAGCGATACCACCTTCTTCTG |
| | | GTTTGATTACGTTAAAGACGACCCGGATAAGGG |
| | | TTACCTGGACCGTATCGCTGACATGAAAACCTT |
| | | CGTCGACCAGCACCCGGAAATCCTGAAAGACA |
| | | GCTGCGTGACGATCGTCCCGCTGTTCCCGAAGA |
| | | AAATTGACACGCCAGAAGAACTGCATGTGTTTG |
| | | AGAAATGGTGTCTGGACCAGGGTTTTGAGGGCG |
| | | TTATGGTGCGTACGGCCGGTGGCAAGTATAAGT |
| | | TTGGTCGCAGCACCGAGAAAGAACAAATCCTG |
| | | GTTAAGATTAAACAGTTTGAAGATGATGAAGCT |
| | | GTTGTGATCGGTGTGAGCGCGCTGCAAACCAAT |
| | | ACGAACGATAAGAAACTGAATCAGCTGGGTGA |
| | | GATGCGTCGTACTAGCCACCAAGACGGCAAAGT |
| | | TGACCTGGACATGCTGGGTGCGCTTGACGTGGA |
| | | CTGGAATGGTATTCGTTTCAGCATCGGTACGGG |
| | | CTTTGATAAAGACACCCGCGAAGATCTCTGGAA |
| | | ACAACGCGACAGCATCGTGGGTAAAGTCGTTAA |
| | | GTTCAAGTATTTCTCCCAGGGTATTAAGACCGC |
| | | TCCGCGTTTTCCTGTGTTTCTGGGTTTTCGTGAC |
| | | GAGAACGATATGGGTGGCAGCGGTGGCAGCA |
| | | TCCTGGCAGAGGCGCACGGCACTCGCCCGG |
| | | ATCTGACGGACCAGCCGCTGCCGGACGCCGA |
| | | TCATACCTGGTATACGAATGGTAGCAGCCTG |
| | | TTGCAAGAGGGTCAGCGTAAGGCCGGTGCC |
| | | GCGGTCACCACCGAGACTGAAGTGATTTGGG |
| | | CTAAAGCATTGCCTGCGGGTACCAGCGCGCA |
| | | GCGTGCCGAGCTGATCGCACTGACCCAAGCG |
| | | CTGAAAATGGCTGAGGGTAAGAAACTGAATG |
| | | TGTACACGGATAGCCGTTATGCCTTTGCGAC |
| | | CGCCCACATTCACGGCGAGATCTATCGCCGT |
| | | CGCGGCTGGCTGACGTCCAAAGGCAAAGAG |
| | | ATCAAGAATAAAGACGAAATTCTGGCGCTGC |
| | | TGAAAGCGCTGTTCCTGCCGAAACGTCTGTC |
| | | GATCATCCATTGCCCGGGTCACCAGAAAGGC |
| | | CACAGCGCAGAGGCGCGTGGTAATCGCATG |
| | | GCTGACCAGGCTGCGCGTAAAGCCGCAATTA |
| | | CCGAAACCCCGGACACCAGCACGCTGCTGTA |
| | | A |
| 8 | LigAT_C- | MAIQKPLLAASLKKMSVDNLTFPVYATPKLDGIR |
| | RNAseH-RT | ALKIDGTLVSRTFKPIRNTTISKVLASLLPDGSDGEI |
| | Amino Acid | LSGKTFQDSTSTVMTTDAGIGSDTTFFWFDYVKD |
| | | DPDKGYLDRIADMKTFVDQHPEILKDSCVTIVPLF |
| | | PKKIDTPEELHVFEKWCLDQGFEGVMVRTAGGK |
| | | YKFGRSTEKEQILVKIKQFEDDEAVVIGVSALQTN |
| | | TNDKKLNQLGEMRRTSHQDGKVDLDMLGALDV |
| | | DWNGIRFSIGTGFDKDTREDLWKQRDSIVGKVVK |
| | | FKYFSQGIKTAPRFPVFLGFRDENDMGGSGGSILA |
| | | EAHGTRPDLTDQPLPDADHTWYTNGSSLLQEG |
| | | QRKAGAAVTTETEVIWAKALPAGTSAQRAELI |
| | | ALTQALKMAEGKKLNVYTDSRYAFATAHIHGE |
| | | IYRRRGWLTSKGKEIKNKDEILALLKALFLPKR |
| | | LSIIHCPGHQKGHSAEARGNRMADQAARKAAI |
| | | TETPDTSTLL |
| 9 | T4-DNA- | ATGATCCTGAAGATTTTGAACGAGATCGCTAGC |
| | ligase_RNAseH- | ATCGGGAGTACAAAACAAAAGCAGGCGATCTT |
| | RT | AGAGAAAAACAAAGACAACGAGCTGTTGAAAC |
| | Nucleic Acid | GTGTGTATCGTTTAACCTATAGCCGTGGTCTTCA |
| | | GTACTACATCAAAAAGTGGCCGAAGCCAGGAA |
| | | TTGCCACACAATCTTTCGGCATGCTTACTTTAAC |
| | | TGACATGCTGGATTTCATCGAATTTACCCTGGC |
| | | AACGCGTAAATTGACCGGGAATGCTGCTATCGA |
| | | AGAGCTGACTGGTTATATTACCGACGGAAAAAA |
| | | AGACGACGTCGAAGTTTTGCGCCGCGTCATGAT |
| | | GCGTGATCTGGAATGCGGCGCGTCGGTCTCAAT |
| | | TGCTAACAAAGTCTGGCCTGGGCTGATTCCAGA |
| | | GCAGCCCCAAATGCTTGCAAGCAGTTACGATGA |
| | | GAAGGGTATTAACAAAAACATTAAGTTTCCTGC |
| | | ATTCGCTCAACTGAAGGCCGATGGTGCCCGCTG |
| | | TTTTGCGGAAGTCCGTGGCGACGAACTGGACGA |
| | | CGTGCGCCTTCTGTCTCGCGCTGGGAACGAATA |
| | | TTTGGGCTTGGATCTTCTTAAGGAAGAATTGAT |
| | | TAAGATGACCGCGGAGGCCCGTCAGATTCATCC |
| | | CGAAGGGGTGCTGATCGACGGCGAATTGGTATA |
| | | TCATGAGCAGGTAAAAAAAGAGCCCGAGGGAC |
| | | TGGACTTTCTTTTTGACGCATACCCGGAAAACT |
| | | CCAAGGCGAAGGAGTTCGCGGAGGTTGCTGAA |
| | | TCACGTACAGCCTCCAATGGGATCGCCAACAAA |
| | | TCGCTTAAGGGAACCATTTCCGAAAAGGAGGCA |
| | | CAGTGCATGAAATTCCAGGTCTGGGACTACGTC |
| | | CCCTTAGTAGAAATCTACTCCTTGCCTGCTTTCC |
| | | GTTTGAAATATGATGTCCGTTTCTCCAAATTGG |
| | | AGCAGATGACTTCTGGGTATGACAAGGTTATCT |
| | | TAATCGAAAATCAGGTCGTGAATAACCTGGACG |
| | | AAGCAAAAGTAATCTATAAAAAGTATATCGACC |
| | | AGGGATTAGAAGGCATTATTCTGAAAAACATCG |
| | | ACGGCTTATGGGAGAACGCGCGTTCGAAGAACT |
| | | TATATAAATTTAAGGAAGTTATCGATGTTGATC |
| | | TTAAGATCGTTGGGATCTACCCACATCGTAAAG |
| | | ATCCAACCAAGGCGGGAGGATTCATTTTAGAGT |
| | | CCGAATGCGGCAAGATTAAGGTAAACGCGGGT |
| | | TCTGGCCTTAAGGATAAGGCCGGGGTAAAGTCT |
| | | CACGAGTTGGATCGCACACGTATTATGGAAAAT |
| | | CAGAATTACTATATCGGCAAAATCTTAGAATGC |
| | | GAATGTAATGGGTGGTTGAAGAGCGATGGACG |
| | | CACCGATTATGTCAAATTATTTCTGCCGATCGCC |
| | | ATTCGCCTGCGCGAGGATAAAACGAAAGCGAA |
| | | CACTTTCGAGGATGTGTTCGGCGACTTTCACGA |
| | | AGTCACTGGCCTGGGTGGCAGCGGTGGCAGC |
| | | ATCCTGGCAGAGGCGCACGGCACTCGCCCG |
| | | GATCTGACGGACCAGCCGCTGCCGGACGCC |
| | | GATCATACCTGGTATACGAATGGTAGCAGCC |
| | | TGTTGCAAGAGGGTCAGCGTAAGGCCGGTGC |
| | | CGCGGTCACCACCGAGACTGAAGTGATTTGG |
| | | GCTAAAGCATTGCCTGCGGGTACCAGCGCGC |
| | | AGCGTGCCGAGCTGATCGCACTGACCCAAGC |
| | | GCTGAAAATGGCTGAGGGTAAGAAACTGAAT |
| | | GTGTACACGGATAGCCGTTATGCCTTTGCGA |
| | | CCGCCCACATTCACGGCGAGATCTATCGCCG |
| | | TCGCGGCTGGCTGACGTCCAAAGGCAAAGAG |
| | | ATCAAGAATAAAGACGAAATTCTGGCGCTGC |
| | | TGAAAGCGCTGTTCCTGCCGAAACGTCTGTC |
| | | GATCATCCATTGCCCGGGTCACCAGAAAGGC |
| | | CACAGCGCAGAGGCGCGTGGTAATCGCATG |
| | | GCTGACCAGGCTGCGCGTAAAGCCGCAATTA |
| | | CCGAAACCCCGGACACCAGCACGCTGCTGTA |
| | | A |
| 10 | T4-DNA- | MILKILNEIASIGSTKQKQAILEKNKDNELLKRVYR |
| | ligase_RNAseH- | LTYSRGLQYYIKKWPKPGIATQSFGMLTLTDMLD |
| | RT | FIEFTLATRKLTGNAAIEELTGYITDGKKDDVEVL |
| | Amino acid | RRVMMRDLECGASVSIANKVWPGLIPEQPQMLAS |
| | | SYDEKGINKNIKFPAFAQLKADGARCFAEVRGDE |
| | | LDDVRLLSRAGNEYLGLDLLKEELIKMTAEARQI |
| | | HPEGVLIDGELVYHEQVKKEPEGLDFLFDAYPENS |
| | | KAKEFAEVAESRTASNGIANKSLKGTISEKEAQC |
| | | MKFQVWDYVPLVEIYSLPAFRLKYDVRFSKLEQM |
| | | TSGYDKVILIENQVVNNLDEAKVIYKKYIDQGLEG |
| | | IILKNIDGLWENARSKNLYKFKEVIDVDLKIVGIYP |
| | | HRKDPTKAGGFILESECGKIKVNAGSGLKDKAGV |
| | | KSHELDRTRIMENQNYYIGKILECECNGWLKSDG |
| | | RTDYVKLFLPIAIRLREDKTKANTFEDVFGDFHEV |
| | | TGLGGSGGSILAEAHGTRPDLTDQPLPDADHT |
| | | WYTNGSSLLQEGQRKAGAAVTTETEVIWAKA |
| | | LPAGTSAQRAELIALTQALKMAEGKKLNVYTD |
| | | SRYAFATAHIHGEIYRRRGWLTSKGKEIKNKD |
| | | EILALLKALFLPKRLSIIHCPGHQKGHSAEARG |
| | | NRMADQAARKAAITETPDTSTLL |
| 11 | T4-RNA- | ATGTTTAAAAAGTATAGTAGCCTGGAAAATCAC |
| | ligase2_RNAse | TATAATAGCAAATTTATTGAGAAATTGTACTCA |
| | H-RT | TTAGGTCTTACCGGAGGAGAGTGGGTAGCGCGT |
| | Nucleic acid | GAGAAAATCCATGGGACTAACTTTTCACTTATT |
| | | ATCGAGCGTGATAAGGTGACTTGCGCTAAACGC |
| | | ACTGGGCCCATCCTGCCCGCAGAAGACTTCTTC |
| | | GGTTATGAGATTATTCTGAAAAACTATGCCGAC |
| | | TCGATCAAGGCAGTTCAAGACATCATGGAAACG |
| | | TCGGCAGTAGTCTCCTACCAGGTATTTGGCGAA |
| | | TTCGCAGGTCCTGGCATTCAGAAGAATGTAGAC |
| | | TACTGTGATAAAGATTTCTACGTCTTCGACATC |
| | | ATTGTGACCACTGAGTCGGGAGATGTTACCTAT |
| | | GTAGACGATTATATGATGGAGAGTTTCTGTAAC |
| | | ACCTTCAAATTTAAGATGGCACCGTTATTAGGT |
| | | CGCGGTAAATTTGAGGAGCTGATTAAGTTACCA |
| | | AATGACTTAGACAGCGTTGTACAGGATTACAAT |
| | | TTCACAGTCGATCATGCCGGTTTAGTTGACGCT |
| | | AACAAATGTGTATGGAACGCTGAGGCAAAAGG |
| | | CGAAGTGTTTACCGCCGAAGGTTATGTTCTGAA |
| | | ACCTTGTTATCCAAGCTGGTTGCGTAACGGCAA |
| | | CCGTGTCGCGATCAAATGCAAGAACTCTAAATT |
| | | CAGTGAAAAGAAAAAAAGCGATAAGCCGATTA |
| | | AGGCTAAGGTAGAGTTATCCGAGGCTGACAATA |
| | | AGTTAGTGGGAATCTTGGCGTGTTATGTGACGC |
| | | TGAATCGCGTGAACAATGTTATCTCTAAAATTG |
| | | GAGAAATCGGCCCAAAGGACTTTGGAAAGGTC |
| | | ATGGGACTTACGGTCCAGGATATTCTGGAGGAA |
| | | ACGTCTCGCGAGGGTATTACCTTAACGCAGGCA |
| | | GACAATCCGTCCCTTATCAAAAAAGAATTGGTC |
| | | AAAATGGTCCAGGACGTGCTGCGCCCTGCGTGG |
| | | ATTGAGCTTGTGAGCGGTGGCAGCGGTGGCA |
| | | GCATCCTGGCAGAGGCGCACGGCACTCGCCC |
| | | GGATCTGACGGACCAGCCGCTGCCGGACGC |
| | | CGATCATACCTGGTATACGAATGGTAGCAGC |
| | | CTGTTGCAAGAGGGTCAGCGTAAGGCCGGTG |
| | | CCGCGGTCACCACCGAGACTGAAGTGATTTG |
| | | GGCTAAAGCATTGCCTGCGGGTACCAGCGCG |
| | | CAGCGTGCCGAGCTGATCGCACTGACCCAAG |
| | | CGCTGAAAATGGCTGAGGGTAAGAAACTGAA |
| | | TGTGTACACGGATAGCCGTTATGCCTTTGCG |
| | | ACCGCCCACATTCACGGCGAGATCTATCGCC |
| | | GTCGCGGCTGGCTGACGTCCAAAGGCAAAGA |
| | | GATCAAGAATAAAGACGAAATTCTGGCGCTG |
| | | CTGAAAGCGCTGTTCCTGCCGAAACGTCTGT |
| | | CGATCATCCATTGCCCGGGTCACCAGAAAGG |
| | | CCACAGCGCAGAGGCGCGTGGTAATCGCATG |
| | | GCTGACCAGGCTGCGCGTAAAGCCGCAATTA |
| | | CCGAAACCCCGGACACCAGCACGCTGCTGTA |
| | | A |
| 12 | T4-RNA- | MFKKYSSLENHYNSKFIEKLYSLGLTGGEWVARE |
| | ligase2_RNAse | KIHGTNFSLIIERDKVTCAKRTGPILPAEDFFGYEII |
| | H-RT | LKNYADSIKAVQDIMETSAVVSYQVFGEFAGPGI |
| | Amino Acid | QKNVDYCDKDFYVFDIIVTTESGDVTYVDDYMM |
| | | ESFCNTFKFKMAPLLGRGKFEELIKLPNDLDSVVQ |
| | | DYNFTVDHAGLVDANKCVWNAEAKGEVFTAEG |
| | | YVLKPCYPSWLRNGNRVAIKCKNSKFSEKKKSDK |
| | | PIKAKVELSEADNKLVGILACYVTLNRVNNVISKI |
| | | GEIGPKDFGKVMGLTVQDILEETSREGITLTQADN |
| | | PSLIKKELVKMVQDVLRPAWIELVSGGSGGSILA |
| | | EAHGTRPDLTDQPLPDADHTWYTNGSSLLQEG |
| | | QRKAGAAVTTETEVIWAKALPAGTSAQRAELI |
| | | ALTQALKMAEGKKLNVYTDSRYAFATAHIHGE |
| | | IYRRRGWLTSKGKEIKNKDEILALLKALFLPKR |
| | | LSIIHCPGHQKGHSAEARGNRMADQAARKAAI |
| | | TETPDTSTLL |
| 13 | RT-RNAseH | ILAEAHGTRPDLTDQPLPDADHTWYTNGSSLLQ |
| | domain | EGQRKAGAAVTTETEVIWAKALPAGTSAQRAE |
| | | LIALTQALKMAEGKKLNVYTDSRYAFATAHIH |
| | | GEIYRRRGWLTSKGKEIKNKDEILALLKALFLP |
| | | KRLSIIHCPGHQKGHSAEARGNRMADQAARKA |
| | | AITETPDTSTLL |
| 14 | Engineered | MTWLSDFPQAWAETGGMGLAVRQAPLIIPLKATS |
| | reverse | TPVSIKQYPMSQKARLGIKPHIQRLLDQGILVPCQS |
| | transcriptase | PWNTPLLPVKKPGTNDYRPVQDLREVNKRVEDIH |
| | (MMLV RT | PTVPNPYNLLSGPPPSHQWYTVLDLKDAFFCLRL |
| | Variant) | HPTSQPLFAFEWRDPEMGISGQLTWTRLPQGFKN |
| | | SPTLFNEALHRDLADFRIQHPDLILLQYVDDLLLA |
| | | ATSELDCQQGTRALLQTLGNLGYRASAKKAQICQ |
| | | KQVKYLGYLLKEGQRWLTEARKETVMGQPTPKT |
| | | PRQLRRFLGKAGFCRLFIPGFAEMAAPLYPLTKPG |
| | | TLFNWGPDQQKAYQEIKQALLTAPALGLPDLTKP |
| | | FELFVDEKQGYAKGVLTQKLGPWRRPVAYLSKK |
| | | LDPVAAGWPPCLRMVAAIAVLTKDAGKLTMGQP |
| | | LVIGAPHAVEALVKQPAGRWLSKARMTHYQALL |
| | | LDTDRVQFGPVVALNPATLLPLPEEGLQHNCLDIL |
| | | AEAHGTRPDLTDQPLPDADHTWYTNGSSLLQEGQ |
| | | RKAGAAVTTETEVIWAKALPAGTSAQRAELIALT |
| | | QALKMAEGKKLNVYTDSRYAFATAHIHGEIYRRR |
| | | GWLTSKGKEIKNKDEILALLKALFLPKRLSIIHCPG |
| | | HQKGHSAEARGNRMADQAARKAAITETPDTSTLL |
| | | IENSSPNSRLIN |
| 15 | Wild-type | TLNIEDEHRLHETSKEPDVSLGSTWLSDFPQAWAE |
| | MMLV | TGGMGLAVRQAPLIIPLKATSTPVSIKQYPMSQEA |
| | GenBank Seq ID | RLGIKPHIQRLLDQGILVPCQSPWNTPLLPVKKPGT |
| | NP_955591.1 | NDYRPVQDLREVNKRVEDIHPTVPNPYNLLSGLPP |
| | p80 RT | SHQWYTVLDLKDAFFCLRLHPTSQPLFAFEWRDPE |
| | | MGISGQLTWTRLPQGFKNSPTLFDEALHRDLADFR |
| | | IQHPDLILLQYVDDLLLAATSELDCQQGTRALLQT |
| | | LGNLGYRASAKKAQICQKQVKYLGYLLKEGQRW |
| | | LTEARKETVMGQPTPKTPRQLREFLGTAGFCRLWI |
| | | PGFAEMAAPLYPLTKTGTLFNWGPDQQKAYQEIK |
| | | QALLTAPALGLPDLTKPFELFVDEKQGYAKGVLT |
| | | QKLGPWRRPVAYLSKKLDPVAAGWPPCLRMVAA |
| | | IAVLTKDAGKLTMGQPLVILAPHAVEALVKQPPDR |
| | | WLSNARMTHYQALLLDTDRVQFGPVVALNPATLL |
| | | PLPEEGLQHNCLDILAEAHGTRPDLTDQPLPDADH |
| | | TWYTDGSSLLQEGQRKAGAAVTTETEVIWAKALP |
| | | AGTSAQRAELIALTQALKMAEGKKLNVYTDSRYA |
| | | FATAHIHGEIYRRRGLLTSEGKEIKNKDEILALLKA |
| | | LFLPKRLSIIHCPGHQKGHSAEARGNRMADQAAR |
| | | KAAITETPDTSTLL |
| 16 | Linker | GGTGGCAGCGGTGGCAGC |
| | Nucleic acid | |
| 17 | Linker | GGSGGS |
| 18 | Linker | GGGGS |
| 19 | Linker | GSGGSG |
| 20 | Linker | GGTTCAGGGGGTTCCGGT |
| 21 | Tag | HHHHHH |
| 22 | endoprotein | ENLYFQ/G |
| | cleavage site | |
| 23 | endoprotein | DDDDK/ |
| | cleavage site | |
| 24 | endoprotein | IEGR/ |
| | cleavage site | |
| 25 | endoprotein | LVPR/GS |
| | cleavage site | |
| 26 | endoprotein | LEVLFQ/GP |
| | cleavage site | |
| 27 | Tag | SEEDEEKEEDG |
| 28 | LigAT | ATGGCTATTCAGAAGCCGTTACTGGCGGCCAGC |
| | (Acanthocystis | CTGAAGAAAATGTCTGTTGACAACTTGACTTTCC |
| | turfacea Ligase | CGGTCTACGCAACGCCGAAACTGGACGGTATCC |
| | 1; ATCV-1) | GTGCGCTGAAGATTGACGGCACCCTGGTGTCGC |
| | Nucleic acid | GTACGTTCAAACCGATCCGCAATACTACCATCTC |
| | | CAAGGTCCTGGCGAGCTTGCTGCCGGACGGCAG |
| | | CGATGGTGAGATCTTGTCTGGTAAGACCTTTCAG |
| | | GATAGCACCAGCACCGTGATGACGACCGATGCG |
| | | GGTATCGGTAGCGATACCACCTTCTTCTGGTTTG |
| | | ATTACGTTAAAGACGACCCGGATAAGGGTTACC |
| | | TGGACCGTATCGCTGACATGAAAACCTTCGTCG |
| | | ACCAGCACCCGGAAATCCTGAAAGACAGCTGCG |
| | | TGACGATCGTCCCGCTGTTCCCGAAGAAAATTG |
| | | ACACGCCAGAAGAACTGCATGTGTTTGAGAAAT |
| | | GGTGTCTGGACCAGGGTTTTGAGGGCGTTATGG |
| | | TGCGTACGGCCGGTGGCAAGTATAAGTTTGGTC |
| | | GCAGCACCGAGAAAGAACAAATCCTGGTTAAGA |
| | | TTAAACAGTTTGAAGATGATGAAGCTGTTGTGA |
| | | TCGGTGTGAGCGCGCTGCAAACCAATACGAACG |
| | | ATAAGAAACTGAATCAGCTGGGTGAGATGCGTC |
| | | GTACTAGCCACCAAGACGGCAAAGTTGACCTGG |
| | | ACATGCTGGGTGCGCTTGACGTGGACTGGAATG |
| | | GTATTCGTTTCAGCATCGGTACGGGCTTTGATAA |
| | | AGACACCCGCGAAGATCTCTGGAAACAACGCGA |
| | | CAGCATCGTGGGTAAAGTCGTTAAGTTCAAGTA |
| | | TTTCTCCCAGGGTATTAAGACCGCTCCGCGTTTT |
| | | CCTGTGTTTCTGGGTTTTCGTGACGAGAACGATA |
| | | TG |
| 29 | LigAT | MAIQKPLLAASLKKMSVDNLTFPVYATPKLDGIRA |
| | (Acanthocystis | LKIDGTLVSRTFKPIRNTTISKVLASLLPDGSDGEIL |
| | turfacea Ligase | SGKTFQDSTSTVMTTDAGIGSDTTFFWFDYVKDDP |
| | 1; ATCV-1) | DKGYLDRIADMKTFVDQHPEILKDSCVTIVPLFPK |
| | Amino acid | KIDTPEELHVFEKWCLDQGFEGVMVRTAGGKYKF |
| | | GRSTEKEQILVKIKQFEDDEAVVIGVSALQTNTND |
| | | KKLNQLGEMRRTSHQDGKVDLDMLGALDVDWN |
| | | GIRFSIGTGFDKDTREDLWKQRDSIVGKVVKFKYF |
| | | SQGIKTAPRFPVFLGFRDENDM |
| 30 | E. Coli | ATGCTGAAGCAGGTGGAAATCTTTACAAACG |
| | Ribonuclease H | GTTCTTGTCTGGGAAATCCTGGACCCGGAGG |
| | (RNAseH) | ATATGGTGCAATTTTGCGTTATCGCGGACGC |
| | Nucleic Acid | GAAAAGACATTTTCAGCGGGATATACTCGTAC |
| | | AACGAACAACCGCATGGAACTTATGGCCGCA |
| | | ATCGTGGCACTTGAGGCGCTGAAGGAACATT |
| | | GCGAAGTGATCCTGTCTACAGATAGTCAATAT |
| | | GTGCGCCAAGGAATTACGCAGTGGATCCATA |
| | | ATTGGAAGAAGCGTGGCTGGAAGACCGCAGA |
| | | CAAAAAACCAGTGAAAAACGTAGACTTATGG |
| | | CAACGTCTGGATGCTGCTCTGGGACAACACC |
| | | AAATCAAATGGGAATGGGTGAAGGGACATGC |
| | | CGGACATCCCGAGAATGAGCGTTGTGACGAA |
| | | TTAGCGCGTGCCGCCGCAATGAATCCTACGC |
| | | TGGAAGACACCGGTTATCAGGTGGAGGTTTA |
| | | A |
| 31 | E. Coli | MLKQVEIFTNGSCLGNPGPGGYGAILRYRGREKTF |
| | Ribonuclease H | SAGYTRTTNNRMELMAAIVALEALKEHCEVILSTD |
| | (RNAseH) | SQYVRQGITQWIHNWKKRGWKTADKKPVKNVDL |
| | Amino Acid | WQRLDAALGQHQIKWEWVKGHAGHPENERCDEL |
| | | ARAAAMNPTLEDTGYQVEV |
| 32 | inactive | ATGATCATTGGTTACTTCAACGGGCTTTGCGA |
| | RNAseHI from | ACCTAAAAACCCCGGCGGTATTGCGACCTTT |
| | Sulfobolus | GGCTTCGTCATTTACCTGGATAACCGCAAAAT |
| | tokodaii | CGAGGGCTATGGTTTAGCCGAGAAGCCATTC |
| | (RNAseHI-Sto) | TCTATCAACTCCACCAATAATGTTGCCGAGTA |
| | | TTCCGGTTTGATCTGCCTTATGGAGACAATGT |
| | | TACGCTTAGGAATTAGTAGTCCCATTATTAAA |
| | | GGTGACTCACAATTGGTTATCAAACAGATGAA |
| | | TGGCGAGTACAAGGTCAAAGCGAAACGTATT |
| | | ATCCCTTTATACGAGAAAGCTATTGAATTGAA |
| | | AAAGAAGTTGAATGCCACCCTTATTTGGGTGC |
| | | CTCGCGAGGAGAATAAGGAGGCTGATCGTTT |
| | | GTCTCGTGTCGCCTATGAGTTGGTGCGTCGC |
| | | GGCAAATTGCGTGACATTGGGTGCATTATTCT |
| | | GACGTAA |
| 33 | Inactive | MIIGYFNGLCEPKNPGGIATFGFVIYLDNRKIEG |
| | RNAseHI from | YGLAEKPFSINSTNNVAEYSGLICLMETMLRLGI |
| | Sulfobolus | SSPIIKGDSQLVIKQMNGEYKVKAKRIIPLYEKA |
| | tokodaii | IELKKKLNATLIWVPREENKEADRLSRVAYELV |
| | (RNAseHI-Sto) | RRGKLRDIGCIILT |
| 34 | MMLV-RT | ATCCTGGCAGAGGCGCACGGCACTCGCCCGG |
| | variant RNAseH | ATCTGACGGACCAGCCGCTGCCGGACGCCGA |
| | domain | TCATACCTGGTATACGAATGGTAGCAGCCTGT |
| | | TGCAAGAGGGTCAGCGTAAGGCCGGTGCCGC |
| | | GGTCACCACCGAGACTGAAGTGATTTGGGCT |
| | | AAAGCATTGCCTGCGGGTACCAGCGCGCAGC |
| | | GTGCCGAGCTGATCGCACTGACCCAAGCGCT |
| | | GAAAATGGCTGAGGGTAAGAAACTGAATGTG |
| | | TACACGGATAGCCGTTATGCCTTTGCGACCG |
| | | CCCACATTCACGGCGAGATCTATCGCCGTCG |
| | | CGGCTGGCTGACGTCCAAAGGCAAAGAGATC |
| | | AAGAATAAAGACGAAATTCTGGCGCTGCTGA |
| | | AAGCGCTGTTCCTGCCGAAACGTCTGTCGAT |
| | | CATCCATTGCCCGGGTCACCAGAAAGGCCAC |
| | | AGCGCAGAGGCGCGTGGTAATCGCATGGCTG |
| | | ACCAGGCTGCGCGTAAAGCCGCAATTACCGA |
| | | AACCCCGGACACCAGCACGCTGCTGTAA |
| 35 | T4 DNA ligase | ATGATCCTGAAGATTTTGAACGAGATCGCTAG |
| | | CATCGGGAGTACAAAACAAAAGCAGGCGATC |
| | | TTAGAGAAAAACAAAGACAACGAGCTGTTGA |
| | | AACGTGTGTATCGTTTAACCTATAGCCGTGGT |
| | | CTTCAGTACTACATCAAAAAGTGGCCGAAGC |
| | | CAGGAATTGCCACACAATCTTTCGGCATGCTT |
| | | ACTTTAACTGACATGCTGGATTTCATCGAATT |
| | | TACCCTGGCAACGCGTAAATTGACCGGGAAT |
| | | GCTGCTATCGAAGAGCTGACTGGTTATATTAC |
| | | CGACGGAAAAAAAGACGACGTCGAAGTTTTG |
| | | CGCCGCGTCATGATGCGTGATCTGGAATGCG |
| | | GCGCGTCGGTCTCAATTGCTAACAAAGTCTG |
| | | GCCTGGGCTGATTCCAGAGCAGCCCCAAATG |
| | | CTTGCAAGCAGTTACGATGAGAAGGGTATTA |
| | | ACAAAAACATTAAGTTTCCTGCATTCGCTCAA |
| | | CTGAAGGCCGATGGTGCCCGCTGTTTTGCGG |
| | | AAGTCCGTGGCGACGAACTGGACGACGTGCG |
| | | CCTTCTGTCTCGCGCTGGGAACGAATATTTGG |
| | | GCTTGGATCTTCTTAAGGAAGAATTGATTAAG |
| | | ATGACCGCGGAGGCCCGTCAGATTCATCCCG |
| | | AAGGGGTGCTGATCGACGGCGAATTGGTATA |
| | | TCATGAGCAGGTAAAAAAAGAGCCCGAGGGA |
| | | CTGGACTTTCTTTTTGACGCATACCCGGAAAA |
| | | CTCCAAGGCGAAGGAGTTCGCGGAGGTTGCT |
| | | GAATCACGTACAGCCTCCAATGGGATCGCCA |
| | | ACAAATCGCTTAAGGGAACCATTTCCGAAAA |
| | | GGAGGCACAGTGCATGAAATTCCAGGTCTGG |
| | | GACTACGTCCCCTTAGTAGAAATCTACTCCTT |
| | | GCCTGCTTTCCGTTTGAAATATGATGTCCGTT |
| | | TCTCCAAATTGGAGCAGATGACTTCTGGGTAT |
| | | GACAAGGTTATCTTAATCGAAAATCAGGTCGT |
| | | GAATAACCTGGACGAAGCAAAAGTAATCTAT |
| | | AAAAAGTATATCGACCAGGGATTAGAAGGCA |
| | | TTATTCTGAAAAACATCGACGGCTTATGGGAG |
| | | AACGCGCGTTCGAAGAACTTATATAAATTTAA |
| | | GGAAGTTATCGATGTTGATCTTAAGATCGTTG |
| | | GGATCTACCCACATCGTAAAGATCCAACCAA |
| | | GGCGGGAGGATTCATTTTAGAGTCCGAATGC |
| | | GGCAAGATTAAGGTAAACGCGGGTTCTGGCC |
| | | TTAAGGATAAGGCCGGGGTAAAGTCTCACGA |
| | | GTTGGATCGCACACGTATTATGGAAAATCAG |
| | | AATTACTATATCGGCAAAATCTTAGAATGCGA |
| | | ATGTAATGGGTGGTTGAAGAGCGATGGACGC |
| | | ACCGATTATGTCAAATTATTTCTGCCGATCGC |
| | | CATTCGCCTGCGCGAGGATAAAACGAAAGCG |
| | | AACACTTTCGAGGATGTGTTCGGCGACTTTCA |
| | | CGAAGTCACTGGCCTG |
| 36 | T4 DNA ligase | MILKILNEIASIGSTKQKQAILEKNKDNELLKRV |
| | | YRLTYSRGLQYYIKKWPKPGIATQSFGMLTLTD |
| | | MLDFIEFTLATRKLTGNAAIEELTGYITDGKKD |
| | | DVEVLRRVMMRDLECGASVSIANKVWPGLIPE |
| | | QPQMLASSYDEKGINKNIKFPAFAQLKADGARC |
| | | FAEVRGDELDDVRLLSRAGNEYLGLDLLKEELI |
| | | KMTAEARQIHPEGVLIDGELVYHEQVKKEPEG |
| | | LDFLFDAYPENSKAKEFAEVAESRTASNGIANKS |
| | | LKGTISEKEAQCMKFQVWDYVPLVEIYSLPAFR |
| | | LKYDVRFSKLEQMTSGYDKVILIENQVVNNLDE |
| | | AKVIYKKYIDQGLEGIILKNIDGLWENARSKNL |
| | | YKFKEVIDVDLKIVGIYPHRKDPTKAGGFILESE |
| | | CGKIKVNAGSGLKDKAGVKSHELDRTRIMENQ |
| | | NYYIGKILECECNGWLKSDGRTDYVKLFLPIAIR |
| | | LREDKTKANTFEDVFGDFHEVTGL |
| 37 | T4 RNA ligase2 | ATGTTTAAAAAGTATAGTAGCCTGGAAAATCAC |
| | | TATAATAGCAAATTTATTGAGAAATTGTACTCAT |
| | | TAGGTCTTACCGGAGGAGAGTGGGTAGCGCGTG |
| | | AGAAAATCCATGGGACTAACTTTTCACTTATTAT |
| | | CGAGCGTGATAAGGTGACTTGCGCTAAACGCAC |
| | | TGGGCCCATCCTGCCCGCAGAAGACTTCTTCGGT |
| | | TATGAGATTATTCTGAAAAACTATGCCGACTCG |
| | | ATCAAGGCAGTTCAAGACATCATGGAAACGTCG |
| | | GCAGTAGTCTCCTACCAGGTATTTGGCGAATTCG |
| | | CAGGTCCTGGCATTCAGAAGAATGTAGACTACT |
| | | GTGATAAAGATTTCTACGTCTTCGACATCATTGT |
| | | GACCACTGAGTCGGGAGATGTTACCTATGTAGA |
| | | CGATTATATGATGGAGAGTTTCTGTAACACCTTC |
| | | AAATTTAAGATGGCACCGTTATTAGGTCGCGGT |
| | | AAATTTGAGGAGCTGATTAAGTTACCAAATGAC |
| | | TTAGACAGCGTTGTACAGGATTACAATTTCACA |
| | | GTCGATCATGCCGGTTTAGTTGACGCTAACAAAT |
| | | GTGTATGGAACGCTGAGGCAAAAGGCGAAGTGT |
| | | TTACCGCCGAAGGTTATGTTCTGAAACCTTGTTA |
| | | TCCAAGCTGGTTGCGTAACGGCAACCGTGTCGC |
| | | GATCAAATGCAAGAACTCTAAATTCAGTGAAAA |
| | | GAAAAAAAGCGATAAGCCGATTAAGGCTAAGGT |
| | | AGAGTTATCCGAGGCTGACAATAAGTTAGTGGG |
| | | AATCTTGGCGTGTTATGTGACGCTGAATCGCGTG |
| | | AACAATGTTATCTCTAAAATTGGAGAAATCGGC |
| | | CCAAAGGACTTTGGAAAGGTCATGGGACTTACG |
| | | GTCCAGGATATTCTGGAGGAAACGTCTCGCGAG |
| | | GGTATTACCTTAACGCAGGCAGACAATCCGTCC |
| | | CTTATCAAAAAAGAATTGGTCAAAATGGTCCAG |
| | | GACGTGCTGCGCCCTGCGTGGATTGAGCTTGTG |
| | | AGC |
| 38 | T4 RNA ligase2 | MFKKYSSLENHYNSKFIEKLYSLGLTGGEWVARE |
| | | KIHGTNFSLIIERDKVTCAKRTGPILPAEDFFGYEIIL |
| | | KNYADSIKAVQDIMETSAVVSYQVFGEFAGPGIQK |
| | | NVDYCDKDFYVFDIIVTTESGDVTYVDDYMMESF |
| | | CNTFKFKMAPLLGRGKFEELIKLPNDLDSVVQDYN |
| | | FTVDHAGLVDANKCVWNAEAKGEVFTAEGYVLK |
| | | PCYPSWLRNGNRVAIKCKNSKFSEKKKSDKPIKAK |
| | | VELSEADNKLVGILACYVTLNRVNNVISKIGEIGPK |
| | | DFGKVMGLTVQDILEETSREGITLTQADNPSLIKKE |
| | | LVKMVQDVLRPAWIELVS |
| 39 | PBCV-1 Ligase | MAITKPLLAATLENIEDVQFPCLATPKIDGIRSVKQ |
| | | TQMLSRTFKPIRNSVMNRLLTELLPEGSDGEISIEG |
| | | ATFQDTTSAVMTGHKMYNAKFSYYWFDYVTDDP |
| | | LKKYIDRVEDMKNYITVHPHILEHAQVKIIPLIPVEI |
| | | NNITELLQYERDVLSKGFEGVMIRKPDGKYKFGRS |
| | | TLKEGILLKMKQFKDAEATIISMTALFKNTNTKTK |
| | | DNFGYSKRSTHKSGKVEEDVMGSIEVDYDGVVFSI |
| | | GTGFDADQRRDFWQNKESYIGKMVKFKYFEMGS |
| | | KDCPRFPVFIGIRHEEDR |
| 40 | LigAT-HU | MAIQKPLLAASLKKMSVDNLTFPVYATPKLDGIRA |
| | | LKIDGTLVSRTFKPIRNTTISKVLASLLPDGSDGEIL |
| | | SGKTFQDSTSTVMTTDAGIGSDTTFFWFDYVKDDP |
| | | DKGYLDRIADMKTFVDQHPEILKDSCVTIVPLFPK |
| | | KIDTPEELHVFEKWCLDQGFEGVMVRTAGGKYKF |
| | | GRSTEKEQILVKIKQFEDDEAVVIGVSALQTNTND |
| | | KKLNQLGEMRRTSHQDGKVDLDMLGALDVDWN |
| | | GIRFSIGTGFDKDTREDLWKQRDSIVGKVVKFKYF |
| | | SQGIKTAPRFPVFLGFRDENDMGGSGGSMNKTQLI |
| | | DVIAEKAELSKTQAKAALESTLAAITESLKEGDAV |
| | | QLVGFGTFKVNHRAERTGRNPQTGKEIKIAAANVP |
| | | AFVSGKALKDAVK |
| 41 | Template for | rGrArGrCrArGrUrUrArArGrCrCrArGrArGrCrCrArArC |
| | row 'AA | rUrUrArCrUrCrCrUrCrUrGrUrGrGrArArGrUrCrUrCr |
| | | UrCrC |
| 42 | Substrate (A) | GAG ACT TCC ACA GAG GAG TA |
| 43 | Substrate (pA) | /5Phos/AGT TGG CTC TGG CTT AAC TGC GCT |
| | | AAC TTC-dye |

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Designs of Novel Chimeric Ligase Enzymes

This example demonstrates the design and generation of chimeric ligase polypeptides described herein. The chimeric ligase polypeptides were engineered by fusing a ligase to a nucleic acid binding domain that is specific for RNA:DNA hybrid duplex.

Numerous chimeric ligase enzymes, comprising a ligase enzyme operably linked to a nucleic acid binding domain that is specific for RNA:DNA hybrid duplex, were designed and engineered to produce chimeric ligase enzymes with improved RNA templated DNA ligation activity. RNAseH was initially selected because it comprises a nucleic acid binding domain that is capable of selectively binding RNA-DNA hybrid duplex even in the presence of excess of double stranded DNA. However, all enzymes that act on RNA-DNA hybrid duplexes can be used to engineer the chimeric ligase. Specifically, all enzymes that contain an RNAseH domain can be used. For example, reverse transcriptase enzymes have an RNAseH domain that can be used. However, the RNAseH catalytic domain was inactivated with a mutation that renders the RNAseH catalytically inactive (e.g., DION mutation in E. coli RNAseH or D524N in MMLV RT RNAseH). Exemplary chimeric ligase polypeptides (or chimeric ligase enzymes) are described below.

LigAT_C-RNAseH-Ecoli. A chimeric ligase polypeptide comprising a LigAT ligase fused to a catalytically inactive RNAseH from E. coli at the C-terminus.

A chimeric enzyme comprising the amino acid sequence of SEQ ID NO: 2 or encoded by the nucleic acid sequence of SEQ ID NO: 1 was successfully purified. These ligases were tested using in vitro assays (e.g., capillary electrophoresis) and in an in situ workflow assay. The chimeric ligase enzyme comprising the amino acid sequence of SEQ ID NO: 2 was demonstrated to be an active ligase for RNA-templated DNA ligation. In addition, the chimeric ligase enzyme had improved properties when compared to a wild-type ligase (LigAT) or a control recombinant ligase. The control ligase comprised LigAT fused to single stranded DNA binding protein (HU; LigAT-HU). Specifically, the chimeric ligase enzyme LigAT_C-RNAseH-Ecoli showed decreased ligation junction bias when compared to LigAT-HU. LigAT_C-RNAseH-Ecoli also showed improved thermostability when compared to LigAT or LigAT-HU.

LigAT_N-RNAseH-Ecoli. A chimeric ligase polypeptide comprising a LigAT ligase fused to a catalytically inactive RNAseH from E. coli at the N-terminus. The chimeric ligase enzyme comprising the amino acid sequence of SEQ ID NO: 4 or encoded by the nucleic acid sequence of SEQ ID NO: 3 was successfully purified. The expression of LigAT_N-RNAseH-Ecoli was weak when compared to the expression of LigAT_C-RNAseH-Ecoli.

LigAT_C-RNAseH-Sto. A chimeric ligase polypeptide comprising a LigAT ligase fused to a catalytically inactive RNAseH from Sulfobolus tokodaii (sto; S. tokodai) at the C-terminus. The chimeric ligase enzyme, which comprises the amino acid sequence of SEQ ID NO: 6 or is encoded by the nucleic acid sequence of SEQ ID NO: 5, was successfully purified and evaluated. In vitro assays (e.g., capillary electrophoresis; CE) and the in situ workflow assay demonstrated that the LigAT_C-RNAseH-Sto chimeric ligase was an active ligase for RNA-templated DNA ligation. In addition, the chimeric ligase enzyme had improved properties when compared to a wild-type ligase (LigAT) or a recombinant ligase comprising LigAT fused to single stranded DNA binding protein (HU; LigAT-HU; SEQ ID NO: 40). Specifically, LigAT_C-RNAseH-Sto showed improved thermostability when compared to LigAT or LigAT-HU.

LigAT_C-RNAseH-RT. A chimeric ligase enzyme comprising LigAT ligase fused to a catalytically inactive RNAseH domain from a reverse transcriptase (RT) enzyme. The RT is a MMLV variant (FIG. 12; Table 3). The chimeric ligase enzyme, which comprises the amino acid sequence of SEQ ID NO: 8 or is encoded by the nucleic acid sequence of SEQ ID NO: 7, was successfully purified and evaluated. In vitro assays (e.g., capillary electrophoresis) and an in situ workflow assay demonstrated that the LigAT_C-RNAseH-RT chimeric ligase was an active ligase for RNA-templated DNA ligation.

T4-DNA-ligase_RNAseH-RT. A chimeric ligase enzyme comprising a T4 DNA ligase operably linked to a catalytically inactive RNAseH domain from a reverse transcriptase (RT) enzyme. This is an example of a different ligase (not LigAT) fused to catalytically inactive RNAseH. The chimeric ligase enzyme, which comprises the amino acid sequence of SEQ ID NO: 10 or is encoded by the nucleic acid sequence of SEQ ID NO: 9, was successfully expressed. In vitro assays (e.g., capillary electrophoresis) and an in situ workflow assay demonstrated that the chimeric ligase of SEQ ID NO: 10 was an active ligase for RNA-templated DNA ligation. However, this chimeric ligase showed reduced levels of RNA templated DNA ligation when compared to any one of the other fusions including an RNAseH domain. The observed low levels of RNA templated DNA ligation were consistent with the activity of the wild-type T4 DNA ligase, which has been reported in the literature.

T4-RNA-ligase2_RNAseH-RT. A chimeric ligase enzyme comprising a T4 RNA ligase2 operably linked to a catalytically inactive RNAseH domain from a reverse transcriptase (RT) enzyme.

This is another example of a different ligase (not LigAT) fused to a catalytically inactive RNAseH domain. The chimeric ligase enzyme, which comprises the amino acid sequence of SEQ ID NO: 12 or is encoded by the nucleic acid sequence of SEQ ID NO: 11 was successfully expressed. In vitro assays (e.g., capillary electrophoresis) and an in situ workflow assay demonstrated that the T4-RNA-ligase2_RNAseH-RT chimeric ligase was an active ligase for RNA-templated DNA ligation. However, the T4-RNA-ligase2_RNAseH-RT chimeric ligase showed reduced levels of RNA templated DNA ligation activity when compared to any one of T4-DNA-ligase_RNAseH-RT, LigAT_C-RNAseH-RT, LigAT_C-RNAseH-Sto, LigAT_N-RNAseH-Ecoli, or LigAT_C-RNAseH-Ecoli. The levels of RNA templated DNA ligation activity were higher than the levels observed with the T4-DNA-ligase_RNAseH-RT chimeric enzyme. The observed reduced levels of RNA templated DNA ligation were also consistent with the reported activity of the wild-type T4 RNA ligase2.

To generate these chimeric ligases, ligase genes were inserted into a pD451 plasmid (ATUM) or a pJ401 plasmid (ATUM) and expressed in BL21 DE3 gold (Agilent) or XL1 Blue MR (Agilent).

### Example 2: Purification of LigAT_c-RNAseH-Ecoli Procedure & Yield Summary

The chimeric ligase enzymes disclosed herein were purified using methods known in the art. The nucleic acids encoding each enzyme (LigAT, or LigAT_c-RNAseH-Ecoli) were individually expressed into a pD451_N-His-SUMO vector. All enzymes were expressed in E. coli BL21 DE3 gold and cultured in 500 mL LB. The cells were induced with 0.6 mM IPTG and were cultured overnight at 18° C. to induce protein expression. Cells were centrifuged and the cell pellets were lysed using a microfluidizer. The lysates were treated with 0.5% PEI to precipitate nucleic acids. Ammonium sulfate was added to the lysates to remove residual PEI. The lysates were then run through Nickel column #1 (AKTA FPLC) and the eluents were precipitated with ammonium. The ammonium precipitated nickel column protein-containing eluents were treated with Ulp1 protease overnight at 4° C. This was followed by Nickel column #2 (AKTA FPLC) purification to remove Ulp1, the cleavage product, and remaining SUMO-containing protein.

The purified chimeric ligases contained in nickel fractions were collected and further run through a Heparin column #3 (AKTA FPLC). The chimeric ligase proteins were dialyzed into storage buffer. Specific examples of protein yield are shown in Table 1; and a polyacrylamide gel electrophoresis (SDS-PAGE) showing the purified protein post-dialysis is shown in FIG. 2.

### Example 3: RNAseH Fused Ligase Enzymes were Catalytically Inactive for RNaseH Activity

This example shows that the chimeric ligase enzymes with modified RNAse H domain fusions described herein were catalytically inactive for RNAse H activity. To demonstrate that the chimeric ligase enzymes were catalytically inactive for RNaseH activity, the chimeric ligase enzymes were assessed using a CE RNAseH assay. Specifically, an RNA template was hybridized to DNA cutting oligo as shown in FIG. 3A. The complex was diluted in buffer and treated with either 0.5 U/uL of catalytically active E. coli RNaseH (FIG. 3B) or 1 uM chimeric ligase (LigAT_C-RNAseH-ecoli) (FIG. 3C) for 2 hours at 37° C. The catalytic activity was analyzed by CE in SnapShot mode. As shown in FIG. 3B, the E. coli RNaseH generated cut products that were smaller in size than the Full-length RNA. In contrast, a reaction comprising the catalytically inactive LigAT_C-RNAseH-Ecoli chimeric ligase only had a single product, the full-length RNA (FIG. 3C). Thus, demonstrating that RNAseH fused ligase enzymes were catalytically inactive for RNaseH activity.

TABLE 1
Purification of LigAT_c-RNAseH-Ecoli

| | | | LigAT_C- | | LigAT_C- | |
|---|---|---|---|---|---|---|
| | | | RNAseH-Ecoli | | RNAseH-Ecoli | |
| Name | LigAT-HU | | (run #1, PE111) | | (run #2, PE130) | |
| MW (with SUMO) | 56.37 | | 64.22 | | 64.22 | |
| MW (post cleavage) | 43.75 | | 51.81 | | 51.81 | |
| Culture volume | 0.5 | L | 0.5 | L | 0.5 | L |
| yield (mg) | 5.62 | | 2.41 | | 2.5 | |
| Titer (mg/L) | 11.2 | mg/L | 4.82 | mg/L | 5.0 | mg/L |

### Example 4: RNAseH (Catalytically Inactive) Fused Ligase Enzymes were Active for RNA Templated DNA Ligation

This example shows that the chimeric ligases described herein had RNA templated DNA ligation activity. To determine whether the chimeric ligases were active for RNA templated ligation, padlocks reactions were prepared as shown in FIG. 4A. Each padlock reaction included a mixture of 3 padlocks (3 DNA probes with 5′-phosphate modification), 20 nM each for 60 nM total. Each padlock was annealed to an RNA template (80 nM). A ligation buffer was then combined with RNAse inhibitor (1 U/uL), annealed oligo (60 nM).

A chimeric ligase enzyme was then added to the padlock reactions at 1 uM final concentration, unless otherwise indicated. The reactions were pipette mixed and incubated at 37° C. for 2 hrs. The reactions were stopped by incubating the reactions at 85° C. for 5 min to heat kill the chimeric ligase. The reactions were then loaded on CE and analyzed by SnapShot mode.

As shown in FIG. 4B and FIG. 4C, the LigAT_C-RNAseH-ecoli chimeric enzyme was active for RNA-templated DNA ligation. In fact, the intensity of the ligated circular was greatly enhanced (FIG. 4C, 24,000 mfi) when compared to the product generated by the control (FIG. 4B, 4,000 mfi). The ratio of app-DNA to ligated product in a ligation reaction can be used to determine a ligase efficiency. In the control reaction, the mfi of the ligated product was about 4000 when compared to 10, 0000 for the app-DNA. In contrast, in FIG. 4C, the mfi of the ligated product was about 24,000 when compared to 14,000 mfi for app-DNA. This example shows that the chimeric ligase enzymes disclosed herein showed improved ligation activity when compared to control ligases.

### Example 5: RNAseH (Catalytically Inactive) Fused Ligase Enzymes were Thermostable Up to at Least 55° C.

This example shows that the chimeric ligases described herein had increased thermostability when compared to LigAT-HU. Specifically, the ligation reactions were conducted using DNA probes with 5′-phosphate modification and RNA template with the indicated ligase at the indicated temperature. Reactions were analyzed by capillary electrophoresis; a fully reacted circular product (% ligation) and intermediate product adenylpyrophosphoryl-DNA (% app-DNA) were quantified. LigAT_C-RNAseH-ecoli and LigAT_C-RNAseH-Sto showed superior ligation efficiency when compared to LigAT-HU.

To determine whether the chimeric ligase enzymes described herein were thermophilic, the CE padlock ligation assay described in Example 4 was performed at 37° C., 43° C., 48° C., 52° C., and 55° C. using 3 different buffers, a control ligation buffer, Buffer 1A, Buffer 1B, and Buffer 2B under control conditions (e.g., 2 hours). The buffers compositions are described in Table 2. Unless described otherwise, “control conditions” means 2 hours at 37° C.

FIGS. 5A-E show bar graphs demonstrating that the chimeric ligase enzymes described herein (FIGS. 5B-E) showed improved thermostability when compared to a control (FIG. 5A). In particular, catalytically inactive RNAseH fused ligase enzymes (e.g., LigAT_C-RNAseH-Ecoli or LigAT_C-RNAseH-Sto) were thermostable because they remained effective at producing ligation products at a temperature of up to at least 55° C. when tested in control buffer (FIGS. 5B and 5D; Buffer 1A (FIG. 5C); and Buffer 2A (FIG. 5E).

TABLE 2
Buffer Compositions

| | | | | | |
|---|---|---|---|---|---|
| Components | Control | Buffer 1A | Buffer 1B | Buffer 2B | Buffer 3A |
| | Buffer | (1x | (pH 8.5, | (pH 8, 0.85x | (pH 8.5, 1x |
| | (pH 8) | control, | 0.85x | control, but | control, KCL |
| | | pH 8.5) | control) | KCL changed | changed from |
| | | | | from about | about |
| | | | | 0.2-0.3M to | 0.2-0.3M |
| | | | | less than | to about 0.4- |
| | | | | 0.05M) | 0.5M KCL) |

In Buffer 1A, 1B, and Buffer 2B, over 75% of the nucleic acids at the end of the assay were final ligated products compared to less than 10% 5′-adenylpyrophosphoryl capped DNA (“app-DNA”) substrate at 37° C., 43° C., 48° C., 52° C., and 55° C. (FIGS. 5C and E). In the control, about 75% of the nucleic acids at the end of the assay were final ligated products at 37° C., 43° C., or 48° C. At 52° C. and 55° C., the percentage of final ligated products in the reaction remained above 25-30%. In contrast, the control ligase (LigAT-HU), produced only about 25% ligated product at 37° C. in control buffer, and that percentage was reduced to less than 5% at 55° C.

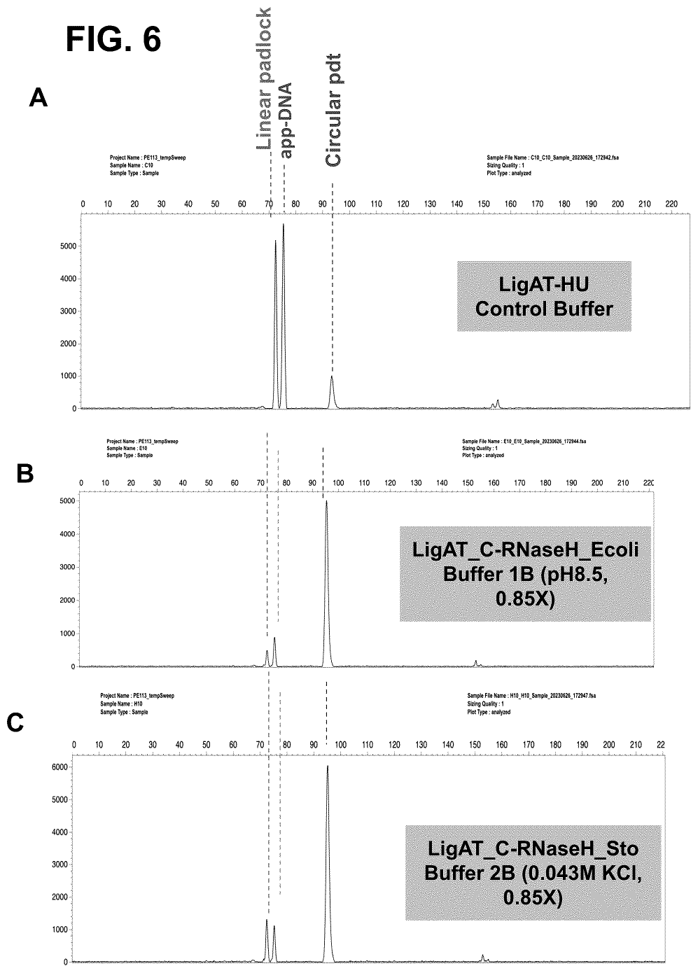

FIGS. 6A-C show exemplary electropherograms illustrating the results of the CE padlock ligation assay of FIGS. 5A-5E. FIG. 6A shows an electropherogram of a control ligation reaction (LigAT-HU) in a control Buffer. The majority of nucleic acids were either the substrates: linear padlock (mean fluorescence intensity (mfi)=5,000) or app-DNA (mfi-5,500). The mfi of the ligated product was less than 1,000. The. FIG. 6B shows an electropherogram of a ligation reaction comprising the chimeric ligase enzyme, LigAT_C-RNaseH_Ecoli in Buffer 1B. The mfis of the substrates were less than 1,000 for the linear padlock and app-DNA. The mfi of the ligated product was about 5,000. FIG. 6C shows an electropherogram of a ligation reaction comprising the chimeric ligase enzyme, LigAT_C-RNaseH_Sto in Buffer 2B. The mfis of the substrates were about 1,000 or less for the linear padlock and app-DNA. The mfi of the ligated product was about 6,000.

### Example 6: RNAseH (Catalytically Inactive) Fused Ligase Enzymes were Active In Situ and Enabled Higher Temperature Ligation Reactions

To demonstrates that the chimeric ligases were effective in high throughput in situ next-generation sequencing (NGS), the chimeric ligase enzymes were assessed using SNAIL probes for in situ sequencing. See e.g., PCT/US2021/018826.

A tissue sample was placed on a thin slide, and fixed using various techniques known in the art. The nucleic acid molecules (e.g., RNAs) in the sample were locked in place by embedding the sample in a hydrogel and crosslinking the nucleic acid molecules to the hydrogel. The tissue sample was first permeabilized with pepsin and HCl, and subsequently crosslinked.

Upon tissue crosslinking, mRNAs were targeted by probes and analyzed in situ, following highly multiplexed in situ imaging approaches (+e.g., probe hybridization), ligation using the chimeric enzyme disclosed herein, rolling circle amplification, followed by sequencing by ligation or sequencing by hybridization, with microscopy readouts.

FIGS. 7A-D shows fluorescent microscopy images of a whole tissue from in situ spatial assay performed using a microscope and analysis of optical signals for RNA sequence determination. Specifically, the chimeric ligase enzymes (e.g., catalytically inactive RNAseH fused ligase enzymes) described herein (FIGS. 7C-D) were active in situ and effective with higher temperature ligation when compared to control (FIGS. 7A-B).

FIGS. 8A-D show the high sensitivity and specificity of the chimeric ligase enzymes disclosed herein in in situ spatial assays using different conditions (buffers, and temperatures). Experiments were performed using formalin-fixed murine brain (FFmbrain) tissues, with padlock set PA_373, and cocktail priming. These data show that the sensitivity and specificity of the novel chimeric ligase enzymes described herein were greater or equal to control conditions. The control conditions used a control ligase enzyme (LigAT-HU) in a control buffer and the ligation reaction was performed for 2 hours at 37° C.

The sensitivity and specificity of the chimeric ligase enzymes were determined using the calculation and definition of gene tissue density with Q-score ≥20. FIG. 8A shows a schematic illustrating the calculation and definition of gene tissue density with Q-score ≥20 (in object per m³). This analysis is also called “Gene tissue density 3D.” Gene tissue density with Q-score ≥20 was defined as the number of genes divided by (tissue area times RCP thickness).

FIG. 8B shows the in situ spatial assay sensitivity analysis using 3D spatial data analysis. The sensitivity of the in situ assay was defined as gene tissue density with Q-score ≥20 in object (sample) per m³ (3D analysis) vs conditions (buffers and temperatures) shown on the x-axis. The conditions tested were two different buffers, Buffer 1A and Buffer 3A, compared to a control buffer and three different temperatures (37° C., 42° C., and 50° C.). All the percentages on the graph represent fold change from the control ligase, LigAT-HU at the control conditions. The control conditions of LigAT-HU ligation were 2 hours at 37° C. Tested enzymes included LigAT_C-RNAseH-Ecoli enzyme; LigAT_C-RNAseH-sto enzyme; and LigAT_C-RNAseH-RT enzyme. In situ assays including a LigAT_C-RNAseH-Ecoli enzyme were at least 8% more sensitive (e.g., higher quality transcript counts) than a control enzyme when used at 50° C. in Buffer 1A. In situ assays including a LigAT_C-RNAseH-sto enzyme were at least 9% more sensitive than a control enzyme when used at 50° C. in Buffer 1A. In situ assays including a LigAT_C-RNAseH-sto enzyme were at least 14% more sensitive than a control enzyme when used at 37° C. in Buffer 1A. In situ assays including a LigAT_C-RNAseH-RT enzyme were at least 26% more sensitive than a control enzyme when used at 37° C. in Buffer 1A.

FIG. 8C shows the sensitivity analysis using 2D spatial data analysis. The 2D means that the sensitivity of the in situ assay was defined as gene tissue density with Q-score ≥20 in object (sample) per m². In situ assays including a LigAT_C-RNAseH-Ecoli enzyme were at least 8% more sensitive than control conditions when used at 50° C. in Buffer 1A. In situ assays including a LigAT_C-RNAseH-sto enzyme were at least 12% more sensitive than control conditions when used at 37° C. in Buffer 1A. In situ assays including a LigAT_C-RNAseH-RT enzyme were at least 10% more sensitive than control conditions when used at 37° C. in Buffer 1A.

FIG. 8D shows the high specificity of the chimeric ligase enzymes disclosed herein in in situ spatial assays using an adjusted negative control probe rate. The adjusted negative control probe rate measured both erroneous decoding and off-target binding, relative to the total number of high-quality transcripts detected. An adjusted negative control probe rate represents an alternative way of showing the sensitivity of the in situ assay. The adjusted negative control probe count is obtained by multiplying: (total high quality decoded transcripts)*(adjusted negative control probe rate)*(the number of controls in the panel/the number of target genes in the panel). High quality transcripts are considered to be those with Q20 or above.

This example shows that the chimeric ligase enzymes were active in situ and that the chimeric ligase enzymes enabled higher temperature ligation.

The assay disclosed herein were conducted by altering the ligation enzyme and assay conditions as shown in the figures. Furthermore, the biological samples were freshly frozen mouse brain. The padlock set targeted 23-genes and contained negative control probes. Sensitivity metrics (FIG. 8B, FIG. 8C) and the specificity metrics (FIG. 8D) were compared for the workflows as a function of the ligation condition. The star shows that the same sensitivity and specificity as LigAT-HU, a control ligase, were obtained. These data show that LigAT_C-RNAseH-Ecoli enzyme showed the same sensitivity and specificity when incorporated in the workflow as the control ligase.

### Example 7: Chimeric Ligase Enzymes Showed Decreased Ligation Junction Bias

DNA ligases canonically join a 3′-hydroxyl of a nucleic acid to a 5′-phosphoryl group of another nucleic acid, for example, when both are hybridized to a complementary strand. To evaluate the novel chimeric ligases for ligation junction bias and fidelity, a capillary electrophoresis (CE) ligation assay was performed.

FIGS. 9A-C show an assay for monitoring ligation junction bias in vitro using orthogonal capillary electrophoresis (CE) assays. FIG. 9A shows schematics of 8 DNA probes and a template RNA for hybridization and ligation using a CE assay for monitoring ligation junction bias. The 8 probes are divided into: 4 different upstream oligonucleotides, each comprising a different nucleotide (e.g., A, C, G, and T) at the 3′end; and 4 different fluorophore-labeled downstream oligonucleotides, each comprising a different nucleotide (pA, pC, pG, and pT) at the 5′end. Each reaction well contained 4 different fluorophore-labeled downstream oligonucleotides, 4 different unlabeled upstream oligos, and one template RNA oligonucleotide having one of sixteen different dinucleotide sequences complementary to a ligation junction for a pair of probes. The length of each upstream oligonucleotide paired with one fluorophore-labeled downstream oligonucleotide was designed so that each of 16 reaction pairs (A_pA, A_pC, A_pG, A_pT, C_pA, C_pC, C_pG, C_pT, G_pA, G_pC, G_pG, G_pT, T_pA, T_pC, T_pG, T_pT) yields a ligation product of a different length, which can be differentiated by electropherogram. The probe pairs were hybridized to a template RNA and the pair was ligated together by a chimeric ligase enzyme (a ligase fused to an RNAseH domain) disclosed herein or a control enzyme (a ligase fused to a DNA-binding domain) to generate a ligated product. The template RNA was digested and the ligated DNA product was assayed by capillary electrophoresis.

FIG. 9B shows an exemplary electropherogram from a CE assay demonstrating the separation of the substrates (probe pairs) and the 16 expected exemplary ligated products described in FIG. 9A. All 16 products were separable in the CE assay.

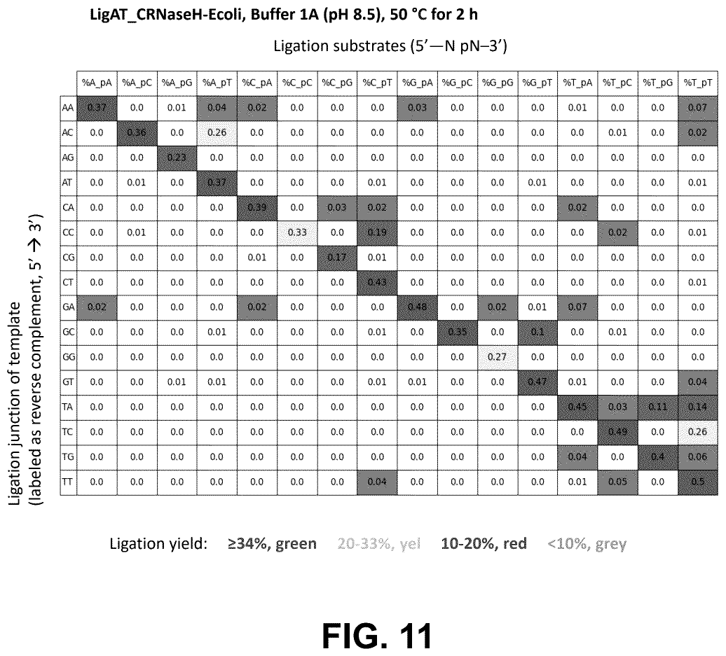

FIG. 9C shows a schematic illustrating the interpretation of FIG. 10A and FIG. 11. Specifically, for ligation junction “AC,” the ligation substrate comprised an upstream oligonucleotide comprising an A at the 3′end; and a fluorophore-labeled downstream oligonucleotide comprising pC to form a reaction pair A_pC from 5′→3′ which formed a Watson-Crick bond with a complementary TG (3′→5′) on the template RNA. The two bases at the ligation junction on the template RNA are “AC” (reverse complement of the 5′→3).

FIGS. 10A-B show results of a multiplexed ligation fidelity and bias CE profiling assay using a control ligase enzyme (LigAT-HU) demonstrating that the control ligase enzyme showed strong ligation junction bias. FIG. 10A shows an exemplary analysis table of a multiplexed ligation efficiency and fidelity and bias CE profiling assay using a control ligase enzyme (LigAT-HU). The top row shows the 16 as described in FIG. 9A (ligation substrate/product read from 5′→3′ (5′-N pN-3′)). The left column shows the two bases at the ligation junction of the RNA template. The bases are labeled as a reverse complement from 5′→3′ (AA, AC, AG, AT, CA, CC, CG, CT, GA, GC, GG, GT, TA, TC, TG, TT). The Watson-Crick pairings of a template dinucleotide motif with the substrate pairs are shown on the diagonal, which represents a fully matched probe set (as shown in FIG. 9C above for the AC ligation reaction). Data outside the diagonal represent ligations that occurred while substrates were hybridized to a mismatched template.

FIG. 10B shows a list ranking the ligation yield/efficiency of the control ligase enzyme, for substrates hybridized to a template with correct base-pairing. As can be seen in FIG. 10B, the reaction yield was less than 10% for five of the sixteen substrate combinations properly base-paired with the substrate: AG, CC, CG, GC, and GG ligation junctions.

FIG. 11 shows results of multiplexed ligation fidelity and bias profiling assays using a chimeric ligase enzyme described herein (LigAT_C-RNaseH-Ecoli). As can be seen in FIG. 11, ligase efficiency was increased for the LigAT_RNaseH chimeric enzyme, with all sixteen substrates generating at least a 10% yield of the correct product, as can be seen by the absence of gray boxes along the diagonal of the table. In contrast, in the reactions using the control ligase, five of the sixteen substrates generated lower than a 10% yield of the correct product, as can be seen by the five gray boxes along the diagonal in FIG. 10A.

Thus, FIG. 11 demonstrates that an exemplary chimeric ligase enzyme described herein showed decreased ligation junction bias when compared to a control. For correctly matched probe sets, the reaction yield was AA (37%), AC (36%), AG (23%), AT (37%), CA (39%), CC (33%), CG (17%), CT (43%), GA (48%), GC (35%), GG (27%), GT (47%), TA (45%), TC (49%), TG (40%) and TT (50%). Surprisingly, and unlike the control ligase, none of the ligations yields for properly base-paired substrates were under 10%. In fact, all sixteen generated a yield of 17% or higher.

The template for row ‘AA’ has sequence (ligation junction is bolded): rGrArGrCrArGrUrUrArArGrCrCrArGrArGrCrCrArArCrUrUrArCrUrCrCrUrCrUrGrUrGr GrArArGrUrCrUrCrUrCrC (SEQ ID NO: 41). The % A_pA refers to ligation of the following substrates: substrate (A): GAG ACT TCC ACA GAG GAG TA (SEQ ID NO: 42); and Substrate (pA):/5Phos/AGT TGG CTC TGG CTT AAC TGC GCT AAC TTC-fluorophore (SEQ ID NO: 43).

The data demonstrate that the novel chimeric ligase enzymes disclosed herein increase ligation efficiency, when compared to a control ligase that is not fused to a catalytically inactive RNAseH domain (e.g., is instead fused to a DNA binding domain).

### Example 8: Chimeric Ligase Enzymes Showed Less Ligation Junction Bias in a Ligation-Based Sequencing Assay

This example shows that chimeric ligase enzymes described herein showed less ligation junction bias when compared to LigAT-HU in in situ a ligation-based sequencing assay, as described above. The relative sensitivity of padlock probes as a function of their ligation junction was compared for data obtained from workflows employing either LigAT_C-RNaseH-Ecoli or a control enzyme (LigAT-HU). The ligation-based sequencing assay was an in situ assay, performed using formalin-fixed and paraffin embedded (FFPE) human cancer samples. The padlock set targeted 1600-genes and contained negative control probes.

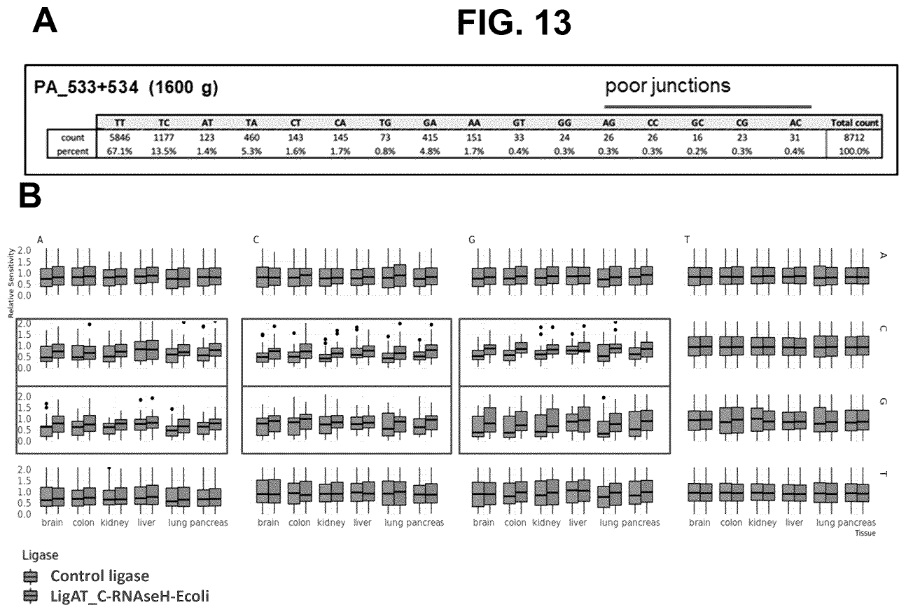

FIGS. 13A-B show results of in situ spatial assays using an exemplary chimeric ligase enzyme described herein (e.g., LigAT_C-RNaseH-Ecoli enzyme) and a control. In particular, these results illustrate that the chimeric ligase enzyme improved the poor ligation junction sensitivity that was observed with a ligation reaction comprising a control enzyme (e.g., LigAT-HU) while maintaining other junctions.

FIG. 13A shows a table summarizing the UMI counts and percent yield for each nucleotide pair at the ligation junction and demonstrating that the control enzyme showed poor junction sensitivity at ligation junctions GG, AG, CC, GC, CG, and AC in the in situ assay.

FIG. 13B shows graphs illustrating the relative sensitivity of the exemplary chimeric ligase enzyme (e.g., LigAT_C-RNaseH-Ecoli enzyme) compared to the control enzyme (e.g., LigAT-HU) in an in situ assay. Boxes identified data from padlocks with LigAT-HU showing the ligation poor junctions ‘AC’, ‘AG’, ‘CC’, ‘CG’, ‘GC’, and ‘GG’. The relative sensitivity of the exemplary chimeric ligase enzyme was significantly improved at the AC, AG, CG, GC, and GG junctions (shown in boxes). In particular, the relative sensitivity of the exemplary chimeric ligase enzyme was significantly improved at the CC junction in all tumor tissues tested (brain, colon, kidney, liver, lung, and pancreas). Non-highlighted ligation junction datasets showed similar sensitivity when either LigAT_C-RNaseH-Ecoli or LigAT-HU was used.

### Example 9: Chimeric Ligase Enzymes Decreased Ligation Junction Bias of Uromodulin (UMOD) or Aquaporin 2 (AQP2) Probes

Furthermore, the decreased ligation junction bias of LigAT_C-RNaseH-Ecoli when compared to LigAT-HU was observed at the individual padlock probe level (e.g., uromodulin gene (UMOD) or Aquaporin 2 (AQP2) gene). The sample tested was a FFPE human kidney. The padlock set targeted 1000-genes and contained negative control probes.

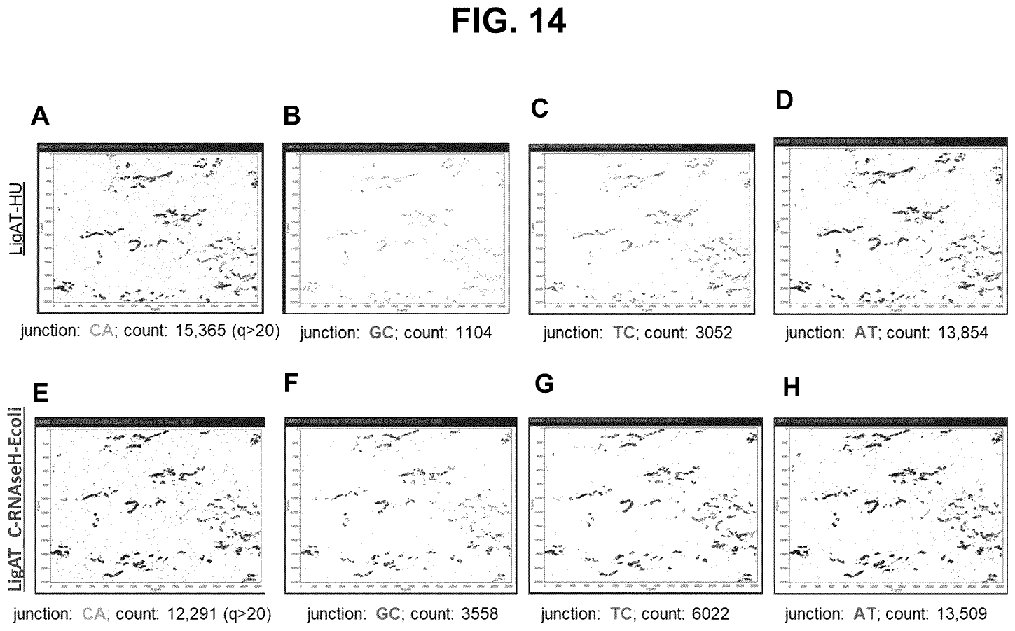

FIGS. 14A-H show Uromodulin (UMOD) gene density data with Q-score ≥20 from in situ spatial assays using an exemplary chimeric ligase enzyme described herein (e.g., LigAT_C-RNaseH-Ecoli enzyme) and 4 ligation junction probes (CA, GC, TC, and AT). The chimeric ligase enzyme (FIGS. 14E-H) showed less ligation junction bias in an in-situ spatial assay from a FFPE human Kidney sample when compared to a control enzyme (FIGS. 14A-D). The sensitivity of the in situ assay was defined as gene tissue density with Q-score ≥20 in object (sample). The ligation yields were 20-33% (CA), 10-20% (GC), and ≥34% (TC and AT). However, at ligation junction “GC,” the UMI count in the control reaction was 1104, while the count in the LigAT_C-RNaseH-Ecoli enzyme was about 3558. Thus, the sensitivity of the UMOD probe was significantly improved at the ligation junctions ‘GC’ using the LigAT_C-RNaseH-Ecoli fusion ligase, as compared to when the control enzyme, LigAT-HU, was used.

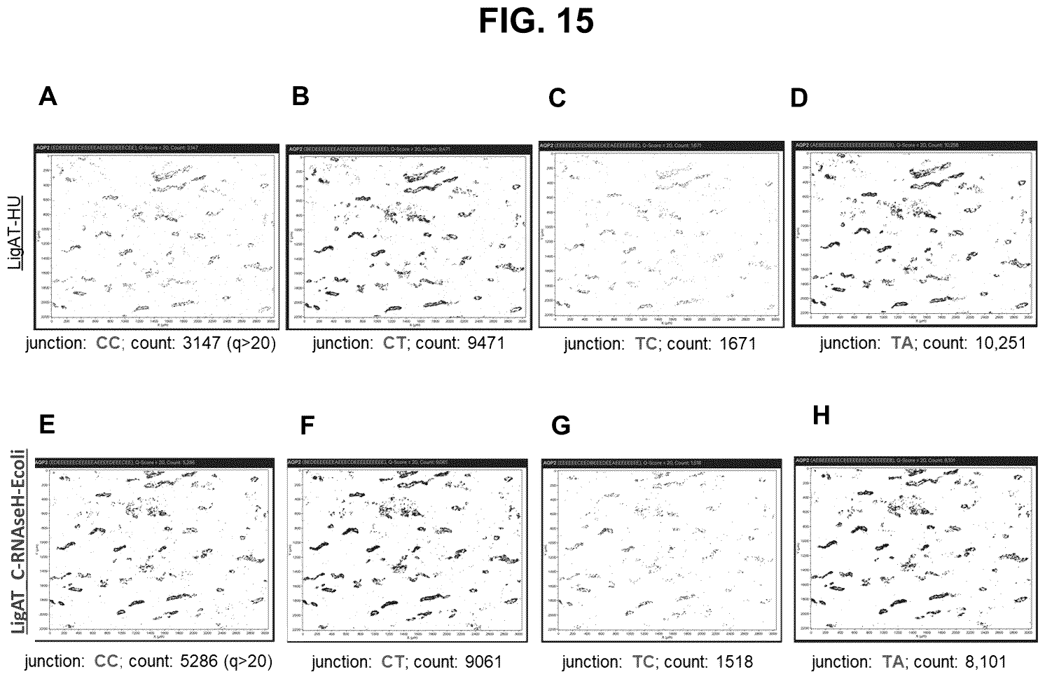

Conversely, FIGS. 15A-H show Aquaporin 2 (AQP2) gene density data with Q-score ≥20 from in situ spatial assays using an exemplary chimeric ligase enzyme described herein (e.g., LigAT_C-RNaseH-Ecoli enzyme) and 4 ligation junction probes (CC, CT, TC, and TA). An exemplary chimeric ligase enzyme (FIGS. 15E-H) showed less ligation junction bias in the in situ spatial assay from a FFPE human Kidney sample when compared to the control enzyme (FIGS. 15A-D). The ligation yields were 10-20% (CC), and ≥34% (CT, TC, and TA). However, at ligation junction “CC,” the UMI count in the control reaction was 3147, while the count in the LigAT_C-RNaseH-Ecoli enzyme was about 5286. Thus, the sensitivity of the AQP2 probe was significantly improved at the ligation junctions ‘CC’ in the modified Xenium workflow when LigAT_C-RNaseH-Ecoli was used compared to when LigAT-HU was used.

The probes for the ‘UMOD’ and ‘AQP2’ genes, which were poorly ligated at ligation junctions ‘GC’ and ‘CC’ when LigAT-HU was used, showed improved sensitivity when an engineered chimeric ligase enzyme described herein (e.g., LigAT_C-RNaseH-Ecoli) was used.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

1. A chimeric ligase polypeptide comprising:
(a) a ligase polypeptide domain; and
(b) a catalytically inactive ribonuclease H (RNAseH) polypeptide domain.

2. The chimeric ligase polypeptide of claim 1, wherein the catalytically inactive RNAseH polypeptide domain is located at the N-terminus of the ligase polypeptide domain.

3. The chimeric ligase polypeptide of claim 1, wherein the catalytically inactive RNAseH polypeptide domain is located at the C-terminus of the ligase polypeptide domain.

4. The chimeric ligase polypeptide of claim 1, wherein the ligase polypeptide domain and the catalytically inactive RNAseH polypeptide domain are operably linked by a linker.

5. The chimeric ligase polypeptide of claim 1, wherein the ligase polypeptide domain comprises a T3 DNA ligase, a T4 DNA ligase, a T7 DNA ligase, a HiFi Taq DNA ligase, a 9° N™ DNA Ligase, an E. coli DNA ligase, a Chlorella virus DNA ligase, a Paramecium bursaria Chlorella virus ligase, a Syngen Nebraska virus 5 ligase, or an Acanthocyslis turfacea chlorella virus ligase.

6. The chimeric ligase polypeptide of claim 1, wherein the ligase polypeptide domain comprises a Acanthocyslis turfacea chlorella virus ligase selected from the group consisting of ATCVMO0605SPH_239L, ATCV1_Z187L, ATCVWI0606_243L, ATCVNEJV2_239L, ATCVMN08101_251L, ATCVTN60342_234L, ATCVGM07011_240L, and ATCV-1 (LigAT).

7. The chimeric ligase polypeptide of claim 1, wherein the ligase polypeptide domain comprises a Paramecium bursaria Chlorella virus ligase selected from the group consisting of PBCV-1, KS1B, AN69C, CvsA1, NE-JV-4, CviKI, MA1D, NY2B, NYs1, PBCV-AR158, IL-5-2s1, MA1E, PBCV-IL3A, PBCV-NY2A, and AR158_C658R.

8. The chimeric ligase polypeptide of claim 6, wherein the ligase polypeptide domain comprises ATCV-1.

9. The chimeric ligase polypeptide of claim 1, wherein the ligase polypeptide domain comprises a T4 ligase.

10. The chimeric ligase polypeptide of claim 1, wherein the catalytically inactive RNAseH polypeptide domain is selected from the group consisting of Pyrococcus furiosus RNAseH, Pyrococcus horikoshi RNAseH, Pyrococcus woosii RNAseH, Bacillus sterothermophilus RNAse, Bacillus caldophilus RNAse, Thermococcus litoralis RNAseH II, Thermotoga neapolitana RNAse, Sulfolobus acidocaldarius RNAse, Thermoplasma acidophilum RNAseH, Thermus thermophilus RNAseH, Thermus thermophilus RNAseHI, Thermus thermophilus RNAseHII, Thermus thermophilus RNAseHIII, Sulfobolus tokodaii RNAseH, Escherichia coli RNAseH, and a viral reverse transcriptase RNAse domain.

11. The chimeric ligase polypeptide of claim 1, wherein the catalytically inactive RNAseH polypeptide domain comprises a catalytically inactive E. coli RNAseH polypeptide.

12. The chimeric ligase polypeptide of claim 1, wherein the catalytically inactive RNAseH polypeptide domain comprises a catalytically inactive Sulfobolus tokodaii RNAseH polypeptide.

13. The chimeric ligase polypeptide of claim 1, wherein the catalytically inactive RNAseH polypeptide domain comprises a catalytically inactive viral reverse transcriptase RNase domain.

14.-15. (canceled)

16. The chimeric ligase polypeptide of claim 4, wherein the linker comprises an amino acid sequence having at least about 90%, at least about 91%, at least about 92%, at least about 93%, at least about 94%, at least about 95%, at least about 96%, at least about 97%, at least about 98%, or at least about 99% sequence identity to any one of SEQ ID NO: 17, 18, 19, or 20.

17. The chimeric ligase polypeptide of claim 1, wherein the catalytically inactive RNAseH polypeptide domain comprises an amino acid sequence having at least about 90%, at least about 91%, at least about 92%, at least about 93%, at least about 94%, at least about 95%, at least about 96%, at least about 97%, at least about 98%, or at least about 99% sequence identity to any one of SEQ ID NO: 13, 31, or 33.

18. The chimeric ligase polypeptide of claim 1, wherein the catalytically inactive RNAseH polypeptide domain comprises an amino acid sequence set forth in SEQ ID NO: 13, 31, or 33.

19. The chimeric ligase polypeptide of claim 1, wherein the catalytically inactive RNAseH polypeptide domain comprises SEQ ID NO: 31.

20. The chimeric ligase polypeptide of claim 1, wherein the catalytically inactive RNAseH polypeptide domain comprises SEQ ID NO: 33.

21. The chimeric ligase polypeptide of claim 1, wherein the catalytically inactive RNAseH polypeptide domain comprises SEQ ID NO: 13.

22. (canceled)

23. The chimeric polypeptide of claim 1, wherein the ligase polypeptide domain comprises an amino acid sequence having at least 90%, at least 91%, at least 92%, at least 93%, at least 94%, at least 95%, at least 96%, at least 97%, at least 98%, or at least 99% identity to SEQ ID NO: 29, 36, 38, or 39.

24.-28. (canceled)

29. The chimeric ligase polypeptide of claim 1, wherein the chimeric ligase comprises an amino acid sequence that is at least 90%, at least 92%, at least 95%, at least 97%, at least 98%, or at least 99% identical to SEQ ID NO: 2, 4, 6, 8, 10, and 12.

30.-49. (canceled)

50. A method for ligating double-stranded DNA or single-stranded DNA, the method comprising contacting under suitable conditions a sample comprising a nucleic acid template and DNA probes with a composition comprising a chimeric ligase polypeptide of claim 1.

51. The method of claim 50, wherein the composition comprises a ligation buffer, and wherein the ligation buffer has a pH:
(a) in the range of about 8 to about 8.5; or
(b) of at least about 8.0, 8.1, 8.2, 8.3, 8.4, or 8.5.

52. The method of claim 50, wherein the sample comprises a cell, a cell bead, a permeabilized cell, a nucleus, a tissue, or a cellular preparation.

53. The method of claim 52, wherein the nucleus is permeabilized.

54. The method of claim 50, wherein the sample comprises cells in suspension, fresh cells, fixed cells, or cells and tissues immobilized on various solid surfaces.

55. The method of claim 50, wherein the nucleic acid template is an RNA, a DNA, or a nucleic acid comprising an unnatural nucleotide.

56.-75. (canceled)
